(19) United States
(12) Patent Application Publication
(10) Pub. No.: US 2025/0263473 A1
(43) Pub. Date: Aug. 21, 2025

(54) BISPECIFIC BINDING AGENT

(71) Applicant: THE FRANCIS CRICK INSTITUTE LIMITED, London, Greater London (GB)

(72) Inventors: Caetano REIS E SOUSA, London (GB); Tomas CASTRO-DOPICO, London (GB); Raj MEHTA, London (GB); Brian Edward VASH, Cambridge, MA (US); Stephanie J. MAIOCCO, Cambridge, MA (US)

(21) Appl. No.: 18/858,525

(22) PCT Filed: Apr. 21, 2023
(86) PCT No.: PCT/EP23/60439

§ 371 Date: Oct. 21, 2024

(30) Foreign Application Priority Data
Apr. 21, 2022 (GB) 2205808.5
Oct. 7, 2022 (GB) 2214753.2

(51) Int. Cl. C07K 16/18; A61K 39/00; A61K 45/06; A61P 35/00; C07K 16/28; G01N 33/574

(52) U.S. Cl. CPC C07K 16/18; A61K 45/06; A61P 35/00; C07K 16/283; G01N 33/57484; A61K 2039/505; A61K 2039/54; C07K 2317/14; C07K 2317/31; C07K 2317/52; C07K 2317/75; G01N 2333/70539

(57) ABSTRACT

The present invention relates to bispecific binding agents comprising an F-actin binding moiety and an Fc domain for use in medicine, which allows for simultaneous binding of an Fc receptor and F-actin. This allows necrotic cell antigens to be presented to the immune system. The invention also provides bispecific binding agents and methods of using the agent to identify a tumour antigen.

### FIELD OF THE INVENTION

The present invention relates to agents that interact with the presentation of necrotic cell antigens. The invention provides bispecific binding agents that allow the necrotic cell antigens to be presented to the immune system. The invention further provides nucleic acids and plasmids encoding the construct as well as medical uses thereof.

### BACKGROUND

Type 1 conventional dendritic cells (cDC1) are indispensable for effective anti-tumour immunity (Wculek, 2020). This is, in part, attributable to their ability to acquire antigens from tumour cells, migrate to draining lymph nodes, and prime cancer-specific CD8⁺ T cells (Alloatti, 2017; Salmon, 2016; Theisen 2018). This requires the presentation of those antigens on MHC class I molecules, a process termed cross-presentation (XP). Although many antigen presenting cells (APC), such as type 2 cDC (cDC2), may be capable of performing XP per se, cDC1 appear specialised for this process in the context of cell-associated antigens. Notably, cDC1 represent only a minority of APCs within tumours, with cDC2 and macrophages being the major APCs in tumours.

Within tumours, a possible source of antigens for XP is necrotic cell debris, which is avidly internalised by cDC1 (Galluzzi, 2017). cDC1 express high levels of the C-type lectin receptor DNGR-1 (also known as CLEC9A), which recognises filamentous actin (F-actin) exposed on necrotic cells (WO 2013/088136; Hanč, 2015; Zhang, 2012; Ahrens 2012). Notably, DNGR-1 expression is highly restricted to cDC1 in both mice and humans (Poulin, 2012) and acts as a receptor dedicated to XP of necrotic cell-associated antigens. Upon binding to F-actin via its C-type lectin domain (CTLD), DNGR-1 triggers SYK signalling, which causes rupture of ligand-containing phagosomes, release of antigenic material into the cDC1 cytosol, and its entry into the endogenous MHC class I presentation pathway (Sancho, 2009; Canton, 2021; WO 2009/013484A1). Importantly, as F-actin exposure is associated with pathological cell death (e.g., necrosis) rather than apoptosis, DNGR-1 may act as a necrotic cell sensor to specifically couple recognition of tissue damage to the activation of a cytotoxic CD8⁺ T cell response. As such, DNGR-1 can play an important role in the priming of cytotoxic CD8⁺ T cells against cytopathic viruses or tumours. WO 2022/163809 provides molecules that bind to a component which is exposed upon necrotic cell death.

The activity of DNGR-1 is regulated by secreted gelsolin (sGSN), one of two abundant actin-binding proteins (the other being Gc globulin) that contribute to the removal of potentially pathological actin filaments released from or exposed by dying cells following tissue damage (Giampazolias, 2021). sGSN binds to F-actin in a Ca²⁺-dependent manner and severs the filaments for subsequent depolymerisation, which is facilitated by Ca²⁺-independent sequestering of monomeric G-actin by Gc globulin. In this way, sGSN can mask the activity of DNGR-1 by preventing binding to F-actin.

DNGR-1 itself is a type II transmembrane protein, with the extracellular C-terminal CTLD of DNGR-1 connected via a neck region to a transmembrane (TM) domain and subsequent hemlTAM (hemi-immunoreceptor tyrosine-based activation motif)-containing N-terminal intracellular tail (Huysamen, 2008; Sancho, 2008). The hemITAM motif is required for Syk binding. Mice are known to have five different isoforms of DNGR-1, of which only two isoforms possess the entire ligand-binding domain and TM region. These isoforms are termed “long” (isoform 4) and “short” (isoform 1). Humans only have a single isoform, which corresponds to the “short” mouse isoform. The “long” mouse isoform is distinguished from the “short” mouse isoform and the human isoform by an extra exon which codes for an additional 26 amino acids in the neck region.

Fc receptors are expressed on various immune cells and are grouped according to which type of antibody they bind. For example, Fcγ receptors (FcγR) bind to lgG. FcγRs comprise many activating-type receptors and a unique inhibitory receptor, FcγRIIB. Binding of the Fc domain of an antibody to its activatory receptor leads to activation of the cell expressing the Fc receptor. Activatory Fcγ receptors are widely expressed ITAM-bearing Syk-coupled cell surface glycoproteins that bind to the Fc domain of IgG antibodies (Giulliams, 2014). Upon binding to IgG-opsonised targets, they can induce potent cellular immune responses in infection, autoimmunity, and cancer. Importantly, FcγR exhibit overlapping characteristics with DNGR-1, as well as unique ones (Table 1), and have been shown to promote Syk-dependent XP of IgG-bound antigens through an ill-defined pathway (Regnault, 1999). Furthermore, tumour-binding antibodies have been shown to drive anti-cancer immunity through FcγR-mediated cDC activation (Carmi, 2015; Carmi 2019).

TABLE 1
Shared and unique features of DNGR-1 and FcγR

| Shared features | Unique features of DNGR-1 | Unique features of FcγR |
|---|---|---|
| ITAM/Syk-dependent | cDC1-restricted expression | Broader expression profile (cDC1, |
| signalling | Does not induce cellular activation | cDC2, monocyte-derived cells, |
| Cross-presentation of | Does not induce antigen | macrophages, granulocytes, NK |
| exogenous antigen on | presentation on MHC-II for CD4⁺ T | cells, B cells) |
| MHC-I via the cytosolic | cells | Antigen presentation of IgG |
| pathway in cDC | | immune complexed antigen on |
| | | MHC-II for CD4⁺ T cells |
| | | Immune cell activation and |
| | | migration |

The present invention has been devised in light of the above considerations.

### SUMMARY OF THE INVENTION

The inventors sought to utilise FcR to maximise the number and type of APCs capable of sensing necrotic cell debris by combining an F-actin binding moiety with an FcR-binding moiety. This allows F-actin exposure on necrotic cells to be coupled to the activation of an immune response. Furthermore, the invention enhances delivery of necrotic cell debris, including necrotic tumour cell debris, to diverse APC populations to ensure that those APCs 1) acquire greater quantities of necrotic cell-associated antigens for MHC class II presentation (in addition to XP); and 2) are exposed to damage-associated molecular patterns (DAMPs) from the necrotic cells that contribute to APC activation. The necrotic cell debris may be within the tumour microenvironment (TME), and the antigen may be a necrotic tumour cell-associated antigen. Therefore, the FcR signalling leads to enhanced anti-cancer immunity. Furthermore, the invention promotes FcR signalling to lead to additional Syk-dependent APC activation.

In one example, the F-actin binding moiety is the C-type lectin domain (CTLD) of DNGR-1. In this way, necrotic cell sensing and XP following F-actin exposure is not restricted to rare DNGR-1-expressing cDC1 (which represent only a minority of APCs within tumours) but can be performed by more abundant APCs through the FcγR signalling pathway, leading to enhanced CD8⁺ T cell priming and immunity.

Fusion proteins of DNGR-1 and FcγR-binding moieties are known, such as those supplied by R&D Systems for research purposes. Those fusion proteins find applications in ligand identification, ligand binding and visualisation studies (Iborra, 2012; Uto, 2016). However, they have not been used for antigen delivery, XP and cell activation purposes. The inventors have shown that the human constructs supplied by R&D Systems are unable to functionally engage Fc receptors and therefore cannot trigger XP of necrotic cell antigens to T cells (Example 10 and FIG. 11), or other cellular processes downstream of the Fc receptor. Mayer et al. (2018) also discloses fusion proteins of DNGR-1 and an Fc domain. However, these fusion proteins exclusively use a wild-type Fc domain, and are used only as tools to screen for interactions between bacteria and C-type lectin receptors (CLRs).

In a first aspect, the invention provides a bispecific binding agent comprising an F-actin binding moiety and an Fc receptor-binding moiety for use in medicine, wherein the bispecific binding agent is capable of simultaneously (i) binding F-actin via the F-actin binding moiety; and (ii) binding an Fc receptor via the Fc receptor-binding moiety. Preferably, the Fc receptor-binding moiety is an Fc domain, and the Fc domain comprises an amino acid sequence that has been mutated from that of a wild type Fc domain such that an Fc receptor binds to the mutated Fc domain with higher affinity and/or avidity than the affinity and/or avidity of the Fc receptor for the wild type Fc domain. This simultaneous binding enables the bispecific binding agent to couple necrotic cell sensing to various downstream cellular responses. This response may include XP, MHC class II presentation, MHC Class II expression, presentation of exogenous antigens by MHC Class II, XP of MHC Class I antigens, phagocytosis of macromolecular complexes comprising an antigen, activation of Fc receptor-expressing cells, induction of type I interferon (IFN) expression, and/or delivery of macromolecular complexes comprising an antigen to the cytosol of Fc receptor-expressing cells. In some embodiments, the F-actin binding moiety is specific for F-actin. In some embodiments, the F-actin binding moiety has greater specificity for F-actin relative to G-actin. In some embodiments, the specificity for F-actin is between 2- and 100-fold greater than for G-actin. In some embodiments, the specificity for F-actin is between 2- and 10-fold greater than for G-actin. In some embodiments, the specificity for F-actin is at least 2-fold greater than for G-actin. In some embodiments, the specificity for F-actin is at least 5-fold greater than for G-actin. In some embodiments, the specificity for F-actin is at least 10-fold greater than for G-actin. In some embodiments, the specificity for F-actin is at least 100-fold greater than for G-actin. Specificity is explicitly intended to encompass affinity and/or avidity. In some embodiments, the F-actin binding moiety binds both F-actin and G-actin. For the avoidance of doubt, the invention does not encompass bispecific binding agents which bind to G-actin but do not bind to F-actin. The Fc receptor-binding moiety is specific for an Fc receptor.

In related aspects of the invention, there is provided a method of treatment comprising administering a bispecific binding agent as described herein to a subject in need thereof.

In other related aspects, the invention provides the use of a bispecific binding agent as described herein for the manufacture of a medicament for the treatment of cancer,

In some embodiments, the Fc receptor-binding moiety preferentially binds an activatory Fc receptor relative to an inhibitory Fc receptor. In some embodiments, the specificity for an activatory Fc receptor is between 2- and 100-fold greater than for an inhibitory Fc receptor. In some embodiments, the specificity for an activatory Fc receptor is between 2- and 10-fold greater than for an inhibitory Fc receptor. In some embodiments, the specificity for an activatory Fc receptor is at least 2-fold greater than for an inhibitory Fc receptor. In some embodiments, the specificity for an activatory Fc receptor is at least 5-fold greater than for an inhibitory Fc receptor. In some embodiments, the specificity for an activatory Fc receptor is at least 10-fold greater than for an inhibitory Fc receptor. In some embodiments, the specificity for an activatory Fc receptor is at least 100-fold greater than for an inhibitory Fc receptor. Specificity is explicitly intended to encompass affinity and/or avidity.

In some embodiments, the F-actin binding moiety comprises the CTLD of DNGR-1. In some embodiments, the CTLD of DNGR-1 is the CTLD of human DNGR-1. The CTLD of DNGR-1 specifically binds polymeric F-actin but not monomeric G-actin. Therefore, DNGR-1 differentiates between polymeric actin displayed on necrotic cells and monomeric G-actin.

In some embodiments, the F-actin binding moiety is linked to the Fc receptor-binding moiety via a linker that allows the Fc receptor-binding moiety to bind an Fc receptor and allows the F-actin binding moiety to bind F-actin at the same time. In these embodiments, the linker enables simultaneous binding of the two components of the bispecific binding agent, by providing sufficient flexibility and/or distance between the two components to enable activation of various downstream cellular responses. DNGR-1 is a type II membrane protein, which means that its orientation is reversed relative to type I membrane proteins, with the C-terminal domain targeted to the ER lumen. Therefore, in some embodiments where the F-actin binding moiety is the CTLD of DNGR-1, the CTLD of DNGR-1 may be fused to the Fc receptor-binding moiety via the DNGR-1 C-terminus, not its N-terminus. A linker may be advantageous in these embodiments to provide sufficient flexibility and/or distance between the Fc receptor-binding moiety and the CTLD of DNGR-1. In other embodiments, the CTLD of DNGR-1 may be fused to the Fc receptor-binding moiety via the DNGR-1 N-terminus. In these embodiments, different linkers may be used relative to embodiments fused via the DNGR-1 C-terminus. The inventors have discovered that the known human DNGR-1-human Fc (hC9-hFc) construct of the prior art (R&D Systems) is unable to functionally engage Fcγ receptors. The human DNGR-1 and human Fc in this construct are linked by an IEGR linker. Without wishing to be bound by any particular theory, the inventors hypothesise the inability to functionally engage Fcγ receptors may be due to the short IEGR linker.

In some embodiments, the Fc receptor-binding moiety is engineered to preferentially bind an activatory Fc receptor relative to an inhibitory Fc receptor. In some embodiments, the Fc receptor-binding moiety is engineered to preferentially bind an activatory Fcγ receptor relative to an inhibitory Fcγ receptor.

In preferred embodiments, the Fc receptor-binding moiety comprises an Fc domain. Fc domains comprise two identical heavy chain fragments, each comprising two or three constant domains.

In some embodiments, the Fc domain comprises an Fc domain of an immunoglobulin that is capable of binding and triggering activatory Fc receptors. Human activatory Fc receptors include the Fc receptors FcγRI, FcγRIIA and FcγRIIIA. Murine activatory Fcγ receptors include FcγRI, FcγRIII and FcγRIV.

In some embodiments, the Fc domain is engineered to preferentially bind an activatory Fcγ receptor relative to an inhibitory Fcγ receptor.

In some embodiments, the Fc domain is a human Fc domain.

In some embodiments, the Fc domain is from the pro-inflammatory immunoglobulin human IgG1 or human IgG3. Human IgG1 Fc has higher affinity for activating receptors, particularly FcγRI and FcγRIIA (Castro-Dopico, 2019), as compared to inhibitory receptors.

In some embodiments, the Fc domain is from the pro-inflammatory immunoglobulin murine IgG2a. Murine IgG2a has higher affinity for the activating receptors FcγRI and FcγRIV (Castro-Dopico, 2019).

In some embodiments, the Fc domain comprises an amino acid sequence that has been mutated from that of a wild type Fc domain such that the mutated Fc domain binds to an activatory Fc receptor with higher affinity and/or avidity than the affinity and/or avidity of the wild type Fc domain for the activatory Fc receptor. In some embodiments, the mutant Fc domain is a GASDALIE mutant Fc domain, comprising mutations corresponding to G236A, S239D, A330L and I332E (EU numbering relative to WT human IgG1). This mutant has been shown to have an activating effect on hFcγRI, hFcγRIIA (R131 and H131), and hFcγRIIIA (V158 and F158) (Bruhns, 2015). In some embodiments, the mutant Fc domain is a GA mutant Fc domain, comprising a mutation corresponding to G236A. In some embodiments, the mutant Fc domain is an ALIE mutant Fc domain, comprising mutations corresponding to A330L and I332E. In some embodiments, the mutant Fc domain comprises a mutation selected from one or more of the mutations set out in Table 2 below, which is adapted from Table 1 of Saunders (2019), which is incorporated by reference herein in its entirety.

TABLE 2
Fc modifications to increase affinity and/or avidity
to Fc receptors. Modified from Saunders (2019).

| Mutation | Abbreviated name |
|---|---|
| Ser298Ala/Glu333Ala/Lys334Ala | AAA |
| Ser239Asp/Ala330Leu/Ile332Glu | DLE |
| Ser239Asp/Ile332Glu | DE |
| Gly236Ala/Ser239Asp/Ala330Leu/Ile332Glu | GASDALIE |
| Gly236Ala | GA |
| Ser239Asp/Ile332Glu/Gly236Ala | DAE |
| Leu234Tyr/Gly236Trp/Ser298Ala | YWA |
| Phe243Leu, Arg292Pro, Tyr300Leu, | Variant 18 |
| Val305Ile, and Pro396Leu | |
| Lys326Trp/Glu333Ser | — |
| Lys326Ala/Glu333Ala | — |
| Lys326Met/Glu333Ser | — |
| Cys221Asp/Asp222Cys | — |
| Ser267Glu, His268Phe, and Ser324Thr | EFT |
| His268Phe and Ser324Thr | FT |
| Glu345Arg | Arg345 |

In some embodiments, the Fc domain binds to an Fcγ receptor. In some embodiments, the Fc domain binds to a human Fcγ receptor. The human Fcγ receptor may be FcγRI, FcγRIIA or FcγRIIIA. FcγRI is expressed on macrophages, neutrophils, eosinophils and DCs. FcγRIIA is expressed on macrophages, neutrophils, eosinophils, platelets, Langerhans cells and conventional DCs. FcγRIIIA is expressed on natural killer (NK) cells and macrophages. In this way, the bispecific binding agent widens the number of immune cells that are capable of coupling detection of necrotic cells to XP.

The linker functionally and structurally connects the F-actin binding moiety to the Fc receptor-binding moiety. In some embodiments, the linker is a peptide linker. In some embodiments, the peptide linker comprises the amino acid sequence GGGGSGGGGS. In some embodiments, the peptide linker comprises the amino acid sequence ARTGGGGSGGGGSDI. In some embodiments, the peptide linker comprises the amino acid sequence SGAGSNTSTSTGTSTSSSGPSSG. In some embodiments, the peptide linker comprises the amino acid sequence AEAAARAEAAARAEAAARAPPS. In some embodiments, the peptide linker comprises the amino acid sequence AEAAARAEAAARAEAAARAEAAARAAEAAARAEAAARAEAAARAEAAARAPPS. The linker may be any suitable linker that is sufficiently long to separate the F-actin binding moiety and the Fc receptor-binding moiety. In some embodiments, the linker comprises a serine-rich and/or glycine-rich peptide. In some embodiments, the linker is 5 to 100, 10 to 90, 20 to 80, 30 to 70, 40 to 60, or 45 to 50 amino acids in length. In some embodiments, the linker is 10 amino acids in length. In some embodiments, the linker is 15 amino acids in length. Preferably, the linker is non-immunogenic in humans.

In some embodiments, the linker is not IEGR. IEGR is a commonly-used commercial linker. Without wishing to be bound by any particular theory, the inventors hypothesise that IEGR may be too short and inflexible to allow the Fc receptor-binding moiety to bind an Fc receptor and the F-actin binding moiety to bind F-actin at the same time.

In some embodiments, the linker does not comprise a helical domain that extends unbroken across more than 60% of the length of the peptide linker. A helical secondary structure limits the structural freedom of a protein. Therefore, a linker with no more than 60% helical domain has increased flexibility. In some embodiments, the linker does not comprise a helical domain that extends unbroken across more than 10%, 20%, 30%, 40% or 50% of the length of the peptide linker. In some embodiments, the linker comprises at least 10, at least 12, at least 14, at least 16, at least 18 or at least 20 amino acids that do not form part of a helical domain.

In some embodiments, the linker does not comprise the neck region of human DNGR-1. In some embodiments, the linker comprises the neck region of a mouse DNGR-1. In some embodiments, the linker comprises the neck region of mouse DNGR-1 isoform 4.

In some embodiments, the linker is a non-peptide linker. For example, the linker may be a disulphide linker; a homobifunctional sulfhydryl-reactive crosslinker; a homobifunctional amine-reactive crosslinker; or a heterobifunctional amine- or sulfhydryl-reactive crosslinker.

Multiple bispecific binding agents can bind simultaneously to a necrotic cell via the F-actin displayed by the necrotic cell. By necrotic cell it is meant a cell that has undergone (and is consequently dead) or is undergoing (but may not yet be dead) cell death whereby the membrane has been permeabilised or ruptured and/or there is extracellular exposure of the cytoskeleton. For example, the cell death mechanism may be necrosis. This binding of multiple binding agents brings multiple Fc receptors into close proximity to each other, allowing for efficient cross-linking of the Fc receptors. Activatory FcγRs possess a tyrosine motif in their intracellular portion, termed immunoreceptor tyrosine-based activation motif (ITAM). This cross-linking triggers phosphorylation of the ITAM sequences by Src kinases, which allows recruitment of Src homology 2 (SH2) containing signaling molecules, including Spleen tyrosine kinase (Syk) kinase. This triggers a range of downstream processes, including phagocytosis, MHC class II presentation and cell activation. It is also hypothesised to trigger endosomal or phagosomal rupture and release of cellular components from the necrotic cell (necrotic cell debris) into the cytosol of the Fc receptor-expressing cell. The necrotic cell debris may include proteins that enter the MHC class I processing and presentation pathway, thereby allowing XP of the necrotic cell debris as a necrotic cell antigen. The necrotic cell antigens that are cross-presented on MHC class I (and presented on MHC class II) may be antigens that are not F-actin. The necrotic cell debris may also include nucleic acids that then trigger innate immune signalling pathways in endosomes and in the cytosol of the Fc receptor-expressing cell and that lead to induction of cytokines such as type I interferons.

In some embodiments, the bispecific binding agent is an antibody that binds actin and binds an Fc receptor, wherein the antibody does not block interaction of F-actin with DNGR-1. In some embodiments, the antibody only binds F-actin and does not bind G-actin. In some embodiments, the antibody has greater specificity for F-actin relative to G-actin. In some embodiments, the specificity for F-actin is between 2- and 100-fold greater than for G-actin. In some embodiments, the specificity for F-actin is between 2- and 10-fold greater than for G-actin. In some embodiments, the specificity for F-actin is at least 2-fold greater than for G-actin. In some embodiments, the specificity for F-actin is at least 5-fold greater than for G-actin. In some embodiments, the specificity for F-actin is at least 10-fold greater than for G-actin. In some embodiments, the specificity for F-actin is at least 100-fold greater than for G-actin. Specificity is explicitly intended to encompass affinity and/or avidity.

In some embodiments, the bispecific binding agent can be a fusion protein. In some embodiments, the F-actin binding moiety is selected from the group consisting of an aptamer, an affimer, a target-binding domain of an antibody and the C-type lectin-like domain (CTLD) of DNGR-1.

For example, in some embodiments, the F-actin binding moiety is an Fv domain against actin, and the Fc-receptor binding moiety is an Fc domain. In some embodiments, the F-actin binding moiety is the CTLD of human DNGR-1 and the Fc-receptor binding moiety is an Fc domain. In some embodiments, the F-actin binding moiety is an aptamer and the Fc-receptor binding moiety is an Fc domain. In some preferred embodiments, the F-actin binding moiety is an affimer and the Fc receptor-binding moiety is an Fc domain. In some embodiments, the bispecific binding agent may be a bispecific antibody, comprising two target-binding domains, and optionally comprising an Fc domain. In some embodiments, one target-binding fragment binds F-actin and the other target-binding fragment binds an Fc receptor. In some embodiments, the F-actin binding moiety is an affimer that binds actin. In some embodiments, the affimer is fused to the Fc receptor-binding moiety, optionally via a linker, via the N-terminus of the affimer. In some embodiments, the affimer is fused to the Fc receptor-binding moiety, optionally via a linker, via the C-terminus of the affimer. The terms “Affimer-Fc” and “Fc-Affimer” are used interchangeably herein. In some embodiments, the F-actin binding moiety is specific for F-actin. In some embodiments, the F-actin binding moiety binds both F-actin and G-actin. For the avoidance of doubt, the invention does not encompass bispecific binding agents which bind to G-actin but do not bind to F-actin.

In other aspects, the Fc receptor-binding moiety is selected from the group consisting of an aptamer, an affimer, and a target-binding domain of an antibody. The target-binding domain of an antibody may be a Fab fragment, a single chain Fc fragment (ScFv), a single domain antibody (sdAb), or a variable domain (Fv). For example, in some embodiments, the F-actin binding moiety is an affimer and the Fc-receptor binding moiety is a ScFv.

In some embodiments of the first aspect of the invention, the use in medicine comprises cancer therapy. In some of these embodiments, the bispecific binding agent is administered in combination with an immunogenic cancer treatment. In some embodiments, the immunogenic cancer treatment is radiotherapy or immunogenic chemotherapy. In some embodiments, the immunogenic cancer treatment is comprises a checkpoint inhibitor. In some embodiments, the bispecific binding agent is administered to the patient intratumorally (IT), intramuscularly (IM) or intravenously (IV).

In some embodiments, the cancer is characterised by F-actin exposure on necrotic cancer cells.

In some embodiments, the F-actin-binding moiety binds to F-actin on a macromolecular complex comprising F-actin and an antigen. The macromolecular complex may be a necrotic cell, a cancer cell, a necrotic cancer cell or a complex, e.g. cellular debris, derived therefrom. In some embodiments, the Fc receptor-binding moiety binds to an Fcγ receptor on a cell. In some embodiments, binding of the F-actin binding moiety to F-actin and binding of the Fc receptor-binding moiety to an Fc receptor occurs simultaneously. In some embodiments, this simultaneous binding leads to cross-presentation of the antigen by the Fcγ receptor-expressing cell. In some embodiments, the simultaneous binding leads to MHC class II presentation of the antigen by the Fcγ receptor-expressing cell. In some embodiments, the simultaneous binding leads to MHC Class II expression by the Fcγ receptor-expressing cell. In some embodiments, the simultaneous binding leads to presentation of exogenous antigens by MHC Class II. In some embodiments, the simultaneous binding leads to XP of MHC Class I antigens. In some embodiments, the simultaneous binding leads to phagocytosis of the macromolecular complex by the Fcγ receptor-expressing cell. In some embodiments, the simultaneous binding leads to activation of the Fcγ receptor-expressing cell. In some embodiments, the simultaneous binding leads to induction of type I interferon (IFN) expression. In some embodiments, the simultaneous binding leads to delivery of the macromolecular complex comprising F-actin and an antigen to the cytosol of the Fcγ receptor-expressing cell. In some embodiments, the Fcγ receptor-expressing cell is an antigen presenting cell (APC).

In some embodiments, the macromolecular complex is a necrotic cell and the antigen is a necrotic cell-associated antigen. In some embodiments, the macromolecular complex is a cancer cell and the antigen is a tumour antigen. In some embodiments, the antigen is not F-actin. In some embodiments, the use in medicine comprises treating a viral infection characterised by F-actin exposure on necrotic cells. In some embodiments, the antigen is a viral antigen. For the avoidance of doubt, the viral antigen is not F-actin.

In some embodiments, the Fc receptor-binding moiety has greater specificity for inhibitory Fc receptors relative to activatory receptors. In these embodiments, the bispecific binding agent may be for use in a method of treating an autoimmune disease.

In a second aspect, the invention provides a bispecific binding agent comprising an F-actin binding moiety and an Fc receptor-binding moiety, wherein the bispecific binding agent is capable of simultaneously (i) binding F-actin via the F-actin binding moiety and (ii) binding an Fc receptor via the Fc receptor-binding moiety and wherein (a) the F-actin binding moiety is the CTLD of human DNGR-1; or (b) the F-actin binding moiety is an affimer specific for F-actin; and/or (c) the Fc receptor-binding moiety preferentially binds an activatory Fc receptor relative to an inhibitory Fc receptor and the F-actin binding moiety has greater specificity for F-actin relative to G-actin. The CTLD of human DNGR-1 is specific for F-actin. The Fc receptor-binding moiety is specific for an Fc receptor. Preferably, the Fc receptor-binding moiety is an Fc domain, and the Fc domain comprises an amino acid sequence that has been mutated from that of a wild type Fc domain such that the mutated Fc domain binds to an Fc receptor with higher affinity and/or avidity than the affinity and/or avidity of the wild type Fc domain for the Fc receptor.

In some embodiments, the F-actin binding moiety has greater specificity for F-actin relative to G-actin. In some embodiments, the specificity for F-actin is between 2- and 100-fold greater than for G-actin. In some embodiments, the specificity for F-actin is between 2- and 10-fold greater than for G-actin. In some embodiments, the specificity for F-actin is at least 2-fold greater than for G-actin. In some embodiments, the specificity for F-actin is at least 5-fold greater than for G-actin. In some embodiments, the specificity for F-actin is at least 10-fold greater than for G-actin. In some embodiments, the specificity for F-actin is at least 100-fold greater than for G-actin. Specificity is explicitly intended to encompass affinity and/or avidity.

Unless otherwise stated, the Fc moiety is the same species as the C9 (DNGR-1) moiety, e.g., mC9-Fc comprises a murine C9 and a murine Fc moiety; and hC9-Fc comprises a human C9 and a human Fc moiety. In some specific embodiments, the Fc moiety and the C9 moiety may be from different species, e.g., a human C9 moiety and a murine Fc domain. The terms “mC9-mFc”, “mFc-mC9”, “mC9-Fc” and “mFc-C9” are used interchangeably herein.

In some embodiments, the F-actin binding moiety is linked to the Fc receptor-binding moiety via a linker that allows the Fc receptor-binding moiety to bind an Fc receptor and allows the F-actin binding moiety to bind F-actin at the same time. In some embodiments, the F-actin binding moiety is the CTLD of human DNGR-1. DNGR-1 is a type II membrane protein, which means that its orientation is reversed relative to type I membrane proteins, with the C-terminal domain targeted to the ER lumen. Therefore, in some embodiments, the CTLD of DNGR-1 may be fused to the Fc receptor-binding moiety via the DNGR-1 C-terminus, not its N-terminus. A linker may be advantageous in these embodiments to provide sufficient flexibility and/or distance between the Fc receptor-binding moiety and the CTLD of DNGR-1. In other embodiments, the CTLD of DNGR-1 may be fused to the Fc receptor-binding moiety via the DNGR-1 N-terminus. In these embodiments, different linkers may be used relative to embodiments fused via the DNGR-1 C-terminus. The linker enables simultaneous binding of the two components of the construct, by providing sufficient flexibility and/or separation between the two components to enable activation of various immune responses. The inventors have discovered that the known DNGR-1-Fc constructs are functionally restricted. Without wishing to be bound by any particular theory, the inventors hypothesise this may be due to the short IEGR linker.

The CTLD of DNGR-1 specifically binds polymeric F-actin but not monomeric G-actin. Therefore, DNGR-1 differentiates between polymeric actin displayed on necrotic cells and monomeric G-actin.

In some embodiments, the Fc receptor-binding moiety is engineered to preferentially bind an activatory Fc receptor relative to an inhibitory Fc receptor. In some embodiments, the Fc receptor-binding moiety is engineered to preferentially bind an activatory Fcγ receptor relative to an inhibitory Fcγ receptor. In some embodiments, the specificity for an activatory Fc receptor is between 2- and 100-fold greater than for an inhibitory Fc receptor. In some embodiments, the specificity for an activatory Fc receptor is between 2- and 10-fold greater than for an inhibitory Fc receptor. In some embodiments, the specificity for an activatory Fc receptor is at least 2-fold greater than for an inhibitory Fc receptor. In some embodiments, the specificity for an activatory Fc receptor is at least 5-fold greater than for an inhibitory Fc receptor. In some embodiments, the specificity for an activatory Fc receptor is at least 10-fold greater than for an inhibitory Fc receptor. In some embodiments, the specificity for an activatory Fc receptor is at least 100-fold greater than for an inhibitory Fc receptor. Specificity is explicitly intended to encompass affinity and/or avidity.

In preferred embodiments, the Fc receptor-binding moiety comprises an Fc (fragment, crystallisable) domain. Fc domains are the components of Ig molecules which bind to Fc receptors.

In some embodiments, the Fc domain comprises an Fc domain of an immunoglobulin that is capable of binding and triggering activatory Fcγ receptors. Human activatory Fcγ receptors include FcγRI, FcγRIIA and FcγRIIIA. Murine activatory Fcγ receptors include FcγRI, FcγRIII and FcγRIV.

In some embodiments, the Fc domain is engineered to preferentially bind an activatory Fcγ receptor relative to an inhibitory Fcγ receptor.

In some embodiments, the Fc domain is a human Fc domain.

In some embodiments, the Fc domain is from the pro-inflammatory immunoglobulin human IgG1 or human IgG3. Human IgG1 Fc has higher affinity for activating receptors, particularly FcγRI and FcγRIIA (Castro-Dopico, 2019).

In some embodiments, the Fc domain is from the pro-inflammatory immunoglobulin murine IgG2a. Murine IgG2a has higher affinity for the activating receptors FcγRI and FcγRIV (Castro-Dopico, 2019), relative to inhibitory receptors.

In some embodiments, the Fc domain comprises an amino acid sequence that has been mutated from that of a wild type Fc domain such that an activatory Fc receptor binds to the mutated Fc domain with higher affinity than the affinity of the Fc receptor for the wild type Fc domain. In some embodiments, the mutant Fc domain is a GASDALIE mutant Fc domain, comprising mutations corresponding to G236A, S239D, A330L and I332E. This mutation has been shown to have an activating effect on hFcγRI, hFcγRIIA (R131 and H131), and hFcγRIIIA (V158 and F158) (Bruhns, 2015). In some embodiments, the mutant Fc domain is a GA mutant Fc domain, comprising a mutation corresponding to G236A. In some embodiments, the mutant Fc domain is an ALIE mutant Fc domain, comprising mutations corresponding to A330L and I332E. In some embodiments, the mutant Fc domain comprises a mutation selected from one or more of the mutations set out in Table 2 above, which is adapted from Table 1 of Saunders (2019), which is incorporated by reference herein in its entirety.

In some embodiments, the Fc domain binds to an Fcγ receptor. In some embodiments, the Fc domain binds to a human Fcγ receptor. The human Fcγ receptor may be FcγRI, FcγRIIA or FcγRIIIA. FcγRI is expressed on macrophages, neutrophils, eosinophils and DCs. FcγRIIA is expressed on macrophages, neutrophils, eosinophils, platelets, Langerhans cells and conventional DCs. FcγRIIIA is expressed on natural killer (NK) cells and macrophages. In this way, the bispecific binding agent widens the number of immune cells that are capable of coupling detection of necrotic cells to XP.

The linker functionally and structurally connects the F-actin binding moiety (e.g., the CTLD of human DNGR-1) to the Fc receptor-binding moiety. In some embodiments, the linker is a peptide linker. In some embodiments, the peptide linker comprises the amino acid sequence GGGGSGGGGS. In some embodiments, the peptide linker comprises the amino acid sequence ARTGGGGSGGGGSDI. In some embodiments, the peptide linker comprises the amino acid sequence SGAGSNTSTSTGTSTSSSGPSSG. In some embodiments, the peptide linker comprises the amino acid sequence AEAAARAEAAARAEAAARAPPS. In some embodiments, the peptide linker comprises the amino acid sequence AEAAARAEAAARAEAAARAEAAARAAEAAARAEAAARAEAAARAEAAARAPPS. The linker may be any suitable linker that is sufficiently long to distance the Fc receptor-binding moiety and the F-actin binding moiety. In some embodiments, the linker comprises a serine-rich and/or glycine-rich peptide. In some embodiments, the linker is 5 to 100, 10 to 90, 20 to 80, 30 to 70, 40 to 60, or 45 to 50 amino acids in length. In some embodiments, the linker is 10 amino acids in length. In some embodiments, the linker is 15 amino acids in length. Preferably, the linker is non-immunogenic in humans.

In some embodiments, the linker is not IEGR. IEGR is a commonly-used commercial linker. Without wishing to be bound by any particular theory, the inventors hypothesise IEGR may be too short and inflexible to allow the Fc receptor-binding moiety to bind an Fc receptor and the F-actin binding moiety (such as the CTLD of human DNGR-1) to bind F-actin at the same time.

In some embodiments, the linker does not comprise a helical domain that extends unbroken across more than 60% of the length of the peptide linker. A helical secondary structure limits the structural freedom of a protein. Therefore, a linker with no more than 60% helical domain has increased flexibility. In some embodiments, the linker does not comprise a helical domain that extends unbroken across more than 10%, 20%, 30%, 40% or 50% of the length of the peptide linker. In some embodiments, the linker comprises at least 10, at least 12, at least 14, at least 16, at least 18 or at least 20 amino acids that do not form part of a helical domain.

In some embodiments, the linker does not comprise the neck region of human DNGR-1. In some embodiments, the linker comprises the neck region of a mouse DNGR-1. In some embodiments, the linker comprises the neck region of mouse DNGR-1 isoform 4.

In some embodiments, the linker is a non-peptide linker. For example, the linker may be a disulphide linker; a homobifunctional sulfhydryl-reactive crosslinker; a homobifunctional amine-reactive crosslinker; or a heterobifunctional amine- or sulfhydryl-reactive crosslinker.

Multiple bispecific binding agents can bind simultaneously to a necrotic cell via the F-actin binding moiety, (for example the human DNGR-1 CTLD) binding to F-actin displayed by the necrotic cell. By necrotic cell it is meant a cell that has undergone necrosis (and is consequently dead), or a cell that is in the process of necrosis (but may not yet be dead). This binding of multiple binding agents brings multiple Fc receptors into close proximity to each other, allowing for efficient cross-linking of the Fc receptors. Activatory FcγRs possess a tyrosine motif in their intracellular portion, termed immunoreceptor tyrosine-based activation motif (ITAM). This cross-linking triggers phosphorylation of the ITAM sequences by Src kinases, which allows recruitment of Src homology 2 (SH2) containing signaling molecules, including Spleen tyrosine kinase (Syk) kinase. This triggers a range of downstream processes, including phagocytosis, MHC class II presentation and cell activation. It is also hypothesised to trigger endosomal or phagosomal rupture and release of cellular components from the necrotic cell (necrotic cell debris) into the cytosol of the Fc receptor-expressing cell. The necrotic cell debris may include proteins that enter the MHC class I processing and presentation pathway, thereby allowing XP of the necrotic cell debris as a necrotic cell antigen. The necrotic cell antigens that are cross-presented on MHC class I (and presented on MHC class II) may be antigens that are not F-actin. The necrotic cell debris may also include nucleic acids that then trigger innate immune signalling pathways in endosomes and in the cytosol of the Fc receptor-expressing cell and that lead to induction of cytokines such as type I interferons.

In some embodiments, the F-actin binding moiety binds to F-actin on a macromolecular complex comprising F-actin and an antigen. The macromolecular complex may be a necrotic cell, a cancer cell, or a necrotic cancer cell. In some embodiments, the Fc receptor-binding moiety binds to an Fcγ receptor on a cell. In some embodiments, binding of the F-actin binding moiety to F-actin and binding of the Fc receptor-binding moiety to an Fc receptor occurs simultaneously. In some embodiments, this simultaneous binding leads to cross-presentation of the antigen by the Fcγ receptor-expressing cell. In some embodiments, the simultaneous binding leads to MHC class II presentation of the antigen by the Fcγ receptor-expressing cell. In some embodiments, the simultaneous binding leads to MHC Class II expression by the Fcγ receptor-expressing cell. In some embodiments, the simultaneous binding leads to presentation of exogenous antigens by MHC Class II. In some embodiments, the simultaneous binding leads to XP of MHC Class I antigens. In some embodiments, the simultaneous binding leads to phagocytosis of the macromolecular complex by the Fcγ receptor-expressing cell. In some embodiments, the simultaneous binding leads to activation of the Fcγ receptor-expressing cell. In some embodiments, the simultaneous binding leads to induction of type I interferon (IFN) expression. In some embodiments, the simultaneous binding leads to delivery of the macromolecular complex comprising F-actin and an antigen to the cytosol of the Fcγ receptor-expressing cell. In some embodiments, the Fcγ receptor-expressing cell is an antigen presenting cell (APC).

In some embodiments, the macromolecular complex is a necrotic cell and the antigen is a necrotic cell-associated antigen. In some embodiments, the macromolecular complex is a cancer cell and the antigen is a tumour antigen. In some embodiments, the antigen is not F-actin.

In some embodiments, the bispecific binding agent is an antibody that binds actin and an Fc receptor, wherein the antibody does not block interaction of F-actin with DNGR-1. In some embodiments, the antibody only binds F-actin and does not bind G-actin. In some embodiments, the bispecific binding agent is an antibody that binds actin and binds an Fc receptor, wherein the antibody does not block interaction of F-actin with DNGR-1. In some embodiments, the antibody only binds F-actin and does not bind G-actin. In some embodiments, the antibody has greater specificity for F-actin relative to G-actin. In some embodiments, the specificity for F-actin is between 2- and 100-fold greater than for G-actin. In some embodiments, the specificity for F-actin is between 2- and 10-fold greater than for G-actin. In some embodiments, the specificity for F-actin is at least 2-fold greater than for G-actin. In some embodiments, the specificity for F-actin is at least 5-fold greater than for G-actin. In some embodiments, the specificity for F-actin is at least 10-fold greater than for G-actin. In some embodiments, the specificity for F-actin is at least 100-fold greater than for G-actin. Specificity is explicitly intended to encompass affinity and/or avidity.

In some embodiments, the bispecific binding agent can be a fusion protein. In some embodiments, the bispecific binding agent may be a bispecific antibody, with two target-binding domains and optionally an Fc domain. In some embodiments, one target-binding fragment binds F-actin and the other target-binding fragment binds an Fc receptor.

In some aspects the Fc receptor-binding moiety is selected from the group consisting of an aptamer, an affimer, and a target-binding domain of an antibody. The target-binding domain of an antibody may be a Fab fragment, a single chain Fc fragment (ScFv), a single domain antibody (sdAb), or a variable domain (Fv). For example, in some embodiments, the Fc-receptor binding moiety is a ScFv.

In some embodiments, the Fc receptor-binding moiety has greater specificity for inhibitory Fc receptors relative to activatory receptors. In these embodiments, the bispecific binding agent may be for use in a method of treating an autoimmune disease.

In a third aspect, the invention provides a method for identifying a target tumour antigen, the method comprising contacting the bispecific binding agent of any previous aspect of the invention with a tumour biopsy sample, and analysing peptide epitopes that are cross presented on MHC class I molecules on antigen presenting cells present in the sample. In some embodiments of the method, the method comprises identifying the immunodominant epitope of the target tumour antigen.

In a fourth aspect, the invention provides a nucleic acid encoding the bispecific binding agent of any previous aspect of the invention. In some embodiments, this nucleic acid forms part of a vector.

In a fifth aspect, the invention provides a cell comprising the nucleic acid encoding the bispecific binding agent.

In a sixth aspect, the invention provides a plasmid for making the bispecific binding agent of any of the previous aspects of the invention. In some embodiments, the plasmid comprises a pFUSEN plasmid backbone.

In other aspects of the invention, there are provided bispecific binding agents comprising the CTLD of human DNGR-1 and a “null” Fc domain which does not interact with an Fc receptor. This embodiment reduces XP by competitively inhibiting the interaction of F-actin on dead cells with DNGR-1 on DCs. Thus, this embodiment has application in the treatment of autoimmune diseases. In some embodiments, the “null” Fc domain may comprise the mutations L234A, L235A, and P329G (LALA-PG). In some embodiments, the “null” Fc domain may comprise the mutation N297A.

In related aspects of the invention, there is provided an antibody comprising an F-actin binding moiety and an Fc receptor binding moiety, wherein the antibody is capable of simultaneously (i) binding F-actin via the F-actin binding moiety; and (ii) binding an Fc receptor-binding moiety via the Fc receptor binding moiety.

### Sequences

### Mouse DNGR-1 (CLEC9A) Isoform 1

The full-length amino acid sequence of mouse DNGR-1 (CLEC9A) isoform 1 is available on the public protein databases, e.g. on the UniProt database with identifier Q8BRU4-1, and is provided here by SEQ ID NO: 1:

(SEQ ID NO: 1)
MHAEEIYTSLQWDIPTSEASQKCQSPSKCSGAWCVVTMISCVVCM
GLLATSIFLGIKFFQ VSSLVLEQQERLIQQDTALVNLTQWQRKY
TLEYCQALLQRSLHSGSDCSPCPHNWIQNGKSCYYVFERWEMWNI
SKKSCLKEGASLFQIDSKEEMEFISSIGKLKGGNKYWVGVFQDGI
SGSWFWEDGSSPLSDLLPAERQRSAGQICGYLKDSTLISDKCDSW
KYFICEKKAFGSCI

### The Neck Domain of Mouse DNGR-1 Isoform 1

The part of the amino acid sequence of mouse DNGR-1 isoform 1 that is the neck domain is set forth in SEQ ID NO:2:

(SEQ ID NO: 2)
KFFQVSSLVLEQQERLIQQDTALVNLTQWQRKYTLEYCQALLQRS
LHSGSDCS

### Mouse DNGR-1 (CLEC9A) Isoform 4

Mouse DNGR-1 is a type II transmembrane protein. The full-length amino acid sequence of mouse DNGR-1 (CLEC9A) is available on the public protein databases, e.g. on the NCBI database with identifier NP_001192292.1, and is provided here by SEQ ID NO:3:

(SEQ ID NO: 3)
MHAEEIYTSLQWDIPTSEASQKCQSPSKCSGAWCVVTMISCVVCM
GLLATSIFLGIKFFQVSSLVLEQQERLIQQDTALVNLTQWQRKYT
LEYCQALLQRSLHSGTDASTGPVLLTSPQMVPQTLDSKETGSDCS
PCPHNWIQNGKSCYYVFERWEMWNISKKSCLKEGASLFQIDSKEE
MEFISSIGKLKGGNKYWVGVFQDGISGSWFWEDGSSPLSDLLPAE
RQRSAGQICGYLKDSTLISDKCDSWKYFICEKKAFGSCI

### The Neck Domain of Mouse DNGR-1 Isoform 4

The part of the amino acid sequence of mouse DNGR-1 isoform 4 that is the neck domain is set forth in SEQ ID NO:4:

(SEQ ID NO: 4)
KFFQVSSLVLEQQERLIQQDTALVNLTQWQRKYTLEYCQALLQRS
LHSGTDASTGPVLLTSPQMVPQTLDSKETGSDCS

### The C-Type Lectin Domain (CTLD) of Mouse DNGR-1

The part of the amino acid sequence of mouse DNGR-1 that is the CTLD is set forth in SEQ ID NO:5:

(SEQ ID NO: 5)
PCPHNWIQNGKSCYYVFERWEMWNISKKSCLKEGASLFQIDSKEEMEFI
SSIGKLKGGNKYWVGVFQDGISGSWFWEDGSSPLSDLLPAERQRSAGQI
CGYLKDSTLISDKCDSWKYFICEKKA

### Human DNGR-1 (CLEC9A)

Human DNGR-1 is a type II transmembrane protein. The full-length amino acid sequence of human DNGR-1 (CLEC9A) is available on the public protein databases, e.g. on the NCBI database with identifier NP_997228.1, and is provided here by SEQ ID NO:6:

(SEQ ID NO: 6)
MHEEEIYTSLQWDSPAPDTYQKCLSSNKCSGACCLVMVISCVFCMGLLT
ASIFLGVKLLQVSTIAMQQQEKLIQQERALLNFTEWKRSCALQMKYCQA
FMQNSLSSAHNSSPCPNNWIQNRESCYYVSEIWSIWHTSQENCLKEGST
LLQIESKEEMDFITGSLRKIKGSYDYWVGLSQDGHSGRWLWQDGSSPSP
GLLPAERSQSANQVCGYVKSNSLLSSNCSTWKYFICEKYALRSSV

### The Neck Domain of Human DNGR-1

The part of the amino acid sequence of human DNGR-1 that is the neck domain is set forth in SEQ ID NO: 7:

(SEQ ID NO: 7)
KLLQVSTIAMQQQEKLIQQERALLNFTEWKRSCALQMKYCQAFMQNSLS
SAHNSS

The C-type lectin domain (CTLD) of human DNGR-1

The part of the amino acid sequence of human DNGR-1 that is the CTLD is set forth in SEQ ID NO:8:

(SEQ ID NO: 8)
PCPNNWIQNRESCYYVSEIWSIWHTSQENCLKEGSTLLQIESKEEMDFI
TGSLRKIKGSYDYWVGLSQDGHSGRWLWQDGSSPSPGLLPAERSQSANQ
VCGYVKSNSLLSSNCSTWKYFICEKYA

### Human IgG1 Fc Domain

The amino acid sequence of the human IgG1 Fc domain is set forth in SEQ ID NO:9:

(SEQ ID NO: 9)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPG

### Human IgG1 Fc Domain GASDALIE Mutant

The amino acid sequence of the human IgG1 Fc domain GASDALIE mutant is set forth in SEQ ID NO:10:

(SEQ ID NO: 10)
DKTHTCPPCPAPELLAGPDVFLFPPKPKDTLMISRTPEVTCVVVDVSHE
DPEVKFNWYVDGVEVHNAKTKPREEQYNSTYRVVSVLTVLHQDWLNGKE
YKCKVSNKALPLPEEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPG

### Human IgG3 Fc Domain

The amino acid sequence of the human IgG3 Fc domain is set forth in SEQ ID NO:11:

(SEQ ID NO: 11)
TPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHE
DPEVQFKWYVDGVEVHNAKTKPREEQYNSTFRVVSVLTVLHQDWLNGKE
YKCKVSNKALPAPIEKTISKTKGQPREPQVYTLPPSREEMTKNQVSLTC
LVKGFYPSDIAVEWESSGQPENNYNTTPPMLDSDGSFFLYSKLTVDKSR
WQQGNIFSCSVMHEALHNRFTQKSLSLSPGK

### Mouse IgG2a Fc Domain

The amino acid sequence of the mouse IgG2a Fc domain is set forth in SEQ ID NO:12:

(SEQ ID NO: 12)
TIKPCPPCKCPAPNLLGGPSVFIFPPKIKDVLMISLSPIVTCVVVDVSE
DDPDVQISWFVNNVEVHTAQTQTHREDYNSTLRVVSALPIQHQDWMSGK
EFKCKVNNKDLPAPIERTISKPKGSVRAPQVYVLPPPEEEMTKKQVTLT
CMVTDFMPEDIYVEWTNNGKTELNYKNTEPVLDSDGSYFMYSKLRVEKK
NWVERNSYSCSVVHEGLHNHHTTKSFSRTPG

In-House mC9-mFc and Mutants
WT mC9-mFc

The amino acid sequence of the of the in-house WT mC9-mFc construct is set forth in SEQ ID NO:13:

(SEQ ID NO: 13)
MYRMQLLSCIALSLALVTNSAPLEPRGPTIKPCPPCKCPAPNLLGGPSV
FIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISK
PKGSVRAPQVYVLPPPEEEMTKKQVTLTCMVTDFMPEDIYVEWTNNGKT
ELNYKNTEPVLDSDGSYFMYSKLRVEKKNWVERNSYSCSVVHEGLHNHH
TTKSFSRTPGARTGGGGSGGGGSDIKFFQVSSLVLEQQERLIQQDTALV
NLTQWQRKYTLEYCQALLQRSLHSGTDASTGPVLLTSPQMVPQTLDSKE
TGSDCSPCPHNWIQNGKSCYYVFERWEMWNISKKSCLKEGASLFQIDSK
EEMEFISSIGKLKGGNKYWVGVFQDGISGSWFWEDGSSPLSDLLPAERQ
RSAGQICGYLKDSTLISDKCDSWKYFICEKKAFGSCI

N297A mC9-mFc

The amino acid sequence of the in-house N297A mutant mC9-mFc construct is set forth in SEQ ID NO: 14:

(SEQ ID NO: 14)
MYRMQLLSCIALSLALVTNSAPLEPRGPTIKPCPPCKCPAPNLLGGPSV
FIFPPKIKDVLMISLSPIVTCVVVDVSEDDPDVQISWFVNNVEVHTAQT
QTHREDYASTLRVVSALPIQHQDWMSGKEFKCKVNNKDLPAPIERTISK
PKGSVRAPQVYVLPPPEEEMTKKQVTLTCMVTDFMPEDIYVEWTNNGKT
ELNYKNTEPVLDSDGSYFMYSKLRVEKKNWVERNSYSCSVVHEGLHNHH
TTKSFSRTPGARTGGGGSGGGGSDIKFFQVSSLVLEQQERLIQQDTALV
NLTQWQRKYTLEYCQALLQRSLHSGTDASTGPVLLTSPQMVPQTLDSKE
TGSDCSPCPHNWIQNGKSCYYVFERWEMWNISKKSCLKEGASLFQIDSK
EEMEFISSIGKLKGGNKYWVGVFQDGISGSWFWEDGSSPLSDLLPAERQ
RSAGQICGYLKDSTLISDKCDSWKYFICEKKAFGSCI

2WA mC9-mFc

The amino acid sequence of the in-house 2WA mutant mC9-mFc construct is set forth in SEQ ID NO:15:

(SEQ ID NO: 15)
MYRMQLLSCIALSLALVTNSAPLEPRGPTIKPCPPCKCPAPNLLGGPSV
FIFPPKIKDVLMISLSPIVTCVVVDVSEDDPDVQISWFVNNVEVHTAQT
QTHREDYNSTLRVVSALPIQHQDWMSGKEFKCKVNNKDLPAPIERTISK
PKGSVRAPQVYVLPPPEEEMTKKQVTLTCMVTDFMPEDIYVEWTNNGKT
ELNYKNTEPVLDSDGSYFMYSKLRVEKKNWVERNSYSCSVVHEGLHNHH
TTKSFSRTPGARTGGGGSGGGGSDIKFFQVSSLVLEQQERLIQQDTALV
NLTQWQRKYTLEYCQALLQRSLHSGTDASTGPVLLTSPQMVPQTLDSKE
TGSDCSPCPHNWIQNGKSCYYVFERAEMWNISKKSCLKEGASLFQIDSK
EEMEFISSIGKLKGGNKYWVGVFQDGISGSWFWEDGSSPLSDLLPAERQ
RSAGQICGYLKDSTLISDKCDSAKYFICEKKAFGSCI

### Affimer-Fc Sequences

### Affimer 6

The amino acid sequence of the in-house affimer 6-mFc construct is set forth in SEQ ID NO:16:

(SEQ ID NO: 16)
MYRMQLLSCIALSLALVTNSTIKPCPPCKCPAPNLLGGPSVFIFPPKIK
DVLMISLSPIVTCVVVDVSEDDPDVQISWFVNNVEVHTAQTQTHREDYN
STLRVVSALPIQHQDWMSGKEFKCKVNNKDLPAPIERTISKPKGSVRAP
QVYVLPPPEEEMTKKQVTLTCMVTDFMPEDIYVEWTNNGKTELNYKNTE
PVLDSDGSYFMYSKLRVEKKNWVERNSYSCSWHEGLHNHHTTKSFSRTP
GARTGGGGGGGGSGGGGSDIASNSLEIEELARFAVDEHNKKENALLEFV
RVVKAKEQSSVPHWWWTTMYYLTLEAKDGGKKKLYEAKVWVKRDPNMIF
KINFKELQEFKPVGDAAGSTGSR

### Control Affimer

The amino acid sequence of the control affimer-Fc construct is set forth in SEQ ID NO: 17:

(SEQ ID NO: 17)
MYRMQLLSCIALSLALVTNSTIKPCPPCKCPAPNLLGGPSVFIFPPKIK
DVLMISLSPIVTCVVVDVSEDDPDVQISWFVNNVEVHTAQTQTHREDYN
STLRVVSALPIQHQDWMSGKEFKCKVNNKDLPAPIERTISKPKGSVRAP
QVYVLPPPEEEMTKKQVTLTCMVTDFMPEDIYVEWTNNGKTELNYKNTE
PVLDSDGSYFMYSKLRVEKKNWVERNSYSCSWHEGLHNHHTTKSFSRTP
GARTGGGGSGGGGSGGGGSDIASNSLEIEELARFAVDEHNKKENALLEF
VRVVKAKEQVVAGTMYYLTLEAKDGGKKKLYEAKVWVKPWENFKELQEF
KPVGDA

### Affimer 14

The amino acid sequence of the affimer 14-mFc construct is set forth in SEQ ID NO: 18:

(SEQ ID NO: 18)
MYRMQLLSCIALSLALVTNSTIKPCPPCKCPAPNLLGGPSVFIFPPKIK
DVLMISLSPIVTCVVVDVSEDDPDVQISWFVNNVEVHTAQTQTHREDYN
STLRVVSALPIQHQDWMSGKEFKCKVNNKDLPAPIERTISKPKGSVRAP
QVYVLPPPEEEMTKKQVTLTCMVTDFMPEDIYVEWTNNGKTELNYKNTE
PVLDSDGSYFMYSKLRVEKKNWVERNSYSCSWHEGLHNHHTTKSFSRTP
GARTGGGGSGGGGSGGGGSDIASNSLEIEELARFAVDEHNKKENALLEF
VRVVKAKEQSDTPHWWWTTMYYLTLEAKDGGKKKLYEAKVWVKESPVHP
KRLNFKDLQEFKPVGDAAGSTGSR

### Affimer 24

The amino acid sequence of the affimer 24-mFc construct is set forth in SEQ ID NO:19:

(SEQ ID NO: 19)
MYRMQLLSCIALSLALVTNSTIKPCPPCKCPAPNLLGGPSVFIFPPKIK
DVLMISLSPIVTCVVVDVSEDDPDVQISWFVNNVEVHTAQTQTHREDYN
STLRVVSALPIQHQDWMSGKEFKCKVNNKDLPAPIERTISKPKGSVRAP
QVYVLPPPEEEMTKKQVTLTCMVTDFMPEDIYVEWTNNGKTELNYKNTE
PVLDSDGSYFMYSKLRVEKKNWVERNSYSCSWHEGLHNHHTTKSFSRTP
GARTGGGGSGGGGSGGGGSDIASNSLEIEELARFAVDEHNKKENALLEF
VRVVKAKEQMDMIGEYVSTMYYLTLEAKDGGKKKLYEAKVWVKGWMPLY
SRQNFKELQEFKPVGDAAGSTGSR

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. mDNGR-1-mFc (mC9-Fc) construct can trigger Fc receptors and bind to F-actin. (A) NUNC MaxiSorp™ plates (Thermo Fisher) were coated overnight with mouse IgG1 (mIgG1), mIgG2a, or a mouse DNGR-1-Fc construct (mC9-Fc) obtained from R&D Systems (#6776-CL). RAW264.7 macrophages were added to the plate for 16 hours, and the supernatants assessed for TNF production. (B & C) As in A, R&D mC9-Fc, WT in-house mC9-Fc and 2WA mutant were equally able to trigger Fcγ receptors, as shown by measuring (B) TNF and (C) CXCL2 by ELISA. The N297A mutant was unable to trigger Fcγ receptors. (D & E) Binding of mC9-Fc (R&D and in-house) to F-actin was investigated by incubating the mC9-Fc constructs with (D) F-actin/myosin-II (FM) beads and (E) UV-treated 5555 dead tumour cells, for 1 hour, followed by detection of binding using anti-DNGR-1 lgG PE. The WT constructs and the N297A mutant were able to bind to FM beads and dead cells, whereas the 2WA mutant that is abolished for binding to F-actin was not.

FIG. 2. mC9-Fc can induce phagocytosis by various myeloid cells. Phagocytosis of fluorescent FM beads or of Cell Tracker Deep Red (CT-DR)-labelled UV-treated 5555 dead tumour cells by various myeloid cells in the presence or absence of mC9-Fc was measured by flow cytometry at several timepoints. (A & B) mC9-Fc induced phagocytosis of FM beads by RAW264.7 macrophages. (C-G) mC9-Fc induced phagocytosis of UV-treated 5555 dead tumour cells by (C & D) RAW264.7 macrophages; (E & F) primary FLT3L-derived bone marrow DC (FLT3L-DC) and (G) GM-CSF-derived bone marrow cells (GMC).

FIG. 3. Cross-presentation. (A) Primary FLT3L-DC subsets were enriched to 90% purity (XCR1-enrichment for cDC1, CLEC10A enrichment for cDC2), primed with IFNα for 16 h, and incubated with OVA-soaked UV-5555 dead cells+/−mC9-Fc and OT-I CD8⁺ T cells. T cell-derived IFNγ was measured after 24 h. (B-D) T cell co-cultures were also performed with other FcγR-expressing myeloid cells and OVA-dead cells or OVA-FM-beads: (B) HoxB8 cDC2 cell line, (C) GMC and (D) RAW264.7.Clec9a.Kb cells. (E) The level of XP using the in-house R&D mC9-Fc, in-house WT mC9-Fc and 2WA and N297A mutants was also compared in FLT3L-DC. The in-house WT construct boosted XP to the same extent as the R&D construct by both XCR1-enriched (cDC1) and XCR1-depleted (non-cDC1) APCs isolated from IFNα-primed FLT3L-DC cultures.

FIG. 4. mC9-Fc restores XP in non-cDC1 to the same level as cDC1. The range of cells that can perform XP of dead cell antigens is increased using mC9-Fc. In the presence of mC9-Fc, XCR-1-depleted cells from FLT3L-DC cultures, termed non-cDC1 cells (enriched for cDC2), were able to perform XP of OVA-dead cells to the same level as XCR1-enriched cDC1 cells.

FIG. 5. Investigating the mechanism of action of mC9-Fc. (A) Investigating effect of endogenous DNGR-1 on mC9-Fc function. WT or Clec9a^{-/-} XCR-1-enriched FLT3L-cDC1 cells were incubated with OVA-soaked UV-treated 5555 dead cells and in-house mC9-Fc constructs (WT, 2WA and N297A mutants). After 3 h, OT-I T cells were added, and XP assessed by IFNγ production by T cells after 24 h. The function of mC9-Fc constructs was unaffected by loss of DNGR-1, and therefore mC9-Fc does not rely on endogenous DNGR-1 to function. (B) Investigation into effect of mC9-Fc on dead cell XP by endogenous DNGR-1 on cDC1 cells. The FcγR binding-null N297A mutant showed the same level of XP inhibition as that seen in the DNGR-1 KO cells incubated with or without the N297A mutant. Therefore, the mC9-Fc inhibits endogenous DNGR-1 binding to F-actin, and thus prevents downstream XP. (C) Investigating the role of endogenous DNGR-1 in the mechanism of action of mC9-Fc. The XP ability of WT cDC1 cells+WT in-house mC9-Fc was compared to the XP ability of DNGR-1 KO cDC1 cells+WT in-house mC9-Fc construct. A similar level of XP was obtained that was independent of the presence of endogenous DNGR-1. Thus, mC9-Fc does not depend on endogenous DNGR-1 for XP.

FIG. 6. Cross-presentation mediated by mC9-Fc occurs via the cytosolic pathway. Primary FLT3L-DCs were incubated in IFNα for 24 hours. They were then split into cDC1 (XCR1-enriched) and non-cDC1 (XCR1-depleted). Each group was incubated with OVA-soaked UV-treated 5555 dead cells in the presence or absence of lactacystin (a proteasome inhibitor) and leupeptin (a protease inhibitor) with either the in-house WT mC9-Fc or the 2WA mutant for 3 h before addition of OT-I T cells. The level of XP was assessed by measuring T cell-derived IFNγ. XP by mC9-Fc was inhibited by lactacystin, demonstrating a requirement on the cytosolic proteasome. Thus, mC9-Fc XP proceeds via the cytosolic pathway.

FIG. 7. sGSN attenuates mC9-Fc dead cell XP by primary DC cells. (A & B) Primary FLT3L-DCs were primed with IFNα for 24 hours and split into cDC1 (XCR1-enriched) and non-cDC1 (XCR1-depleted). These were then incubated with OVA-soaked UV-treated 5555 dead cells and either normal mouse serum or sGSN^{-/-} serum and either WT in-house mC9-Fc or the 2WA mutant for 3 h before addition of OT-I T cells. The level of XP was assessed by measuring T cell-derived IFNγ production. sGSN was shown to attenuate mC9-Fc dead cell XP by both cDC1 (A) and non-cDC1 (cDC2) (B). The presence of serum containing sGSN significantly impaired XP by cDC1 to OT-I CD8⁺ T cells in vitro in the presence and absence of mC9-Fc (A). In contrast, XP by non-cDC1 was only impaired in the presence of the WT mC9-Fc (B). Therefore, mC9-Fc XP is inhibited by sGSN in IFNα-primed cDC1 and cDC2.

FIG. 8. Effect of inhibitory Fcγ receptor IIB expression on mC9-Fc XP. Fcgr2b KO hematopoietic bone marrow cells were generated using CRISPR-Cas9, then differentiated into FLT3L-DC. Along with control FLT3L-DC, the KO DC were primed with IFNα for 24 hours and split into cDC1 (XCR1-enriched) or non-cDC1 (XCR1-depleted), as before. These were then incubated with OVA-soaked UV-treated 5555 dead cells and either WT in-house mC9-Fc or the 2WA mutant, followed by OT-I T cells after 3 h. The Fcgr2b KO had no effect on the ability of the WT mC9-Fc construct to perform XP of dead cells. Therefore, the expression of the inhibitory FcγRIIB receptor does not affect XP by mC9-Fc.

FIG. 9. Myeloid cell activation with DAMPs. FLT3L-DC were primed with IFNα for 24 hours and split into cDC1 (XCR1-enriched) or non-cDC1 (XCR1-depleted), then incubated with UV-treated 5555 dead cells+/−soaking in poly(I:C) and either WT in-house mC9-Fc or the 2WA mutant for 24 h. Supernatants from cDC treated with control or poly(I:C)-soaked dead cells+/−mC9-Fc were added to LL171 bioassay cells that contain an interferon stimulating response element (ISRE)-luciferase construct. Luciferase activity was measured after 6 h. No T cells were present in the cDC cultures. WT mC9-Fc led to a boost in type I IFN (IFNα/B) production, particularly by non-cDC1 (cDC2). Left-hand plots are control dead cells; right-hand plots are soaked in poly(I:C).

FIG. 10. mC9-Fc reduces tumour growth in vivo. The in-house mC9-Fc construct (WT and 2WA mutant) was administered intratumourally (50 μg in 50 μl) to WT C57BL/6 mice with MCA205 tumours, in combination with 2.5 mg/kg doxorubicin to induce immunogenic cell death. 1 dose of doxorubicin or PBS was administered (day 7), and 2 doses of mC9-Fc were administered (day 7 and 11). WT mC9-Fc delayed MCA205 tumour growth when administered with doxorubicin intratumourally.

FIG. 11. Preliminary experiments using hDNGR-1-hFc construct. (A) The commercially available recombinant Human CLEC9a Fc Chimera Protein (R&D Systems, 6049-CL) was incubated with UV-treated 5555 dead cells. The DNGR-1 CTLD of the human construct was able to bind to F-actin on UV-treated mouse 5555 dead cells. (B) RAW264.7 macrophages expressing mouse Fcγ receptors were added to a plate coated with the human DNGR-1-Fc construct (hC9-Fc), WT in-house mC9-Fc, or with human lgG for 24 h. Supernatants were assessed for TNF production. The human construct was not able to trigger the mouse Fc receptors on the RAW264.7 cells, but human IgG and mC9-Fc could. (C) Primary murine FLT3L-DCs were primed with IFNα for 24 hours and split into cDC1 (XCR1-enriched) and non-cDC1 (XCR1-depleted). These were then incubated with OVA-soaked UV-treated 5555 dead cells and either in-house mC9-Fc reagents (WT and 2WA) or hC9-Fc for 3 h before addition of OT-I T cells. The level of XP was assessed by measuring T cell-derived IFNγ production after 24 h. The human construct was not able to augment XP.

FIG. 12. Affimer-Fc binding properties. (A, B) Binding of mC9-Fc (in-house) and Affimer-Fc to F-actin was investigated by incubating the Fc-fusion proteins with UV-treated 5555 dead tumour cells, for 1 hour at equimolar concentrations. Binding was detected using anti-mouse IgG-A647 (GMFI). (C) NUNC MaxiSorp™ plates (Thermo Fisher) were coated overnight with Fc-fusion constructs. RAW264.7 macrophages were added to the plate for 16 hours, and the supernatants assessed for TNF production. Affimer-Fc and mC9-Fc were equally able to trigger Fcγ receptors, as shown by measuring TNF by ELISA. The N297A mutant was unable to trigger Fcγ receptors. (D & E) Cross-blocking of Affimer-Fc with mC9 was assessed by incubating UV-5555 dead cells with Fc-fusion proteins for 1h, followed by incubation with 1 g/ml mC9-FLAG for 30 min. mC9-Fc was used as a positive control for inhibition. Binding of fusion proteins and mC9-FLAG was detected using anti-mouse IgG and anti-FLAG IgG, respectively. (F) Inhibition of Affimer-Fc binding to dead cells by sGSN was investigated by incubating UV-5555 dead cells with 10 μg/ml sGSN for 1 h at 4° C., followed by incubation with 10 nM Affimer-Fc. Binding of all Affimer-Fc to dead cells was inhibited by sGSN.

FIG. 13. cDC1 represent a rare antigen presenting cell subset in tumours. (A, B) cDC frequency in human tumours (A) and murine MCA205 tumours (day 11) in WT C57BL/6 mice (B). Human cDC data derived from the pan-cancer human myeloid cell atlas (Cheng et al, 2021. panmyeloid.cancer-pku.cn).

FIG. 14. FcγR expression on intratumoural XCR1⁺ cDC1. FcγR expression profiling on XCR1+ cDC1 in MCA205 tumours on day 11 (top row) and splenic cDC1 (bottom row). Stains are compared to fluorescence minus one (FMO) control.

FIG. 15. FcγR expression on intratumoural CD172a⁺ cDC2. (A, B) FcγR expression profiling on CD172a⁺ cDC2 in MCA205 tumours on day 11 (A, top row) and splenic cDC2 (A, bottom row), or HoxB8-CDP-derived cDC2 (B). Stains are compared to fluorescence minus one (FMO) control.

FIG. 16. FcγR expression on intratumoural CD88⁺ MC. FcγR expression profiling on CD11c⁺ MHC-II⁺ CD88⁺ MCs in MCA205 tumours on day 11 (top row) or splenic monocytes (bottom row). Stains are compared to fluorescence minus one (FMO) control.

FIG. 17. mC9-Fc promotes dead cell phagocytosis by cDC1 and cDC2. (A, B) Phagocytosis of CT-DR-labelled UV-irradiated 5555 necrotic cells by FLT3L-cDC1 (A) and FLT3L-cDC2 (B), assessed by confocal microscopy.

FIG. 18. mC9-Fc promotes sensing of dead cell-associated nucleic acids. (A, B) IFNβ production by FLT3L-non-cDC1 (cDC2) treated with poly(I:C)-soaked necrotic cells in the presence of mC9-Fc fusion proteins for 24 h. WT mC9-Fc leads to a boost in IFNβ production. IFNβ production was assessed in control (A) or Mavs-targeted (B) non-cDC1, using CRISPR-Cas9 technology, with Mavs deletion having no effect.

FIG. 19. mC9-Fc reduces tumour growth in vivo. (A, B, C) WT mC9-Fc was administered intratumourally at 50 μg (A), 100 μg (B), or 200 μg (C) doses in 50 μl to WT C57BL/6 mice with MCA205 tumours. mC9-Fc was administered in combination with 2.5 mg/kg doxorubicin to induce immunogenic cell death. 1 dose of doxorubicin or PBS was administered (day 7), and 2 doses of mC9-Fc were administered (day 7 and 11). 50 μg 2WA mC9-Fc was used as a control. WT mC9-Fc delayed MCA205 tumour growth when administered with doxorubicin intratumourally, increasing effectiveness with dose escalation. Data pooled from 2 independent experiments. N=8-18 per group.

FIG. 20. mC9-Fc reduces tumour growth in vivo. WT or 2WA mC9-Fc was administered intratumourally at 50 μg in 50 μl to WT C57BL/6 mice with MCA205 tumours. mC9-Fc was administered in combination with localised X-ray radiotherapy (XRT) to induce immunogenic cell death. 1 round of XRT was performed (day 6), and 2 doses of mC9-Fc were administered (day 6 and 11). WT mC9-Fc delayed MCA205 tumour growth in combination with XRT. N=10 per group.

FIG. 21. mC9-hFc fusion protein can bind to dead cells and trigger human FcγR. (A) Binding of in-house mC9-hFc proteins (WT hFc, N297A hFc, GASDALIE hFc, 2WA hFc) to UV-treated 5555 dead tumour cells. Fusion proteins were incubated for 1 hour, followed by detection of binding using anti-mouse DNGR-1 lgG PE. Binding was compared to in-house mC9-mFc reagent. All constructs bind equally to dead cells, except 2WA hFc, where binding is abolished. (B) NUNC MaxiSorp™ plates (Thermo Fisher) were coated overnight with human IgG1 (hIgG1) or mC9-hFc proteins. M-CSF-derived bone marrow macrophages (BMDM) from hFcγR-transgenic mice were added to the plate for 16 hours, and the supernatants assessed for TNF production. All mC9-hFc fusion proteins trigger hFcγR effectively, except N297A, which shows attenuated activity.

FIG. 22. Cross-presentation by hFcγR APC. (A, B) IFNα-primed primary XCR1-enriched cDC1 (A, left), XCR1-depleted non-cDC1 (cDC2) (A, right), and GMC (B) from human FcγR-transgenic mice were incubated with OVA-soaked UV-5555 dead cells (1:1 fixed ratio)+/−mC9-hFc and OT-I CD8⁺ T cells. T cell-derived IFNγ was measured after 24 h. WT mC9-hFc boosted XP relative to untreated cells or 2WA mC9-hFc fusion protein in cDC2 or GMC.

FIG. 23. Anti-actin IgG2a promotes dead cell cross-presentation. (A) Serial dilutions of mouse anti-actin IgG2a (AC-40, Abcam) were incubated with UV-irradiated 5555 dead cells for 30 min. Binding was detected with goat anti-mouse IgG2a AF488 and compared to mFc-mC9. (B) Primary FLT3L CLEC10A-enriched cDC2 were primed with IFNα for 16 h prior to incubation with OVA-soaked UV-5555 dead cells+/−AC-40 (1:100) and OT-I CD8⁺ T cells. T cell-derived IFNγ was measured after 24 h.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Cross-presentation (XP) of necrotic cell antigens by cDC1 at sites of pathological cell death can prime cytotoxic CD8⁺ T cell responses. Therefore, promoting XP may enhance cytotoxic immune responses that combat diseases, such as cancer. cDC1 are specialised in XP of necrotic cell-associated antigens, in part attributable to their high expression of DNGR-1 (a.k.a. CLEC9A or C9) in both mice and humans, a receptor that binds to F-actin exposed by dying cells and signals to promote XP of antigens associated with necrotic cell corpses. However, DNGR-1 expression is highly restricted to cDC1, such that the number of cells that can cross-present necrotic cell antigens in this way is limited. In contrast, a wide array of different cell types express Fcγ receptors-cell surface receptors also capable of promoting XP of lgG-bound targets. By enlisting a DNGR-1-Fc (C9-Fc) construct, the inventors have enabled XP to be triggered by a wider range of APCs, increasing the sensitivity and ability of the immune system to detect pathological cell death. In addition, the inventors have found that a DNGR-1-Fc construct triggers in addition to XP a range of downstream processes on the Fc receptor-expressing cell, including more efficient phagocytosis of necrotic cell debris by Fcγ receptor-expressing cells, more efficient delivery of necrotic cell debris to the cytosol of Fcγ receptor-expressing cells, MHC class II presentation of necrotic cell-associated antigens, triggering of innate immune receptors by necrotic cell-derived nucleic acids and activation of Fcγ receptor-expressing cells.

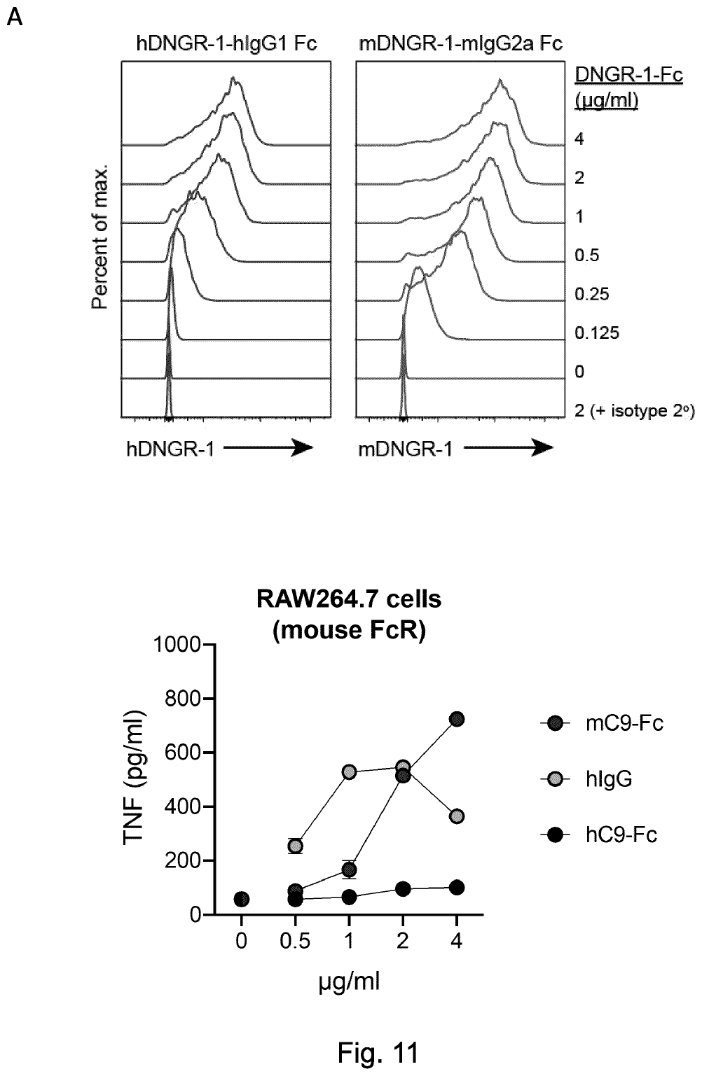

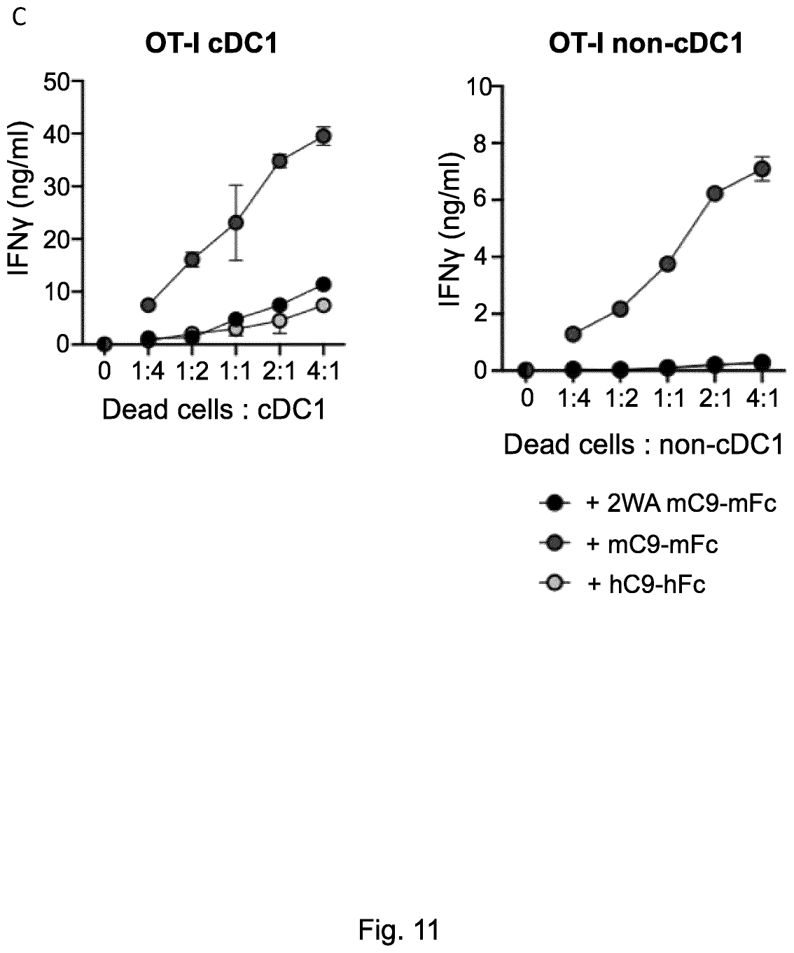

The inventors have surprisingly found that a fully human DNGR-1-Fc (hC9-Fc) fusion construct is not capable of engaging Fcγ receptors or inducing XP of necrotic cell antigens (as shown in FIG. 11). This finding is surprising because the fully mouse fusion construct (mouse DNGR-1-mouse Fc or mC9-Fc) was shown to mediate necrotic cell phagocytosis and XP (as shown in Examples 2, 3, 5 and 8). Without wishing to be bound by any particular theory, the inventors hypothesise this lack of function may be due to reduced flexibility compared to the mouse construct, which has a longer DNGR-1 neck domain. They further hypothesised that using a longer linker between the Fcγ receptor-binding moiety and the CTLD of human DNGR-1 may enable Fcγ receptor engagement and induction of XP of necrotic cell antigens. In this way, the longer linker could emulate the function of the longer neck region in the long DNGR-1 mouse isoform and enable the fully human DNGR-1-Fc fusion construct to bind F-actin and an Fc receptor simultaneously.

### Binding Agents

The bispecific binding agent may be an “affinity protein” or “engineered protein scaffold”. These can routinely be tailored for affinity against a particular target. They are typically based on a non-immunoglobulin scaffold protein with a conformationally stable or rigid core, which has been modified to have affinity for the target. Such molecules are clearly envisaged for use as binding agents in the present invention.

Other types of binding agent capable of binding specifically to F-actin or to an Fc receptor may also be used, such as nucleic acids (e.g. aptamers), carbohydrates (e.g. oligo- or polysaccharide), small molecules, etc.

The F-actin binding moiety and the Fc receptor binding moiety may each be the same or different type of binding agent.

### Fc Domains

The Fc domain (fragment, crystallizable region) is the region of an antibody that allows it to interact with other proteins, including Fc receptors and some proteins of the complement system. Therefore, the Fc domain allows antibodies to activate the immune system. IgG Fc domains contain a highly conserved N-glycosylation site, which is essential for Fcγ receptor-mediated activity. Fc domains can be mutated to alter the binding characteristics with their Fc receptors.

### Anti-F-Actin Antibodies

The bispecific binding agent may be an antibody. The bispecific binding agent may be an antibody with a target-binding fragment specific for actin and an Fc domain. The target-binding fragment may be specific for F-actin. The target-binding fragment may bind to both F-actin and G-actin. The bispecific binding agent may be a bispecific antibody, with a target-binding fragment specific for F-actin and a target-binding fragment specific for an Fc receptor. The antibody may be a target-binding fragment of an antibody (for example a Fab fragment) or a synthetic antibody fragment (for example a single chain Fv fragment [ScFv] or single-domain antibody/nanobody). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen. Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel DL (2002). “Recombinant monoclonal antibody technology”. Schmitz U, Versmold A, Kaufmann P, Frank HG (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799)).

Polyclonal antibodies are useful in the methods of the invention. Monospecific polyclonal antibodies are preferred. Suitable polyclonal antibodies can be prepared using methods well known in the art.

Fragments of antibodies, such as Fab and Fab2 fragments may also be used as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are linked via a flexible oligopeptide (Bird et al (1988) Science 242, 423; Huston et al (1988) Proc. Natl. Acad. Sd. USA 85, 5879) and single domain antibodies (sdAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the VH and VL partner domains are covalently linked, e.g. directly, by a peptide or by a flexible oligopeptide. Fab, Fv, ScFv and sdAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and sdAb fragments are monovalent, having only one antigen combining site. Synthetic antibodies which bind to a target discussed herein may also be made using phage display technology as is well known in the art (e.g. see “Phage display: a molecular tool for the generation of antibodies—a review”. Placenta. 21 Suppl A: S106-12. Helen E. Chadd and Steven M. Chamow; “Phage antibodies: filamentous phage displaying antibody variable domains”. Nature 348 (6301): 552-554).

### Affimers®

The bispecific binding agent may be an Affimer®. Affimers® are small binding proteins which mimic antibodies in terms of molecular recognition characteristics. They exhibit higher stability in comparison to antibodies. They are recombinant proteins which can be engineered to bind a target of interest. All Affimers® consist of an alpha-helix on top of an anti-parallel beta-sheet, as well as two peptide loops. These peptide loops can be randomised to bind to the desired target. The affimer may have affinity and/or avidity for both F-actin and G-actin. The affimer may be specific for F-actin. The affimer may be fused to the Fc receptor-binding moiety, optionally via a linker, via the N- or C-terminus of the affimer. The terms “Affimer-Fc” and “Fc-Affimer” are used interchangeably herein.

### Antigens

The antigen may be any protein or fragment thereof against which it is desirable to raise an immune response, in particular a CTL response, but also a Th17 response or a Treg response. These may include antigens associated with, expressed by, displayed on, or secreted by cells against which it is desirable to stimulate a CTL response, including cancer cells and cells containing intracellular pathogens or parasites. For example, the antigen may be, or may comprise, an epitope peptide from a protein expressed by an intracellular pathogen or parasite (such as a viral protein) or from a protein expressed by a cancer or tumour cell. Thus, the antigen may be a tumour-specific antigen. The term “tumour-specific” antigen should not be interpreted as being restricted to antigens from solid tumours, but to encompass antigens expressed specifically (or preferentially) by any cancerous, transformed or malignant cell.

The antigen may be a necrotic cell-associated antigen. By necrotic cell we mean a cell that has undergone necrosis, or a cell that is in the process of necrosis. A necrotic cell-associated antigen may be necrotic cell debris (cellular components released from a necrotic cell during endosomal or phagosomal rupture). The necrotic cell-associated antigen may be an antigen that is not F-actin.

### Fcγ Receptors

Preferably, the Fc receptor binding moiety of the bispecific binding agent binds to an activatory Fcγ receptor. Human activating Fcγ receptors include FcγRI, FcγRIIA, FcγRIIC and FcγRIIIB, whilst FcγRIIB is an inhibitory FcγR. Mouse activating Fcγ receptors include FcγRI, FcγRIII, and FcγRIV. Mouse FcγRIIB is an inhibitory FcγR. Both mice and human FcγRs display different affinities for different IgG Fc domains. Table 3 displays the binding affinities of various human FcγRs for different human IgG Fc domains and Table 4 displays the binding affinities of various mouse FcγRs for different mouse IgG Fc domains.

TABLE 3
Affinities of human FcγR for human IgG
Fc domains. Adapted from Castro-Dopico, 2019.

| | Activatory | | | | | Inhibitory |
|---|---|---|---|---|---|---|
| | FcγR | | | | | |
| | FcγRI | FcγRIIA | FcγRIIC | FcγRIIIA | FcγRIIIB | FcγRIIB |
| Variants | — | R/H131 | — | F/V158 | — | — |
| IgG1 | 6 × 10⁷ | 3/5 × 10⁶ | 1 × 10⁵ | 1/2 × 10⁵ | 2 × 10⁵ | 1 × 10⁵ |
| IgG2 | — | 1/4 × 10⁵ | 2 × 10⁴ | 3/7 × 10⁴ | — | 2 × 10⁴ |
| IgG3 | 6 × 10⁷ | 9 × 10⁵ | 2 × 10⁵ | 0.8/1 × 10⁷ | 1 × 10⁶ | 2 × 10⁵ |
| IgG4 | 3 × 10⁷ | 2 × 10⁵ | 2 × 10⁵ | 2 × 10⁵ | — | 2 × 10⁵ |

TABLE 4
Affinities of mouse FcγR for mouse IgG
Fc domains. Adapted from Castro-Dopico, 2019.

| | Activatory | | | Inhibitory |
|---|---|---|---|---|
| FcγR | FcγRI | FcγRIII | FcγRIV | FcγRIIB |
| IgG1 | — | 3 × 10⁵ | — | 3 × 10⁶ |
| IgG2a | 3 × 10⁷ | 7 × 10⁵ | 3 × 10⁷ | 4 × 10⁵ |
| IgG2b | 1 × 10⁵ | 6 × 10⁵ | 2 × 10⁷ | 2 × 10⁶ |
| IgG3 | (+) | — | — | — |

### Professional/Non-Professional Antigen Presenting Cells

Certain immune cells, such as dendritic cells and particular macrophage populations, are considered “professional” antigen presenting cells (professional APCs). While most cell types can perform antigen presentation on MHC class I molecules (specifically when the antigen has been synthesised intracellularly (endogenous antigen)), professional APCs can additionally process and present exogenous antigens on MHC class II and/or cross-present exogenous antigens on MHC class I molecules. Whether a professional APC engages in antigen presentation on MHC-II or MHC-I is also affected by their cell type and the nature of the antigen. Importantly, cDC1, a type of DC, are particularly adept at XP of cell-associated antigens, such as tumour antigens, due in part to their unique receptor expression pattern and have critical roles in anti-tumour immunity. By binding to the Fc domain of the bispecific binding agent of the invention, professional APCs that do not specialise in XP of cell-associated antigens (cDC2, monocyte-derived cells) and potentially non-professional cells expressing FcγR (neutrophils) can be enabled to cross-present necrotic cell-associated antigens on MHC-I for activation of CD8⁺ T cells in the context of necrotic cells.

### Pharmaceutical Compositions

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204 (s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. the vectors, cells and or chimeric receptors, used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

### Routes of Administration

Medicaments and pharmaceutical compositions according to aspects of the present invention may be formulated for administration by a number of routes, including but not limited to, parenteral, intravenous, intra-arterial, intramuscular, intratumoural, oral and nasal. The medicaments and compositions may be formulated in fluid or solid form. Fluid formulations may be formulated for administration by injection to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Combinations with Other Anticancer Treatments

As described herein, the medical methods, medical uses and pharmaceutical compositions of the invention may involve the bispecific binding agent in combination with another anticancer treatment. In some embodiments, the anticancer treatment is an additional immunotherapy.

Currently, the most common cancer immunotherapies are checkpoint inhibitors. The bispecific binding agent of the invention may be used in combination with a checkpoint inhibitor. Checkpoint inhibitors suitable for use in combination with the bispecific binding agent of the invention includes a checkpoint inhibitor that inhibits CTLA4, cytotoxic T-lymphocyte-associated antigen 4; e.g. anti-CTLA4; anti-LAG3, lymphocyte activation gene 3; anti-PD1, programmed cell death protein 1 (e.g., KEYTRUDA); PDL, anti-PD1 ligand; anti-TIM3, T cell membrane protein 3, anti-CD40L, anti-A2aR, adenosine A2a receptor; anti-B7RP1, B7-related protein 1; anti-BTLA, B and T lymphocyte attenuator; anti-GAL9, galectin 9; anti-HVEM, herpesvirus entry mediator; anti-ICOS, inducible T cell co-stimulator; anti-IL, interleukin; anti-KIR, killer cell immunoglobulin-like receptor; anti-LAG3, lymphocyte activation gene 3; anti-VISTA, V domain Ig Suppressor of T cell Activation; anti-B7-H3; anti-B7-H4; anti-TGFβ, transforming growth factor-β; anti-TIM3, T cell membrane protein 3; or anti-CD27.

Other immunotherapies, such as T cell therapy, can be used in conjunction with the bispecific binding agents disclosed herein. T cell therapies include administration of autologous or allogeneic T cells. In some embodiments, the bispecific binding agent is administered in combination with a CAR-T cell (a T cell that expresses a chimeric antigen receptor).

In some embodiments, the anticancer treatment is a cytotoxic chemotherapeutic, meaning that the bispecific binding agent of the invention may be used in combination with a cytotoxic chemotherapeutic. Combination with a cytotoxic chemotherapeutic has the advantage of leading to necrosis and exposure of F-actin on tumour cells, thus allowing the bispecific binding agent to bind to the tumour cells. Cytotoxic chemotherapeutic agents non-exclusively relates to alkylating agents, anti-metabolites, plant alkaloids, topoisomerase inhibitors, antineoplastics and arsenic trioxide, carmustine, fludarabine, IDA ara-C, myalotang, GO, mustargen, cyclophosphamide, gemcitabine, bendamustine, total body irradiation, cytarabine, etoposide, melphalan, pentostatin and radiation.

In some embodiments, the anticancer treatment is radiotherapy. In some embodiments, the anticancer treatment is surgery.

### Subject

The subject to be treated may be any animal or human. The subject is preferably mammalian, more preferably human. The subject may be a non-human mammal, but is more preferably human. The subject may be male or female. The subject may be a patient. Therapeutic uses may be in human or animals (veterinary use).

### Cancers

A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor. In some embodiments, the cancer is not a prostate cancer. Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

Some cancers cause solid tumours. Such solid tumours may be located in any tissue, for example the pancreas, lung, breast, uterus, stomach, kidney or testis. In contrast, cancers of the blood, such as leukaemias, may not cause solid tumours—and may be referred to as liquid tumours.

The cancer that is the subject of the treatments and medical uses of the present invention may be selected from the lists provided above.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Materials & Methods

### Mice

C57BL/6Jax, human FcγR-transgenic, Clec9a^{cre/cre}, sGsn^{-/-}, and OT-I/Rag1^{-/-} mice were bred at the Francis Crick Institute under specific-pathogen-free conditions. Female mice were used at 6-8 weeks of age for in vivo tumour experiments. All animal experiments were performed in accordance with national and institutional guidelines for animal care and were approved by the Francis Crick Institute Biological Resources Facility Strategic Oversight Committee (incorporating the Animal Welfare and Ethical Review Body) and by the Home Office, United Kingdom.

### Cell Lines

RPMI 1640 (Gibco) supplemented with glutamine, penicillin, streptomycin, 2-mercaptoethanol (all from Gibco) and 10% heat-inactivated foetal calf serum (R10 medium) was used for culture of RAW264.7, MCA205 fibrosarcoma, and BRAFV600E mutant 5555 melanoma cell lines. R10 was additionally supplemented with 10 mM HEPES, sodium pyruvate, and non-essential amino acids (all from Gibco) (termed R10+ medium) for culture of FLT3L-differentiated bone marrow-derived dendritic cells (FLT3L-cDCs), GM-CSF-derived cells (GMC), HoxB8-derived cDC2, and OT-I/Rag1^{-/-} T cells. LL171 bioassay cells were maintained in DMEM containing 10% FCS, and geneticin (Gibco).

For FLT3L-cDCs, bone marrow was extracted from hind legs of mice and subjected to red blood cell lysis (Thermo Fisher Scientific). Cells were then incubated for 9 days in R10+ medium containing 150 ng/ml FLT3L. On day 8, FTL3L cDC cultures were additionally primed with 200 ng/ml IFNα (R&D systems). cDC1 were enriched using biotinylated anti-mouse XCR-1 lgG (Biolegend, ZET clone), Anti-Biotin MicroBeads, and LS Columns (both Miltenyi) according to manufacturer's instructions. Purified cDC1 and non-cDC1 were then used for downstream applications. Alternatively, cDC2 were enriched on day 9 using PE-conjugated anti-mouse CLEC10A IgG (Biolegend, clone LOM-14) and Anti-PE MicroBeads (Miltenyi) according to manufacturer's instructions, followed by overnight priming with 200 ng/ml IFNα. GMC were generated from bone marrow cells cultured with 20 ng/ml GM-CSF for 7 days, as previously described (Helft, 2015), followed by overnight priming with 200 ng/ml IFNα. For HoxB8-derived cDC2, common dendritic cell progenitors (CDPs) were sorted from bone marrow and subjected to transduction as previously described (Kirkling, 2018). cDC2 were generated from HoxB8-CDPs using 75 ng/ml FLT3L for 7 days.

For pre-activated effector OT-I cultures, single cell suspensions were generated from spleens of OT-I/Rag1^{-/-} mice, and subjected to red blood cell lysis. Cells were incubated with R10+ medium supplemented with 100 U/ml IL-2 (Peprotech) and 0.1 nM SIINFEKL (generated at the Francis Crick Institute) for 3 days. On days 3 and 4, cells were split 1:2 and culture medium completely replaced with R10+ medium containing 100 U/ml IL-2. OT-I cultures were used for assays on day 5.

### DNGR-1-Fc Fusion Proteins

Mouse DNGR-1-mouse IgG2a Fc (6776-CL) and human DNGR-1-human IgG1 Fc (6049-CL) fusion proteins were purchased from R&D systems. Mouse DNGR-1-mouse IgG2a Fc and variant fusion proteins were also generated at the Francis Crick Institute. Briefly, WT and 2WA mouse DNGR-1 isoform 4 (long) ECD DNA sequences were amplified from existing pFB neo plasmids using Infusion-designed primers (Sigma). Amplified pFUSEN-mG2aFc plasmid (InvivoGen) was linearised using NheI and EcoRV restriction enzymes, gel purified, and subjected to Infusion reaction (Takara) with amplified 2WA or WT mouse DNGR-1 ECD DNA sequences according to manufacturer's instructions. Plasmids were transformed into Stellar competent cells and subjected to overnight selection on zeocin agar plates. Single colonies were cloned, sequenced, and used for downstream expression. For mouse DNGR-1-mouse IgG2a N297A Fc mutagenesis, sequence-verified WT DNGR-1-Fc was subjected to Quickchange Lightning Site-directed Mutagenesis Kit (Agilent Technologies) according to manufacturer's instructions, using designed primers spanning mutation site (Sigma). Plasmids were transformed into XL10 Gold bacteria (Agilent Technologies) and grown overnight on LB agar plates, single colonies grown in zeocin-containing LB broth, plasmid DNA extracted using QIAprep Spin Miniprep Kit (QIAGEN), and sequenced.

Amplified sequence-verified plasmids were transiently transfected into Expi293F cells and supernatants harvested over several days. Mouse DNGR-1-Fc fusion proteins were purified using protein A beads (Generon; M1300-5) following Pierce gentle Ag/Ab Binding and Elution Buffer Kit (Thermofisher, 21030) protocol. Proteins were then dialysed in endotoxin-free 25 mM Tris pH7.2, 150 mM NaCl (BupH Tris Buffered Saline Packs, Thermo Fisher Scientific). Samples were tested for endotoxin (Pierce Chromogenic Endotoxin Quant Kit-60 reactions, Thermo Fisher Scientific) and confirmed to be <0.05 EU/ml.

### Plate Stimulation

96-well high-affinity Nunc MaxiSorp plates (Thermo Fisher Scientific) were coated with 2 μg/ml or doubling dilutions of DNGR-1-Fc or molar equivalent mIgG1/mIgG2a (Biolegend) or Affimer-Fc in PBS overnight at room temperature. Plates were blocked with PBS containing 10% FCS for 1 h. RAW264.7 cells were plated at 1×10⁵ cells per well in triplicate in R10 medium for 24 h. Mouse TNF and CXCL2 were measured using R&D systems Duoset kits according to manufacturer's instructions.

### Generation of F-Actin/Myosin-II Complexes

FM complexes were prepared as previously described (Canton et al, 2021). Lyophilized nonbiotinylated G-actin and myosin II (Cytoskeleton) were reconstituted in sterile water at the final concentration of 10 mg/ml and stored at −80° C. Before use, the G-actin aliquots were diluted into G-actin buffer to final concentration 1 mg/ml and incubated for at least 30 min on ice. To generate biotinylated F-actin, nonbiotinylated G-actin was mixed in a 1:1 molar ratio with biotinylated G-actin (Cytoskeleton), which was always freshly reconstituted. Then, 20 μg of each G-actin preparation was mixed with F-actin buffer and incubated for 1 h at room temperature. To complex the biotinylated F-actin with myosin II, F-actin was mixed in a 1:1 molar ratio with myosin II and incubated for 1 h at room temperature.

### Preparing FM Beads and Necrotic Tumour Cells

For the coating of microbeads, OVA was biotinylated using the DSB-X biotinylation kit (Thermo Fisher Scientific). The concentration of biotinylated OVA was adjusted to 2 mg/ml. Streptavidin coated microbeads with a diameter of 2.0 μm (Polysciences) were used in nonfluorescent or yellow-green fluorescent form and were labeled with biotinylated OVA (1:1,000) for 1 h on ice. The OVA bead preparations were washed with 1% BSA in PBS for 3 min at 10,000×g. The OVA beads were then subjected to labelling with F-actin-myosin II. In vitro polymerized biotinylated F-actin-myosin II was added to OVA beads and incubated for at least 1 h on ice.

For necrotic cells, BRAFV600E mutant 5555 tumour cells were irradiated with 240 mJ/cm² UVC in PBS and left to die overnight in RO medium (lacking 10% FCS).

### Dead Cell Binding Assays

UV-irradiated 5555 necrotic cells were incubated with molar equivalent concentrations of mC9-hFc, mC9-mFc or Affimer-Fc for 1 h in PBS. Binding was detected using anti-mouse DNGR-1 or anti-mouse IgG antibodies conjugated to PE (1F6 clone, Biolegend) or A647, respectively, and assessed by flow cytometry.

### Phagocytosis Assays

Prior to irradiation, UV-5555 tumour cells were labelled with Cell Tracker-Deep Red (CT-DR) dye (Thermo Fisher Scientific; 1,1000 dilution) for 0.5-1 h at 37° C. CT-DR-labelled dead cells were added to RAW264.7 cells, FLT3L-cDC, or GM-CSF cells at indicated ratios and timepoints. Yellow-green microbeads (PolySciences) were FM-coated the day before analysis and applied to the cells at a 20/10:1 ratio. Following dead cell incubation, cells were surface stained for flow cytometry: CD11b (Biolegend, M1/70), XCR1 (Biolegend, ZET), CD172a (Biolegend, P84), I-A/I-E (Biolegend, M5/114.15.2), B220 (BD Biosciences, RA3-6B2), and CD11c (Biolegend, N418). DAPI or live-dead dye (Thermo Fisher Scientific) was used to exclude non-internalised dead cell material. Data were acquired on an LSR Fortessa flow cytometer (BD Biosciences) and analysed using FlowJo software.

### Cross-Presentation Assays

Antigen presenting cells (as specified) were plated at 5×10⁴ cells per well in U-bottomed 96-well plates. UV-5555 dead cells were soaked for 1 h in 10 mg/ml albumin from chicken egg white (OVA, Sigma) in RO and washed 3× in PBS. OVA-dead cells, FM-OVA beads, or SIINFEKL peptide were incubated with APCs at indicated ratios/concentrations for 4 h in the presence of DNGR-1-Fc fusion protein variants (1 μg/ml). 2:1 pre-activated OT-I effector cells were added for 24 h and T cell-derived IFNγ measured by ELISA.

For inhibitor studies, APC-T cell co-cultures were additionally treated with lactacystin (10 μM, Sigma) or leupeptin (250 μM, Sigma) for the duration of the experiment. For analysis of secreted gelsolin, co-cultures were performed using RO medium supplemented with 2.5% normal mouse serum or sGsn^{-/-} mouse serum. For Fcgr2b KO cultures, genetic deletion was performed as previously described (Freund, 2020) using Fcgr2b sgRNA or control sgRNA (IDT), and FcγRIIB protein reduction in FLT3L-cDC confirmed by flow cytometry on day 9 (anti-mouse CD32b IgG-PE, Thermo Fisher Scientific, AT130-2).

### DC Activation Assays

UV-5555 dead cells were soaked for 1 h with 62.5 μg/ml poly(I:C) and washed 3× in PBS. Dead cells were then added to cDC cultures, as described above, in the absence of T cells for 24 h. Cell-free supernatants were then added to interferon stimulating response element (ISRE)-luciferase-containing LL171 cells for 6 h. Luciferase activity was then assessed using the ONE-Glo luciferase assay system (Promega) according to manufacturer's instructions.

### In Vivo Tumour Experiments

MCA205 tumour cells were dissociated with trypsin (0.25%) and washed 3× in PBS. The final cell pellet was resuspended and diluted in endotoxin-free PBS (between 0.2×10⁶ to 0.5×10⁶ cells per 100 μl) and injected s.c. in the shaved right flank of each recipient C57BL/6Jax mouse. Tumour growth was monitored every 1 to 3 days, and the longest tumour diameter (I) and perpendicular width (w) were measured using digital Vernier callipers; tumour volume was calculated using the formula: length x width²/2 and expressed as mm³. Francis Crick Institute-generated 2WA or WT mC9-Fc was administered via intratumoural injection (50, 100 or 200 μg in 50 μl PBS) on day 7 and 11. For chemotherapy, mice were additionally injected with 2.5 mg/kg doxorubicin (Merck Life Science UK) or PBS on day 7. For radiotherapy, mice additionally received a 10 Gy localised dose of X-ray radiation at the tumour site using lead shields. Irradiation was performed on day 6 under general anaesthesia.

### Affimer-Fc Fusion Proteins

Affimer-Fc fusion proteins were generated by FairJourney Biologics. Affimers 6, 14, and 24 bind to F-actin and Control Affimer does not bind to F-actin (negative control). All Affimers used in the examples are conjugated to mouse IgG2a Fc (mFc), optionally via a linker, at their N-terminus.

sGSN Inhibition

For sGSN inhibition assays, recombinant mouse sGSN was generated by FairJourney Biologics. sGSN was incubated in PBS containing 100 μM CaCl2 with UV-treated 5555 dead cells for 1 h. Dead cells were then incubated with 10 nM Affimer-Fc for 30 min before detecting binding by staining with anti-mouse lgG A647.

### DNGR-1 Cross-Blocking Assay

UV-treated 5555 dead tumour cells were incubated with equimolar amounts of Fc-fusion proteins for 1 h in PBS. Blocking of DNGR-1 binding was the assessed by incubating dead cells with FLAG-tagged mC9 for 30 min at 1 μg/ml for 30 min. Binding of Fc-fusion proteins and Affimer-Fc was detected using anti-mouse IgG and anti-FLAG IgG antibodies.

### Intratumoural APC Analysis

Human: The frequency of pre-annotated DC subsets (cDC1, cDC2, DC3) was investigated in the pan-cancer myeloid cell atlas (Cheng et al, 2021) using online interface (panmyeloid.cancer-pku.cn).

Mouse: Intratumoural APC frequency was assessed by flow cytometry in MCA205 tumours on day 11 from WT C57BL/6 mice. Tumours and spleens were mechanically dissociated and digested with collagense IV (Worthington) and DNase I (Roche). Cells were surface stained with the following antibodies: XCR1 (Biolegend, ZET), CD172a (Biolegend, P84), I-A/I-E (Biolegend, M5/114.15.2), B220 (BD Biosciences, RA3-6B2), CD11c (Biolegend, N418), CD88 (20/70, Biolegend), FcγRI (X54-5/7.1, Biolegend), FcγRIIB (AT130-2, eBioscience), FcγRIII (S17014E, Biolegend), and FcγRIV (9E9, Biolegend). Dead cells were stained for 20 min at room temperature using LIVE/DEAD fixable blue dead cell staining kit (Thermo Fisher Scientific). Data were acquired on an LSR Fortessa flow cytometer (BD Biosciences) and analysed using FlowJo software.

### Confocal Microscopy

XCR1⁺ cDC1 or XCR1-depleted non-cDC1 were plated on anti-MHC-II IgG-coated coverslips and incubated with cell tracker Deep Red (CT-DR)-labelled UV-irradiated 5555 necrotic cells for 2 h in the presence/absence of mC9-Fc. Cells were stained with fluorescein-labelled wheat germ agglutinin (1:1000, 30 min at room temperature; Vector laboratories) and mounted using ProLong Diamond Antifade Mountant (Thermo Fischer Scientific). Images were acquired on a Zeiss LSM880 inverted confocal microscope using a 40× objective. The proportion of cDC internalising CT-DR-labelled material was quantified using ImageJ software.

### Nucleic Acid Sensing in cDC

Genetic deletion of Mavs was performed as previously described (Freund et al, 2020). Non-cDC1 were isolated from control sgRNA or Mavs sgRNA-treated bone marrow FLT3L-derived cDC cultures and incubated (overnight, 37° C.) with UV-irradiated 5555 necrotic cells soaked in poly(I:C) (1 h, 100 μg/mL) and extensively washed. IFNβ was detected in the supernatant using a commercial ELISA kit (R&D systems).

### Human FcγR Triggering Assay

NUNC MaxiSorp™ plates (Thermo Fisher) were coated overnight with human IgG1 (hIgG1) or mC9-hFc proteins. Plates were then blocked with 10% FCS in PBS. M-CSF-derived bone marrow macrophages (BMDM) from hFcγR-transgenic mice were added to the plate for 16 hours, and the supernatants assessed for TNF production using R&D Systems Duoset kits.

Cross-Presentation by hFcγR APC

For FLT3L-cDCs from human FcγR-transgenic mice, bone marrow was extracted from hind legs of mice and subjected to red blood cell lysis (Thermo Fisher Scientific). Cells were then incubated for 9 days in R10+ medium containing 150 ng/ml FLT3L. On day 8, FTL3L cDC cultures were additionally primed with 200 ng/ml IFNα (R&D systems). cDC1 were enriched using biotinylated anti-mouse XCR-1 lgG (Biolegend, ZET clone), Anti-Biotin MicroBeads, and LS Columns (both Miltenyi) according to manufacturer's instructions. GMC were generated from bone marrow cells cultured with 20 ng/ml GM-CSF for 7 days, followed by overnight priming with 200 ng/ml IFNα.

For pre-activated effector OT-I cultures, single cell suspensions were generated from spleens of OT-I/Rag1^{-/-} mice, and subjected to red blood cell lysis. Cells were incubated with R10+ medium supplemented with 100 U/ml IL-2 (Peprotech) and 0.1 nM SIINFEKL (generated at the Francis Crick Institute) for 3 days. On days 3 and 4, cells were split 1:2 and culture medium completely replaced with R10+ medium containing 100 U/ml IL-2. OT-I cultures were used for assays on day 5.

5-10×10⁴ cDC1, non-cDC1, and GMC were incubated with OVA-soaked UV-5555 dead cells (1:1 fixed ratio)+/−mC9-hFc and pre-activated OT-I CD8⁺ T cells in U-bottomed 96-well plates. T cell-derived IFNγ was measured after 24 h.

### Commercial Anti-Actin IgG2a

Mouse anti-actin IgG2a (clone AC-40) targeting an actin isoform-conserved C-terminal epitope was purchased from Abcam. AC-40 was incubated in serial dilutions with UV-irradiated 5555 necrotic cells for 30 min at room temperature, and binding detected by incubation with goat anti-mouse IgG2a AF488 for 30 min at 4° C. Binding was compared to murine Fc-C9. Data were acquired on an LSR Fortessa flow cytometer (BD Biosciences) and analysed using FlowJo software. Cross-presentation was performed and analysed as previously described, using IFNα-primed CLEC10A-purified cDC2 and a 1:100 dilution of AC-40.

### Example 1—Proof of Concept Experiment Showing Mouse DNGR-1-Fc Construct can Trigger Fc Receptors and Bind to F-Actin

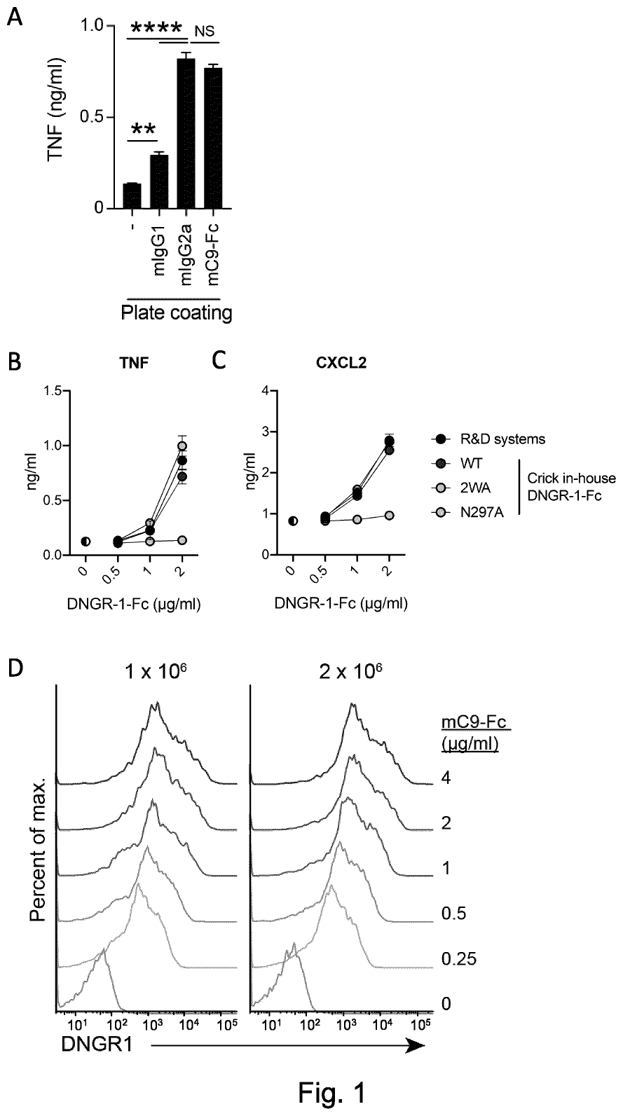

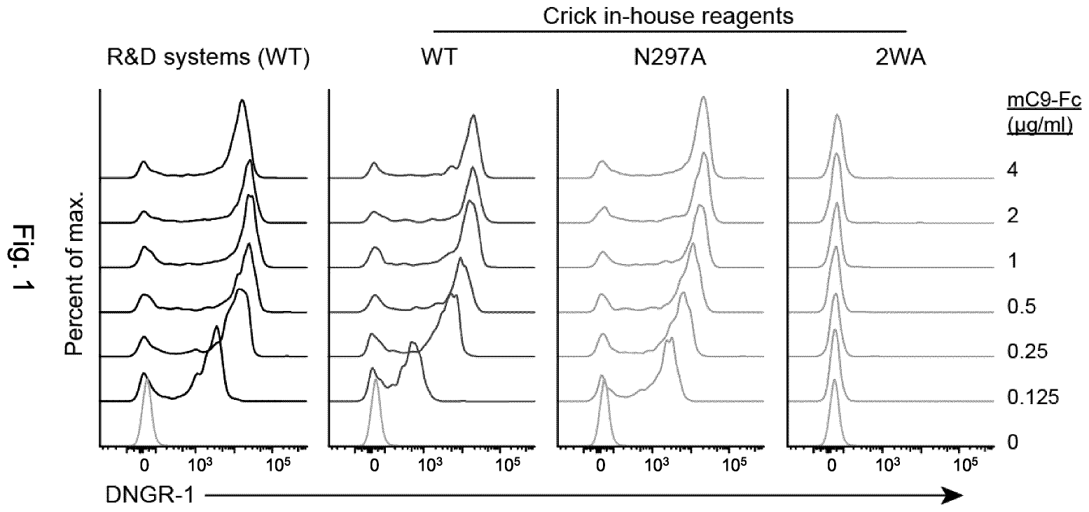

DNGR-1 is a type 2 membrane protein, meaning its extracellular domain is at the C-terminus of the molecule. Therefore, it was important to establish whether this orientation would affect the binding function of the construct. The inventors performed proof-of-concept experiments to show that a DNGR-1-Fc (C9-Fc) construct was capable of activating Fcγ receptors and of binding to F-actin. NUNC MaxiSorp™ plates (Thermo Fisher) were coated with mouse IgG1 (mIgG1), mIgG2a, or a mouse DNGR-1-Fc construct (mC9-Fc) obtained from R&D Systems (#6776-CL). RAW264.7 macrophages were added to the plate for 16 hours, and then the supernatants were assessed for TNF production. TNF is a cytokine produced by macrophages and is a standard readout of Fcγ receptor activation. The RAW264.7 macrophage cell line was chosen because they naturally express high levels of Fc receptors. The results displayed in FIG. 1A show functional engagement of the Fcγ receptor using the mC9-Fc construct, at a much higher level than murine IgG1.

To investigate this further, the inventors constructed an in-house WT murine DNGR-1-mIgG2a Fc construct (SEQ ID NO: 13) as well as 2 mutant murine DNGR-1-mIgG2a Fc constructs, using the pFUSEN plasmid from Invivogen. The 2WA mutant (SEQ ID NO: 15) contains 2 mutations in the CTLD DNGR-1 domain, and the N297A mutant (SEQ ID NO: 14) contains a mutation in the CH2 domain of the Fc domain of mIgG2a. The N297A mutant showed impaired triggering of Fcγ receptors, as shown in both a TNF assay (FIG. 1B) and a CXCL2 assay (FIG. 1C).

To investigate the ability of an mC9-Fc construct to bind to F-actin, F-actin/myosin-II (FM) beads were incubated with mC9-Fc for 1 hour. Binding was then detected using anti-DNGR-1 lgG PE. The mC9-Fc was able to bind to the beads (FIG. 1D). To confirm that the mC9-Fc constructs were able to bind to F-actin on dead cells, UV-treated 5555 dead cells were incubated with mC9-Fc for 1 hour and binding was detected again using anti-DNGR-1 IgG PE (FIG. 1E). Both the R&D and in-house WT constructs were capable of binding the necrotic cells, whereas the 2WA mutant showed a lack of binding, showing that binding specifically to F-actin was required.

These preliminary experiments provide confirmation that the mouse DNGR-1-Fc constructs are physically able to bind Fc receptors and trigger Fc receptor cross-linking and cell activation in RAW macrophages and that the construct can bind F-actin on both FM beads and on UV-irradiated tumour cells.

### Example 2—Mouse DNGR-1-Fc Construct can Induce Phagocytosis by Various Myeloid Cells

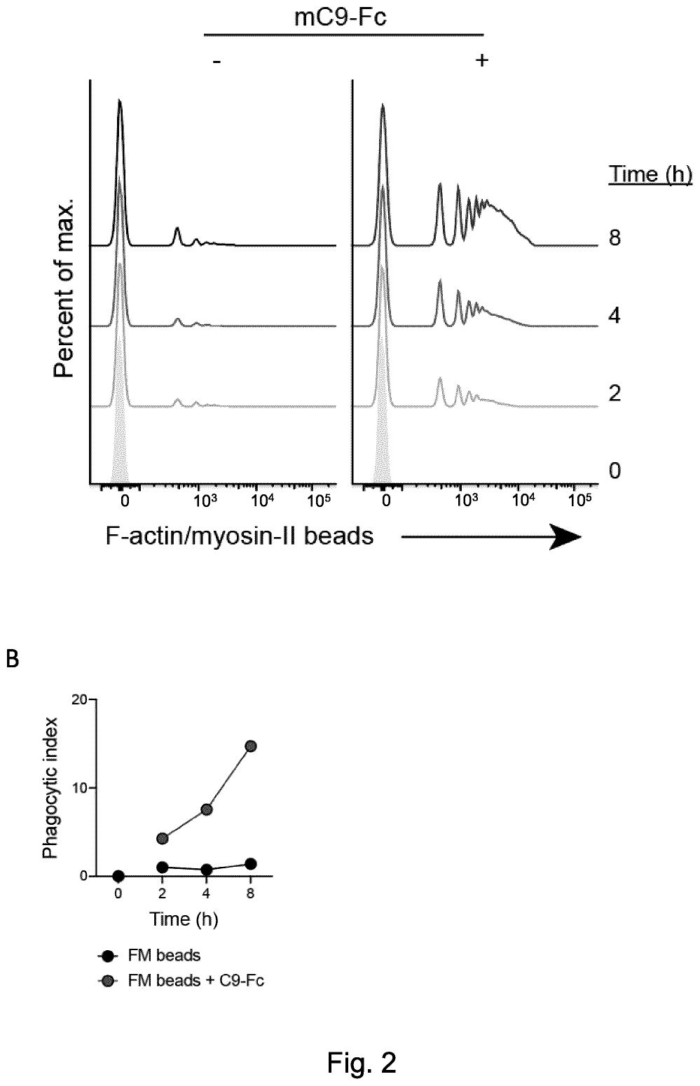

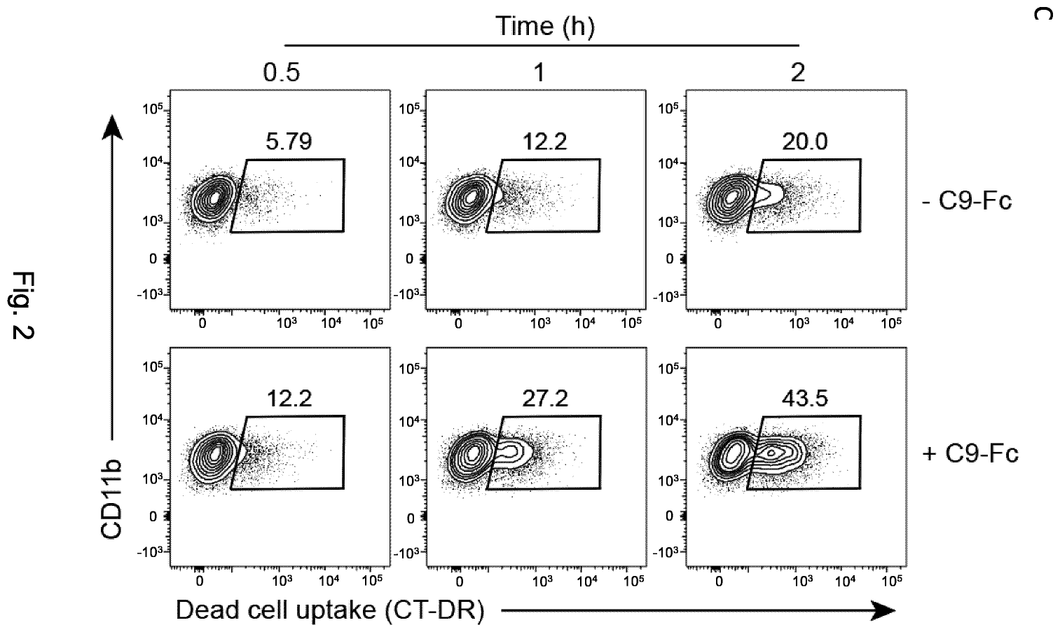

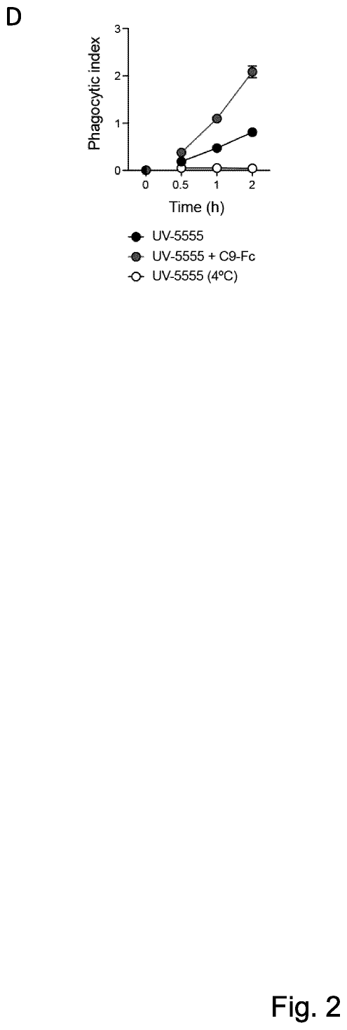

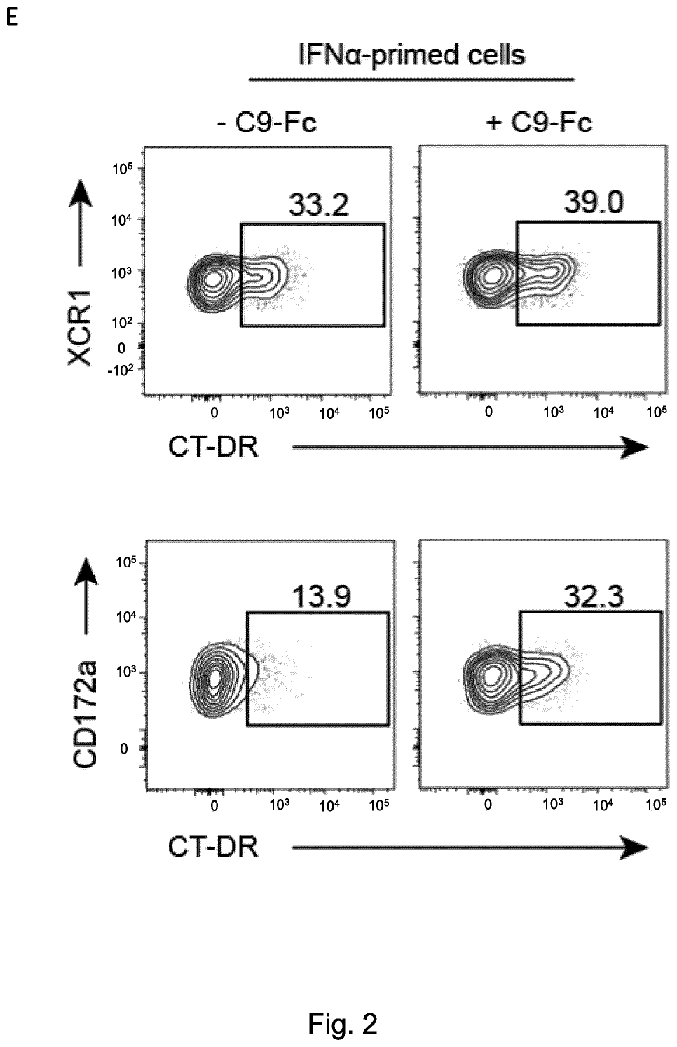

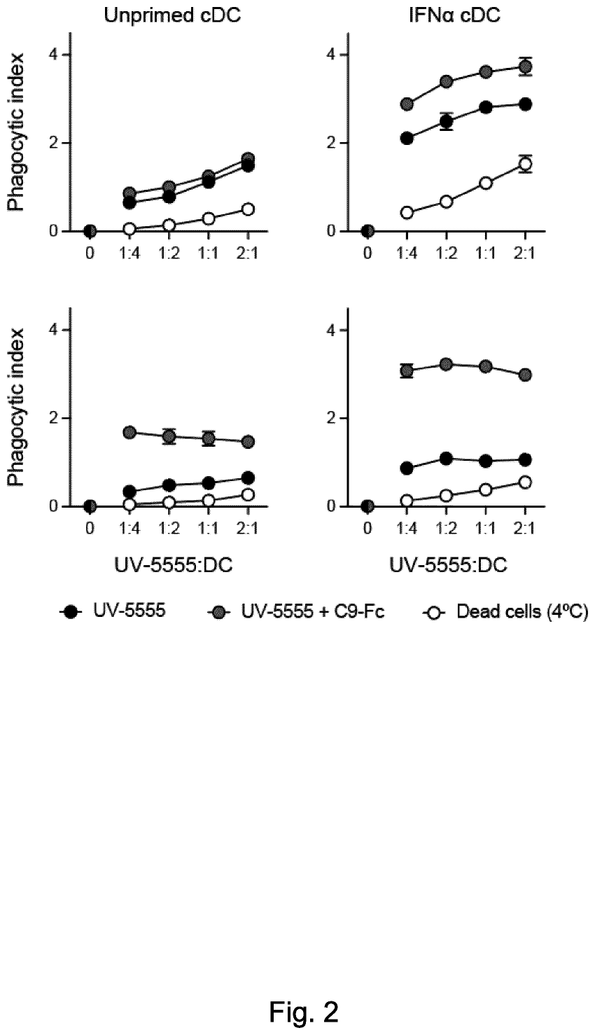

It is essential the bispecific binding agent can mediate phagocytosis because antigen processing and loading onto MHC class I molecules for XP occurs inside the cell. Therefore, the dead cell and its associated antigens must be internalised to access the processing machinery. The ability of the R&D mouse DNGR-1-Fc construct to induce phagocytosis was assessed in various myeloid cells. Phagocytosis of fluorescent FM-beads or of CT-DR-labelled UV-treated 5555 dead cells by RAW264.7 macrophages was measured by incubating the macrophages with FM-beads or with UV-treated 5555 dead cells and mC9-Fc for various timepoints, with uptake tracked by flow cytometry. mC9-Fc was shown to boost the uptake of FM-beads (FIGS. 2A and 2B) and of UV-treated 5555 dead cells (FIGS. 2C and 2D) by RAW264.7 macrophages.

Phagocytosis of CT-DR-labelled UV-treated 5555 dead cells by primary FLT3L-DC (cDC1 and cDC2) was measured as above by incubating the dendritic cells with UV-treated 5555 dead cells and mC9-Fc for 2 hours. mC9-Fc was shown to boost the uptake of UV-treated 5555 dead cells by both cDC1 and cDC2 cells (FIGS. 2E and 2F).

Phagocytosis of UV-treated 5555 dead cells by GM-CSF-derived cells (GMC) was measured in the same way as FLT3L-DC. mC9-Fc was shown to boost the uptake of UV-treated 5555 dead cells by the GMC (FIG. 2G).

The inventors have shown that the commercially available mC9-Fc can trigger phagocytosis by RAW macrophages, HoxB8-derived cDC2 line, GM-CSF cells and IFNα-primed primary cDC cells.

### Example 3—Mouse DNGR-1-Fc Induces Cross-Presentation

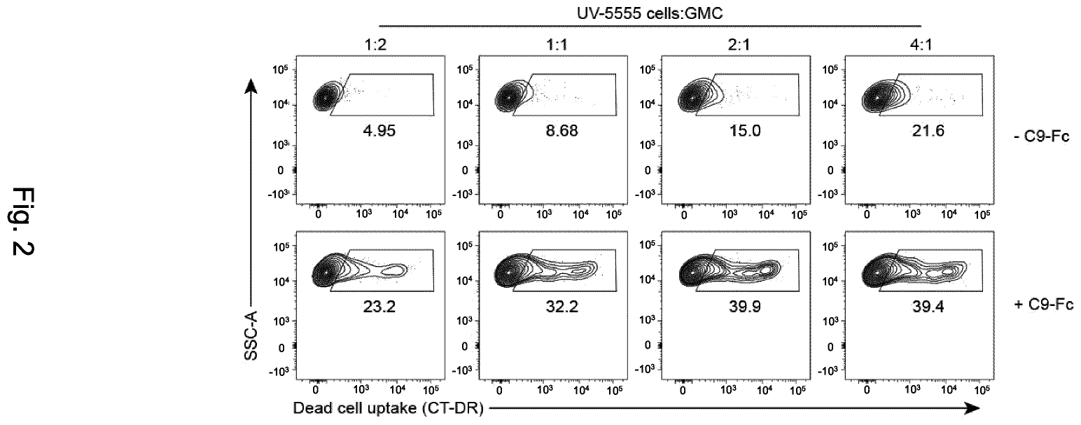

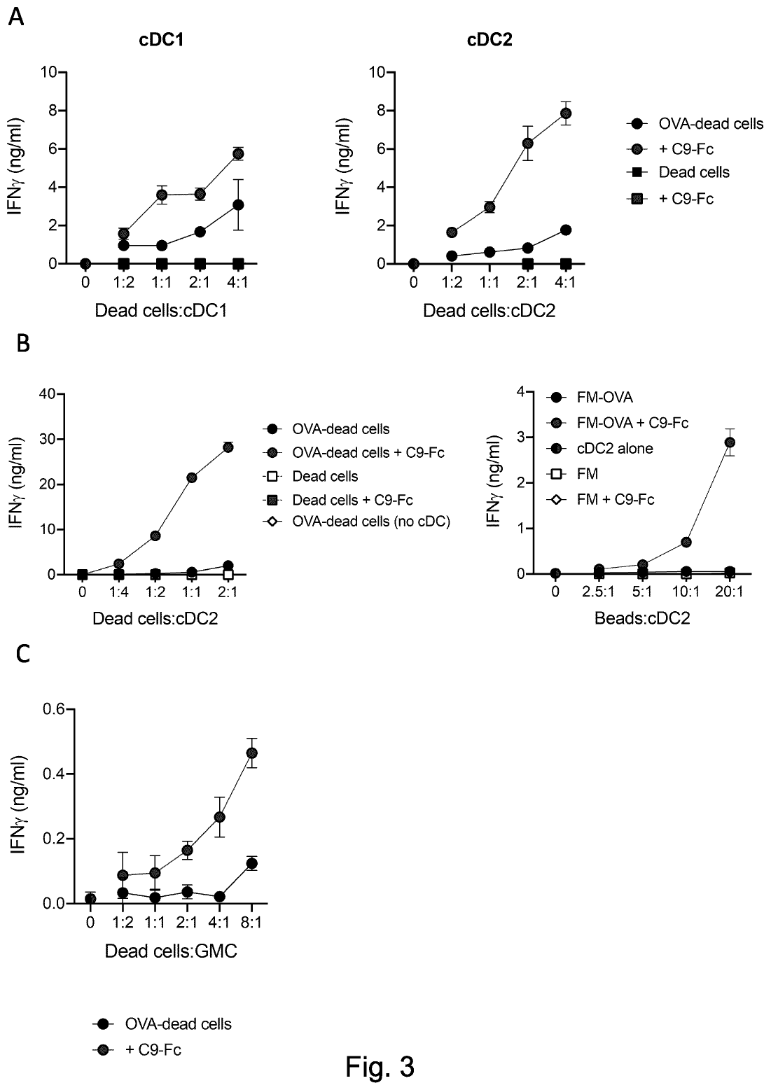

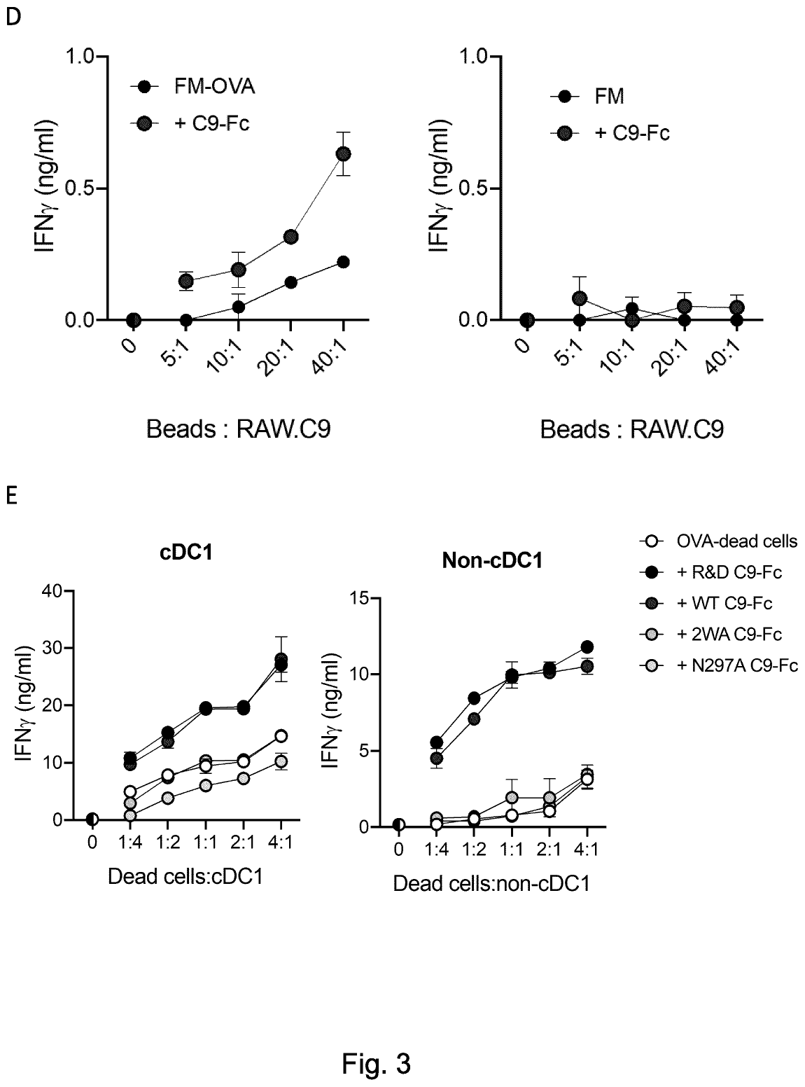

Primary FLT3L-DC subsets were isolated to 90% purity using either XCR1 enrichment (to isolate cDC1 cells) or CLEC10A enrichment (to isolate cDC2 cells), primed with IFNα, and incubated with OVA-soaked UV-5555 dead cells+/−R&D mC9-Fc and OT-I CD8⁺ T cells. T cell-derived IFNγ was measured after 24 h, with the R&D mC9-Fc shown to boost T cell activation in both DC subsets. This effect was most pronounced for cDC2, which lack the ability to cross-present dead cell antigens. Myeloid-T cell co-cultures were also performed with other FcγR-expressing myeloid cells and OVA-dead cells or OVA-FM-beads, with the R&D mC9-Fc also being shown to boost T cell activation by the HoxB8 cDC2 cell line (FIG. 3B), GM-CSF-derived cells (FIG. 3C) and RAW264.7.Clec9a.Kb cells (FIG. 3D).

After establishing the commercially available construct was capable of XP, the experiment was repeated using the in-house WT and mutant constructs with FLT3L-DC cultures. The in-house WT mC9-Fc construct boosted XP to the same extent as the R&D construct (FIG. 3E). Therefore, the longer linker used in the in-house construct does not impair the ability to trigger XP. The ability to boost XP was lost in the 2WA and N297A mutants, incapable of efficiently binding to F-actin or FcγR, respectively.

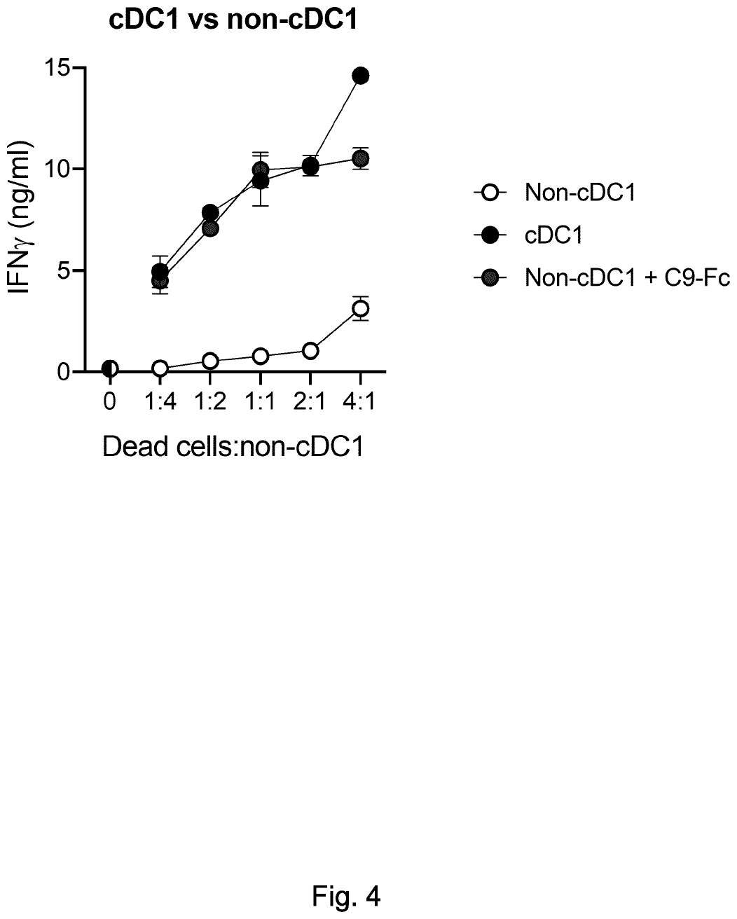

An advantage of the invention is the increase in the range of cells that can perform XP of dead cell antigens. This is highlighted in FIG. 4, which demonstrates that in-house mC9-Fc is able to endow non-cDC1 cells (which includes FcγR-expressing cDC2 and a minor population of macrophages, as well as plasmacytoid DC lacking FcγR) with an equal ability to XP dead cell antigens as cDC1 cells.

### Example 4—Investigating the Mechanism of Action of mC9-Fc

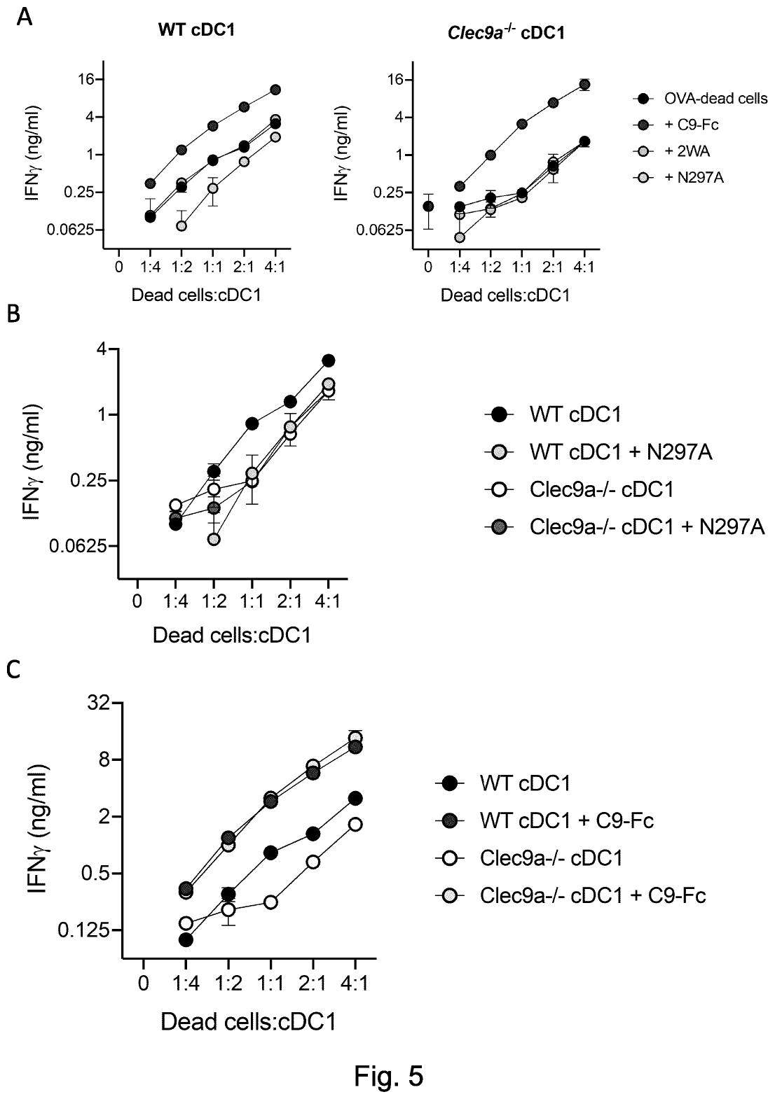

After establishing the mC9-Fc constructs were functional, the inventors sought to establish the mechanism behind the activity. With this aim, they first set out to investigate whether the mC9-Fc relied on endogenous DNGR-1 in FLT3L-cDC1 cells. Experiments were performed using the in-house constructs (WT, 2WA, and N297A mutants) in both WT and Clec9a/cDC1 cells and OVA-soaked UV-treated 5555 dead cells. As shown in FIG. 5A, in WT cDC1 cells, the WT construct was able to boost

XP, whilst the N297A mutant partially inhibited XP. In the DNGR-1 KO cells, the WT construct was still able to boost XP, whilst the N297A mutant did not inhibit XP. The ability of the mC9-Fc constructs to perform XP in the DNGR-1 KO cells indicates that the mC9-Fc does not rely on endogenous DNGR-1 to function.

The inventors next investigated whether the mC9-Fc construct interfered with endogenous DNGR-1 on cDC1 cells binding to F-actin. They measured XP by WT cDC1 cells incubated with the N297A mutant. This mutant is unable to efficiently bind to Fcγ receptor and so cannot trigger XP. This mutant showed the same level of XP inhibition as that seen in the DNGR-1 KO cells incubated with or without the N297A mutant construct (FIG. 5B). Because the N297A mutant construct is unable to trigger XP itself, the reduction in XP seen in the WT cDC1 cells shows that the DNGR-1-Fc construct inhibits endogenous DNGR-1 binding to F-actin, and thus prevents downstream XP.

To establish whether endogenous DNGR-1 plays a role in the mechanism of action of mC9-Fc, the inventors compared the XP ability of WT cDC1 cells incubated with the in-house mC9-Fc construct to the XP ability of DNGR-1 KO cDC1 cells incubated with the in-house mC9-Fc construct. A similar level of XP was obtained that was independent of the presence of endogenous DNGR-1 (FIG. 5C). Thus, mC9-Fc does not depend on endogenous DNGR-1 for XP.

### Example 5—The C9-Fc Construct Mediates Cross-Presentation Via the Cytosolic Pathway

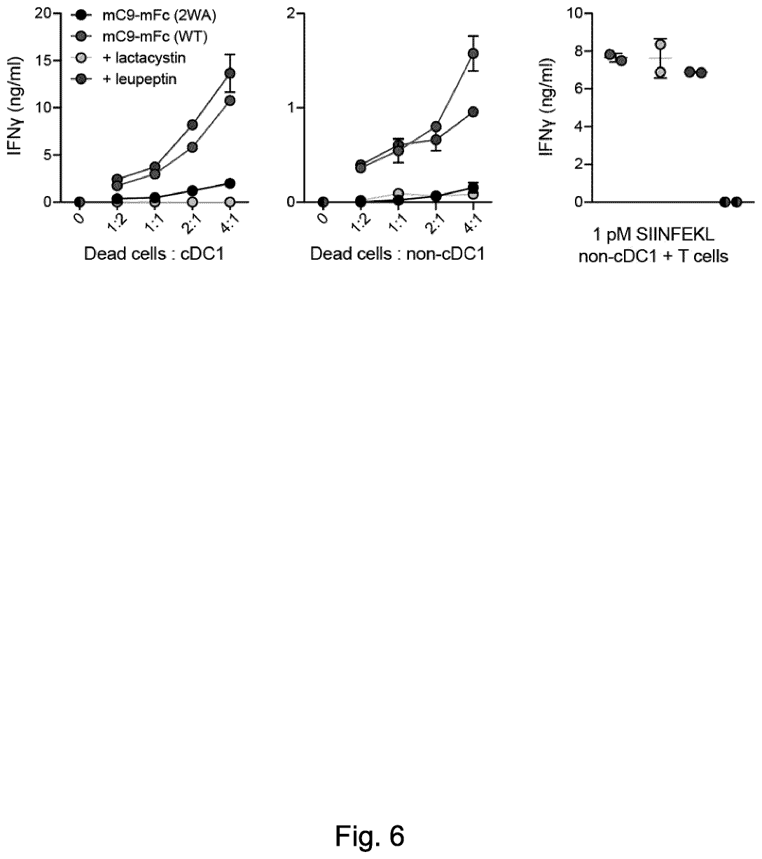

The inventors' previously filed PCT application PCT/EP2021/071399 demonstrated for the first time that DNGR-1 engages the cytosolic, not the vacuolar, XP pathway in cDC1 cells. Here, the inventors sought to discover which pathway the mC9-Fc construct triggers. With this aim, the effect of lactacystin (a proteasome inhibitor) and leupeptin (a protease inhibitor) on XP of OVA-soaked UV-treated 5555 dead cells by cDC1+mC9-Fc construct (in-house WT and 2WA mutant) was measured. The results show that XP of dead cell-OVA to OT-I by mC9-Fc was inhibited by lactacystin (FIG. 6), demonstrating a requirement on the cytosolic proteasome. Thus, mC9-Fc XP proceeds via the cytosolic pathway.

### Example 6—sGSN Attenuates mC9-Fc Dead Cell XP by Primary DC Cells

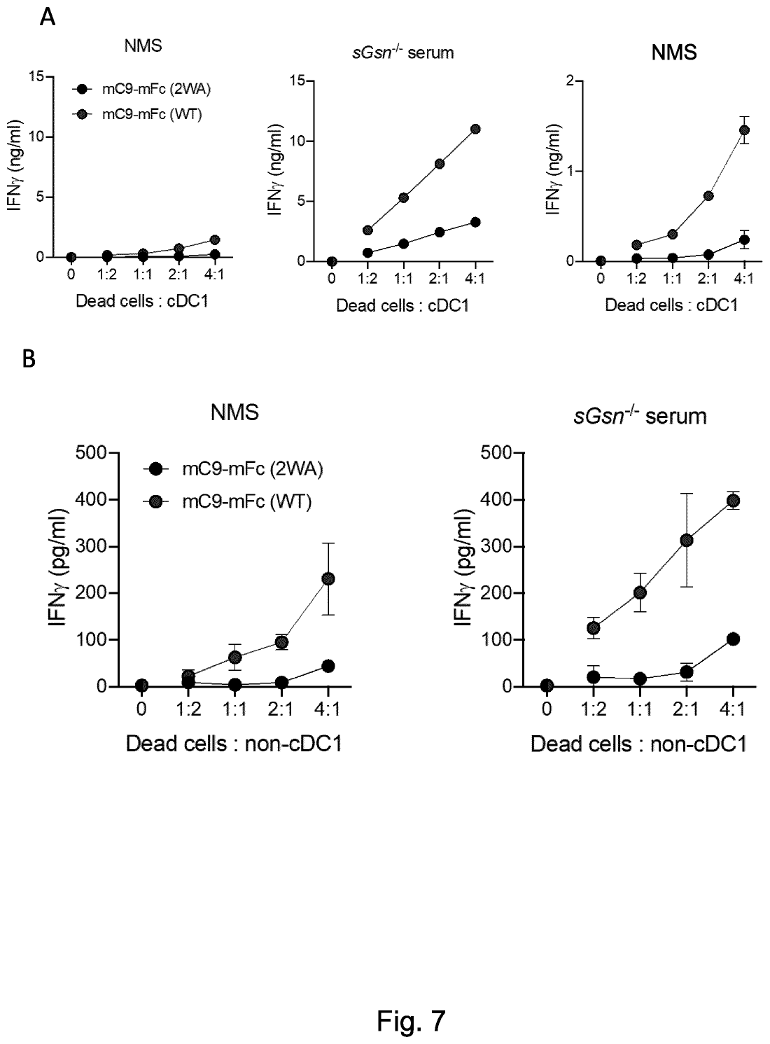

The effect of sGSN on the ability of the in-house mC9-Fc was investigated. sGSN binds to F-actin and severs the filaments for subsequent depolymerisation. sGSN therefore prevents F-actin binding to DNGR-1. Primary FLT3L-DC were primed with IFNα for 24 hours and split into cells enriched for XCR1 (to isolate cDC1) or XCR1-negative (non-cDC1). These were then incubated with OVA-soaked UV-treated 5555 dead cells and either normal mouse serum or sGSN^{-/-} serum and either WT in-house mC9-Fc or the 2WA mutant in the presence of OT-I T cells. The level of XP was assessed by measuring T cell-derived IFNγ production after 24 h. sGSN was shown to attenuate mC9-Fc dead cell XP in both cDC1 (FIG. 7A) and non-cDC1 (activity derived from cDC2) (FIG. 7B). The presence of serum containing sGSN significantly impaired XP by cDC1 to OT-I CD8⁺ T cells in vitro in the presence and absence of mC9-Fc (FIG. 7A). In contrast, XP by non-cDC1 was only impaired in the presence of the WT mC9-Fc (FIG. 7B).

Therefore, mC9-Fc XP is inhibited by sGSN in IFNα-primed cDC1 and cDC2.

### Example 7—mC9-Fc XP is not Affected by Inhibitory Fcγ Receptor IIB Expression on IFN-Primed cDC1/cDC2

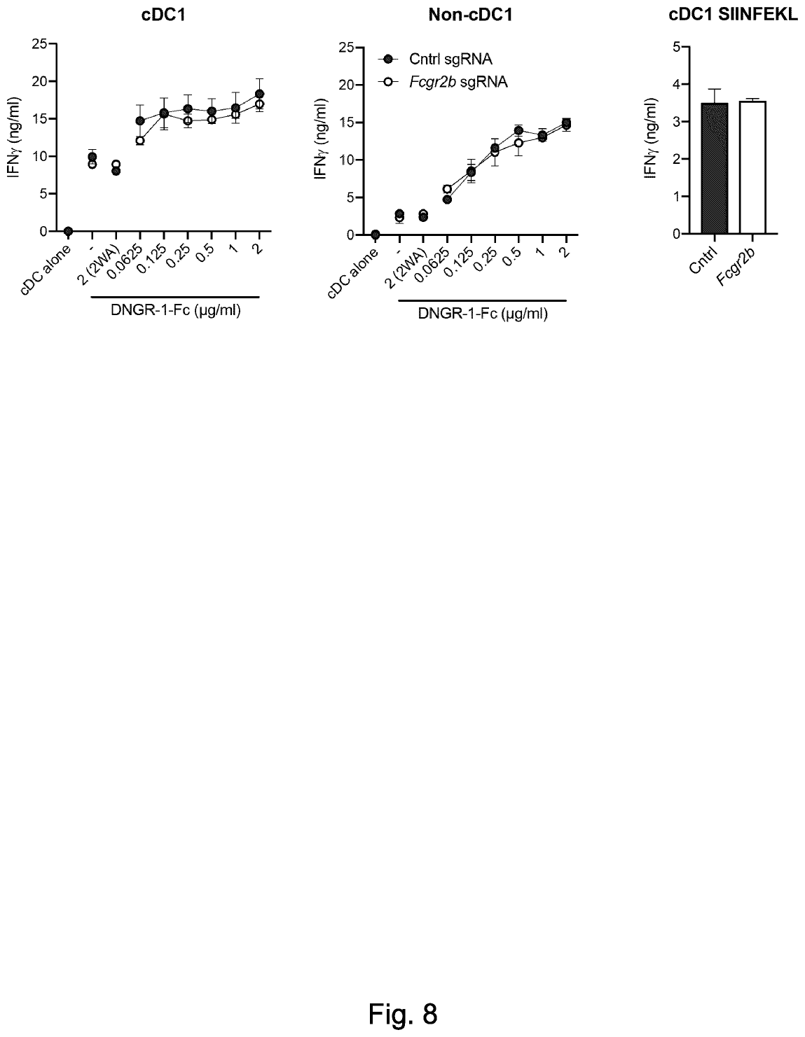

To understand the effect of the inhibitory Fcγ receptor FcγRIIB on XP of dead cells by the in-house mC9-Fc reagents, Fcgr2b KO hematopoietic bone marrow cells were generated using CRISPR-Cas9. These were then differentiated into FLT3L-DC. Along with control FLT3L-DC, the KO cells were primed with IFNα for 24 hours and split into cells enriched for XCR1 (to generate cDC1) or XCR1-negative (non-cDC1). These were then incubated with OVA-soaked UV-treated 5555 dead cells and either WT in-house mC9-Fc or the 2WA mutant. The Fcgr2b KO was shown to have no effect on the ability of the WT mC9-Fc construct to perform XP of dead cells (FIGS. 8A and 8B). Therefore, the expression of the inhibitory FcγRIIB receptor does not affect XP by mC9-Fc.

### Example 8—Myeloid Cell Activation with DAMPs

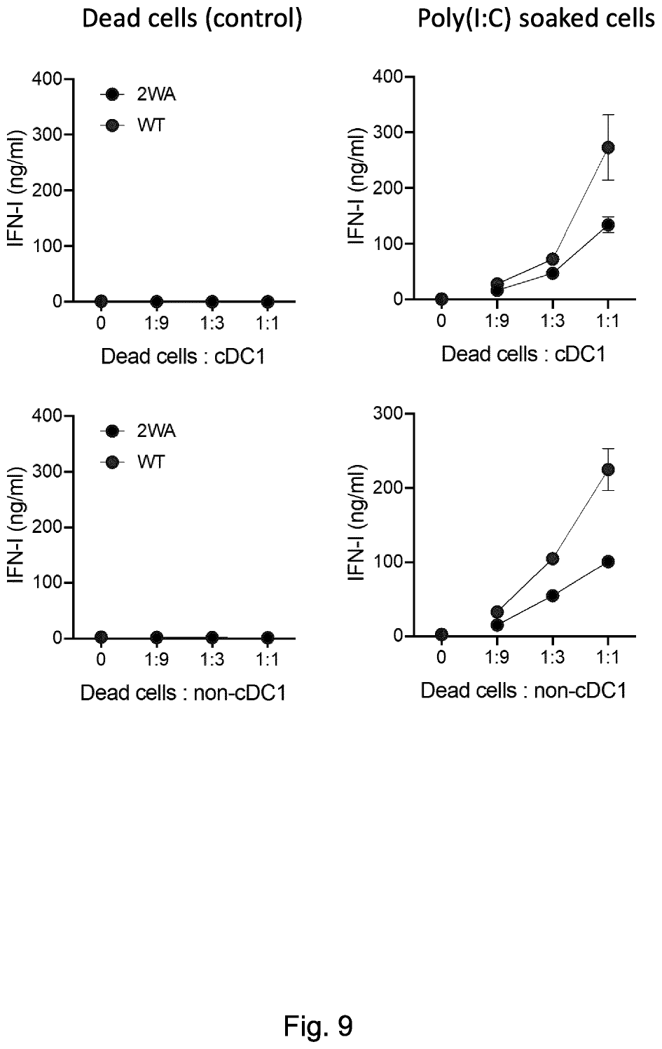

The ability of the in-house mC9-Fc construct to enhance the delivery of PAMPs and DAMPs to cDC was investigated. FLT3L-DC were primed with IFNα for 24 hours and split into cells enriched for XCR1 (to generate cDC1) or XCR1-negative (non-cDC1). These were then incubated with UV-treated 5555 dead cells+/−soaking in poly(I:C) and either WT in-house mC9-Fc or the 2WA mutant. Supernatants from cDC treated with control or poly(I:C)-soaked dead cells+/−mC9-Fc were added to LL171 bioassay cells that contain an interferon stimulating response element (ISRE)-luciferase construct. Luciferase activity was measured after 6 h. No T cells were present in the cDC cultures. WT mC9-Fc led to a boost in type I IFN (IFNα/B) production, particularly non-cDC1 (cDC2) (FIG. 9).

### Example 9—In Vivo Anti-Tumour Response with Mouse DNGR-1-Fc Construct

To assess the ability of the mC9-Fc construct in an in vivo disease setting, the in-house mC9-Fc construct (WT and 2WA mutant) was administered intratumourally to WT C57BL/6 mice with MCA205 tumours, in combination with doxorubicin to enhance immunogenic cell death within the tumour. 1 dose of doxorubicin was administered, and 2 doses of mC9-Fc were administered.

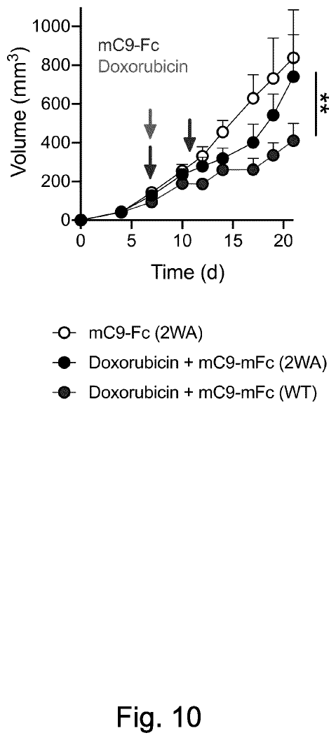

WT mC9-Fc delayed MCA205 tumour growth when administered with doxorubicin intratumourally (FIG. 10).

### Example 10—Preliminary Human DNGR-1-Fc Construct Experiments

Having established the mouse DNGR-1-Fc construct could enhance XP, the inventors then sought to establish whether a human DNGR-1-human Fc (hC9-Fc) construct was also capable of enhancing XP. For these preliminary experiments, the inventors used the commercially available recombinant Human CLEC9a Fc Chimera Protein (R&D Systems, 6049-CL). The DNGR-1 CTLD of the human construct was able to bind to UV-treated mouse 5555 dead cells (FIG. 11A). However, the human construct was not able to trigger mouse Fcγ receptors on RAW264.7 cells (FIG. 11B), nor augment XP in WT mouse FLT3L cDC cells (FIG. 11C).

This finding was surprising because it would be expected that the human construct would be able to bind and engage Fcγ receptors, especially in light of the experiments performed in mice described in the previous Examples.

Without wishing to be bound by any particular theory, this led the inventors to the hypothesis that the human construct may not efficiently cross-link Fcγ receptors due to the shorter neck region in human DNGR-1 limiting the flexibility of the construct, particularly due to the inverted topology of the Fc domain within the fusion protein. The inventors further hypothesise that this lack of functionality in the human construct may be rescued by the insertion of a longer linker between the Fc receptor-binding moiety and the CTLD of human DNGR-1. This hypothesis forms the basis of future planned experiments.

### Example 11—Affimer-Fc Binding Properties

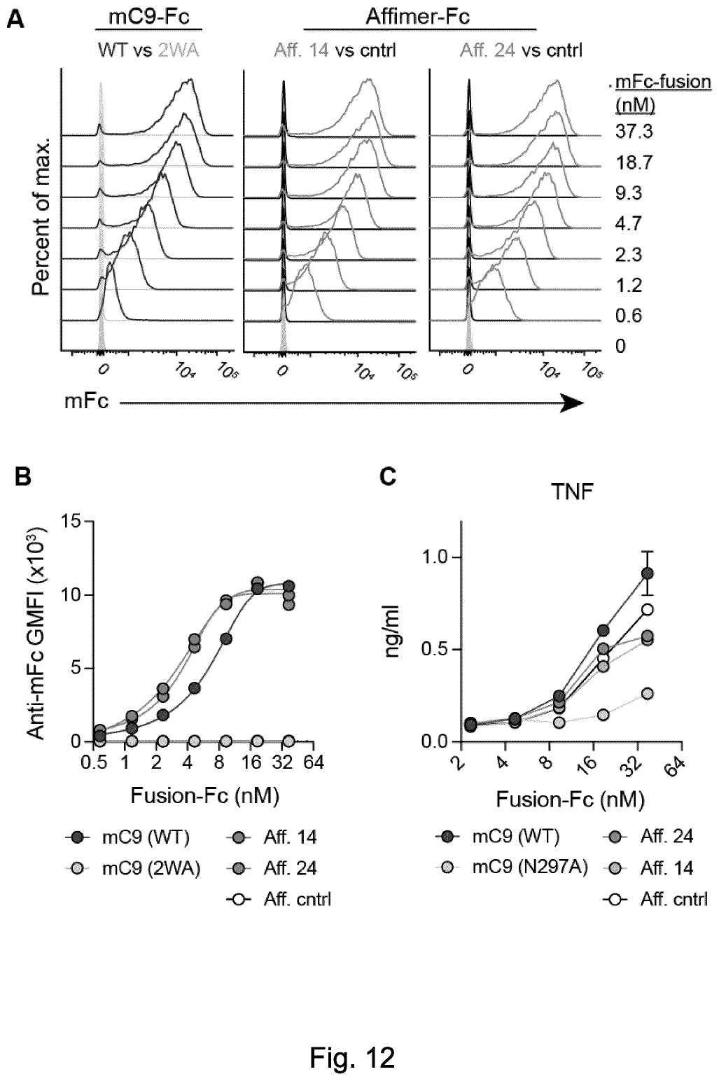

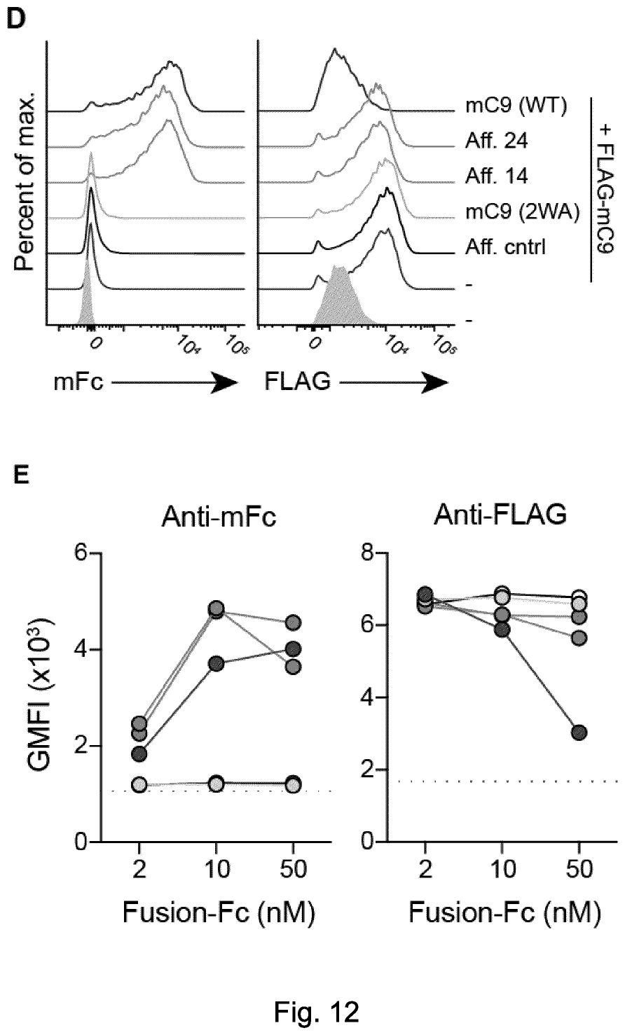

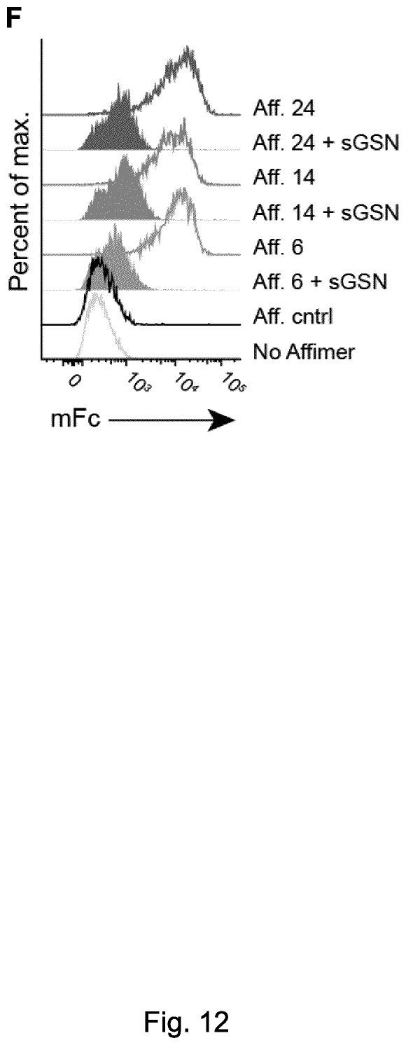

After establishing that bispecific binding agents that use DNGR-1 as the F-actin binding moiety can couple necrotic cell sensing to various downstream cellular responses (such as XP), the inventors sought to confirm that this coupling could be achieved using a bispecific binding agent with an alternative F-actin binding moiety. To this end, 3 different affimers specific for F-actin were conjugated to mouse IgG2a Fc (mFc) at their N-terminus. The affimer-Fc constructs used are termed Affimer 6, Affimer 14 and Affimer 24. To investigate the ability of the affimer-Fc constructs to bind to F-actin, the constructs were incubated with UV-treated 555 dead tumour cells for 1 hour. A control affimer that does not bind F-actin was included as a negative control. FIG. 12A shows that Affimers 14 and 24 were able to bind to F-actin. Binding was then detected using anti-mouse IgG-A647 (GMFI) (FIG. 12B).

To investigate whether the affimer-Fc constructs were able to trigger Fcγ receptors, NUNC MaxiSorp™ plates (Thermo Fisher) were coated overnight with affimer-Fc constructs. RAW264.7 macrophages were added to the plate for 16 hours, and the supernatants assessed for TNF production. Affimer-Fc constructs were able to trigger Fcγ receptors, as shown by measuring TNF by ELISA (FIG. 12C).

The cross-blocking effect of affimer-Fc on DNGR-1 (mC9) binding to F-actin was assessed by incubating UV-5555 dead cells with Fc-fusion proteins for 1h, followed by incubation with 1 μg/ml mC9-FLAG for 30 min. mC9-Fc was used as a positive control for binding inhibition. Binding of fusion proteins and mC9-FLAG was detected using anti-mouse IgG and anti-FLAG IgG, respectively. The right-hand panels of FIGS. 12D and 12E show that the affimer-Fc constructs did not block binding of DNGR-1 to the UV-5555 dead cells. The left-hand panels of FIGS. 12D and 12E show that all Fc-fusion proteins were capable of binding dead cells.

sGSN inhibition of affimer-Fc binding to dead cells was investigated by incubating UV-5555 dead cells with 10 μg/ml sGSN for 1 h at 4° C., followed by incubation with 10 nM Affimer-Fc. sGSN inhibited the binding of all 3 affimer-Fc constructs to dead cells (FIG. 12F).

These preliminary experiments provide confirmation that constructs which employ an affimer as the F-actin binding moiety are physically able to both bind Fc receptors and to trigger Fc receptor cross-linking. The affimer-Fc constructs do not prevent DNGR-1 binding to F-actin on dead cells. The affimer-Fc binding to F-actin is sensitive to inhibition by sGSN.

### Example 12—cDC and FcR Expression in Tumours

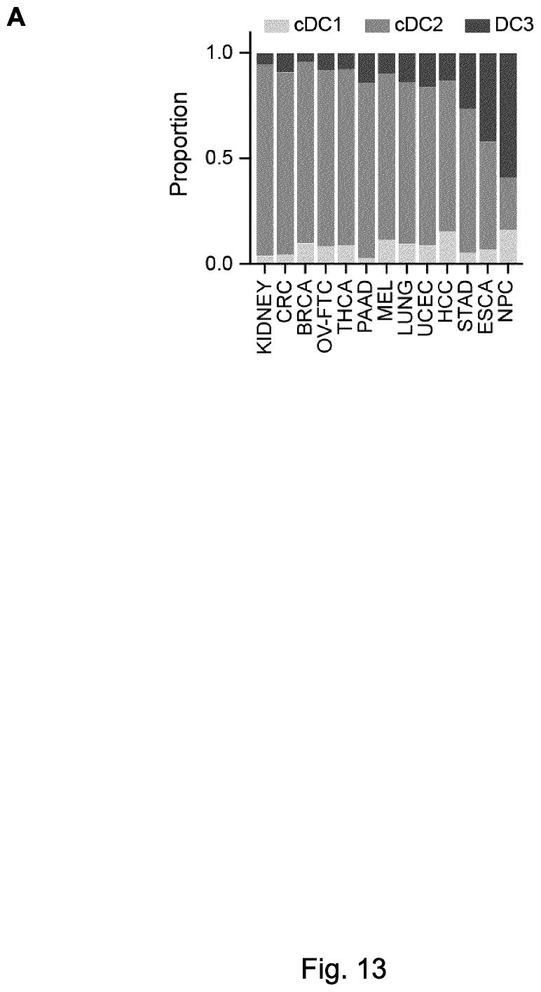

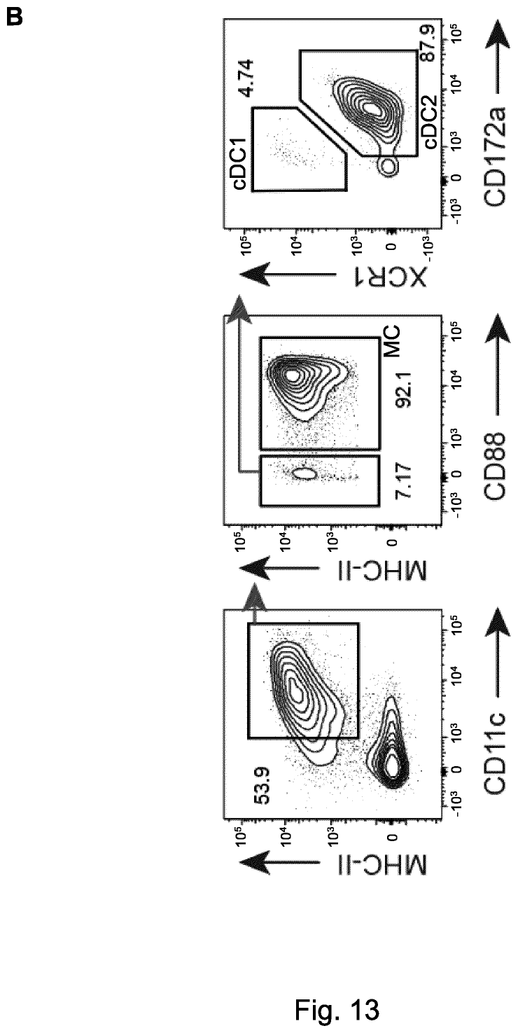

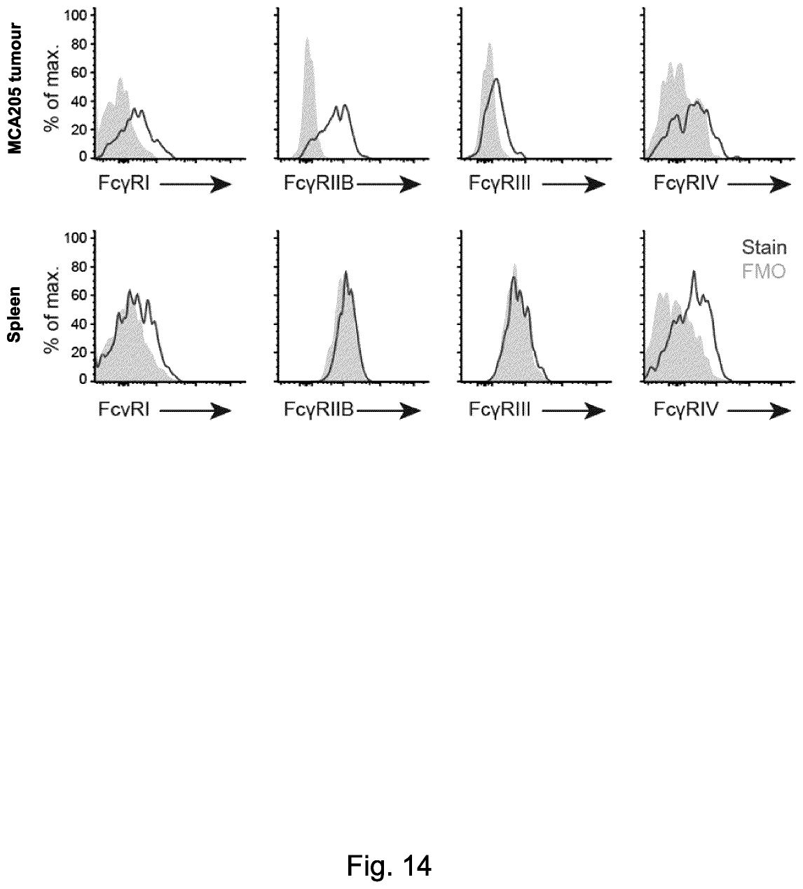

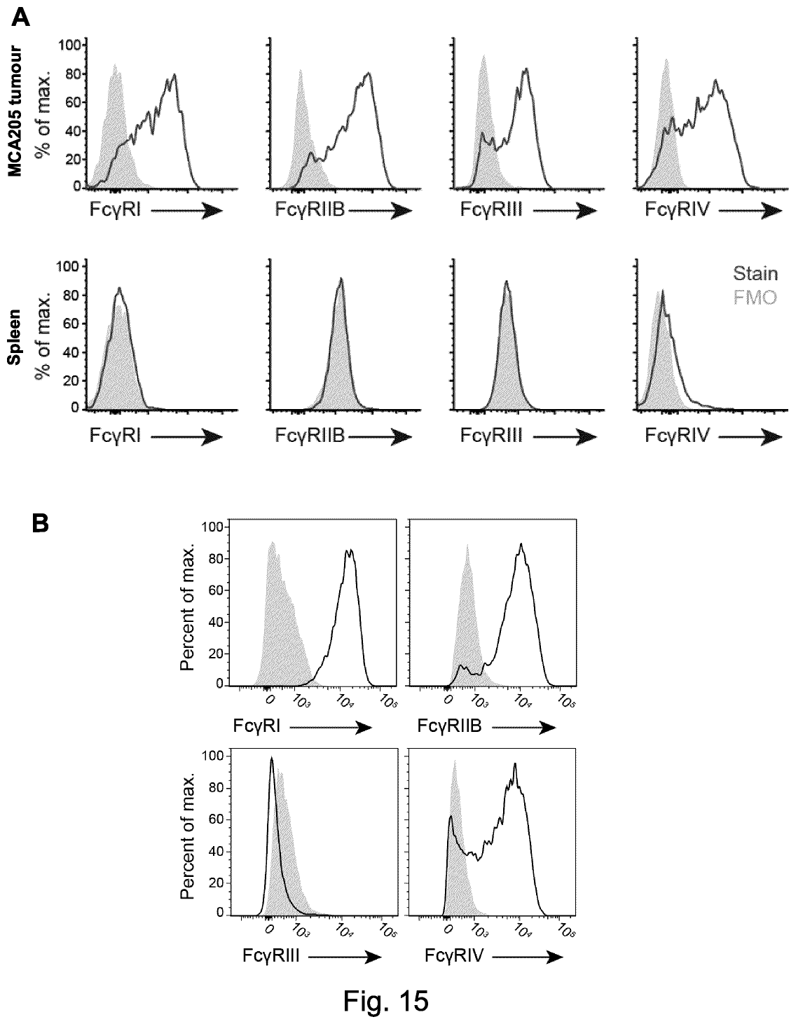

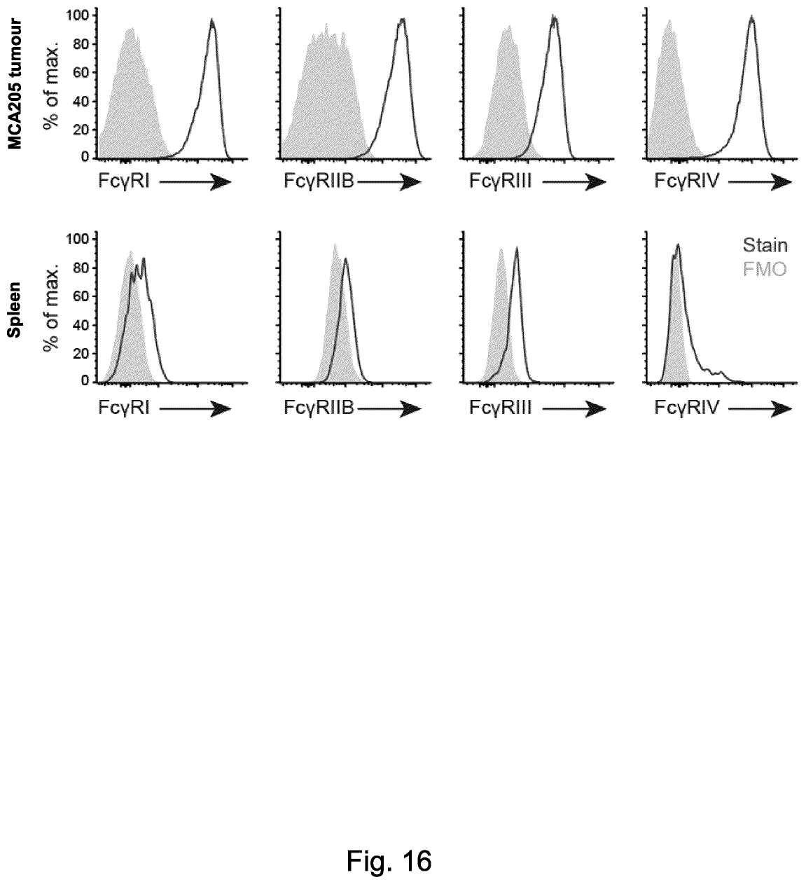

To further highlight the applicability of the mC9-Fc fusion protein in a tumour setting, the expression of different cDC subsets and Fc receptors in tumours was investigated. FIG. 13 shows that analysis of intra-tumoural DC frequency demonstrates that cDC1 represent a minor population of antigen presenting cells, compared to cDC2 and monocyte-derived cells, within tumours in both humans (FIG. 13A) and mice (FIG. 13B). While intra-tumoural XCR1⁺ cDC1 express low levels of FcγR (FIG. 14), CD172a⁺ cDC2 (FIG. 15A) and CD88⁺ MC (FIG. 16) express high levels of all activating FcγR (RI, RIII, RIV), as well as inhibitory FcγRIIB. This expression pattern is largely mirrored in in vitro models, such as HoxB8-derived cDC2 (FIG. 15B).

### Example 13—mC9-Fc Improves Phagocytosis in cDC1 and cDC2

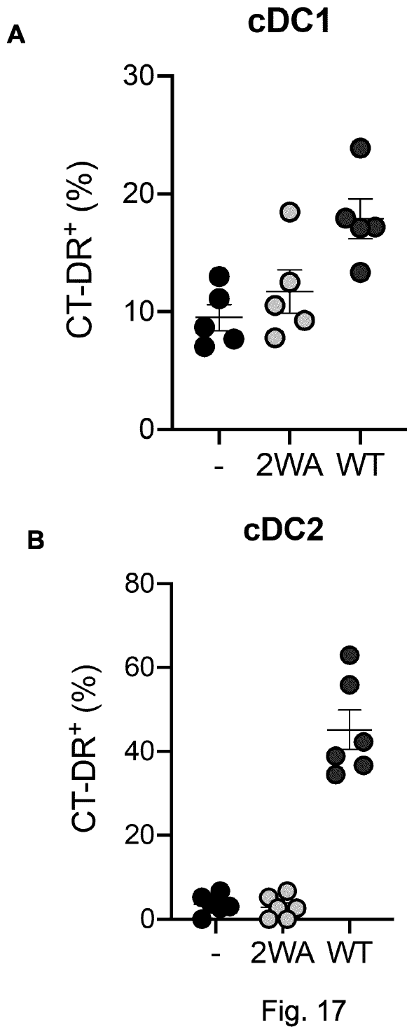

Following on from Example 2 above, which showed mC9-Fc improved phagocytosis in cDC1 and cDC2, the phagocytosis assay was repeated and, in contrast to Example 2 which assessed phagocytosis by flow cytometry, the results in this experiment were assessed using confocal microscopy. FIG. 17 shows that mC9-Fc boosts phagocytosis of dead cell material by both cDC1 (FIG. 17A) and cDC2 (FIG. 17B). This finding is important because cDC2 cells, which are not known to cross present dead cell associated antigens, are abundant in tumours (FIG. 13) and therefore highlights the applicability of such bispecific agents in a tumour setting.

### Example 14—mC9-Fc Promotes Sensing of Dead Cell-Associated Nucleic Acids

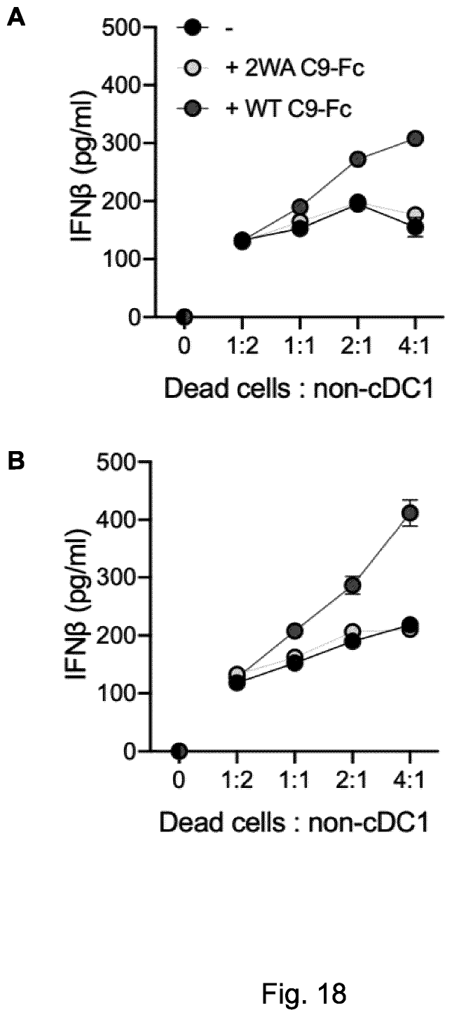

To investigate the mechanism by which mC9-Fc promotes sensing of dead cell-associated nucleic acids, IFNβ production by FLT3L-non-cDC1 (cDC2) treated with poly(I:C)-soaked necrotic cells in the presence of mC9-Fc fusion proteins was measured. While WT mC9-Fc enhances sensing of necrotic cell-associated nucleic acids by non-cDC1, as assessed by IFNβ production, this is independent of cytosolic adaptor protein MAVS (FIG. 18B), which acts downstream of RIG-I and MDA5. Therefore, mC9-Fc does not act via a MAVS-dependent pathway.

### Example 15—mC9-Fc Reduces Tumour Growth In Vivo

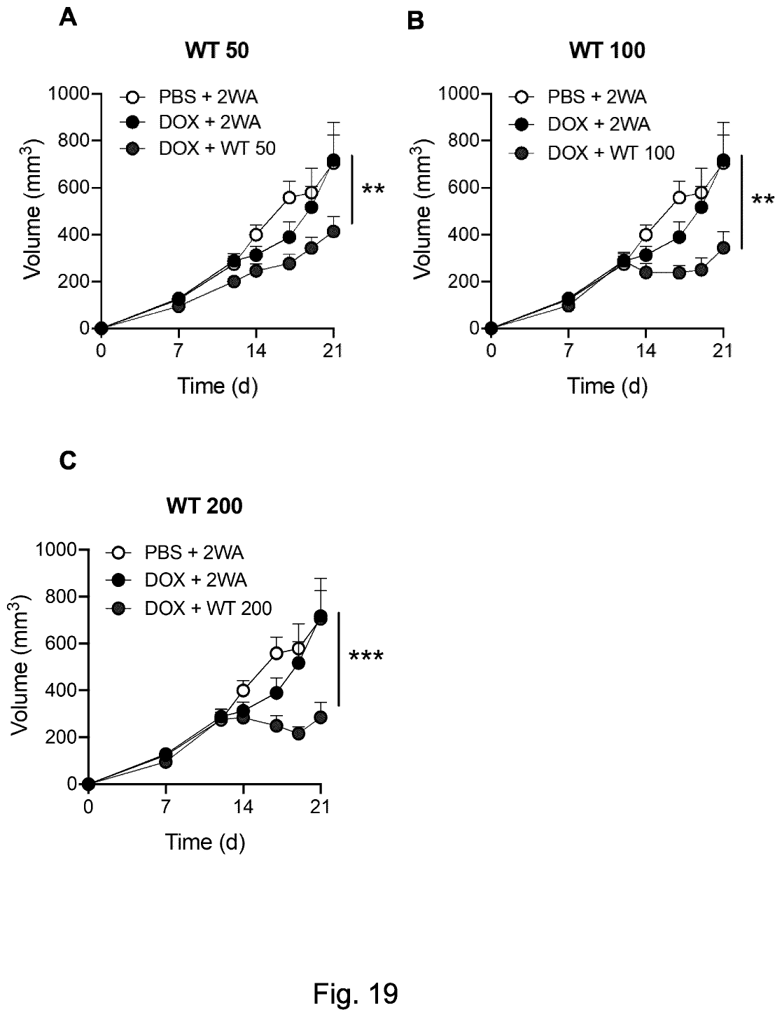

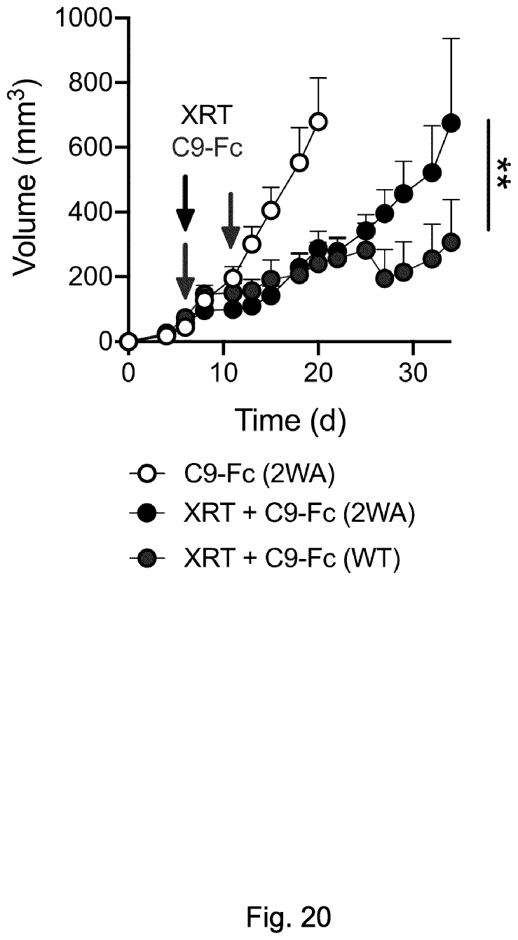

Following on from Example 9 above, the in vivo experiment was repeated using 3 different doses of doxorubicin. FIG. 19 shows that WT mC9-Fc delayed MCA205 tumour growth when administered with doxorubicin intratumourally, at all 3 doses tested. This effect was also seen when WT mC9-Fc was administered in combination with localised X-ray radiotherapy (FIG. 20).

FIGS. 19 and 20 show that WT mC9-Fc attenuates tumour growth in combination with immunogenic cell death-inducing regimens, such as doxorubicin and radiotherapy.

### Example 16—mC9-hFc Fusion Protein can Bind to Dead Cells and Trigger Human FcγR

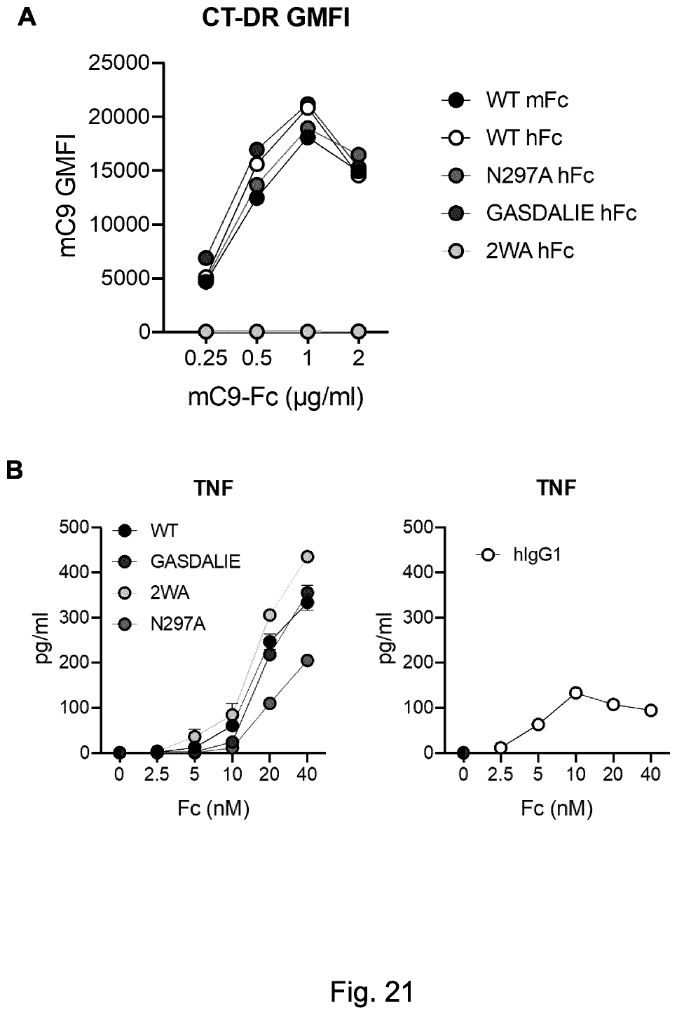

To further investigate why the commercially available hC9-hFc construct was unable to trigger mouse Fcγ receptors on RAW264.7 cells (FIG. 11B), nor augment XP in WT mouse FLT3L cDC cells (FIG. 11C), the inventors constructed murine DNGR-1-human Fc constructs (mC9-hFc). WT, 2WA and N297A mutants were prepared. The in-house mC9-hFc reagents bind comparably to UV-irradiated 5555 necrotic cells (FIG. 21A) and trigger hFcγR (FIG. 21B), except 2WA and N297A mC9-hFc, which show abolished binding to dead cells and attenuated hFcγR triggering, respectively, as expected.

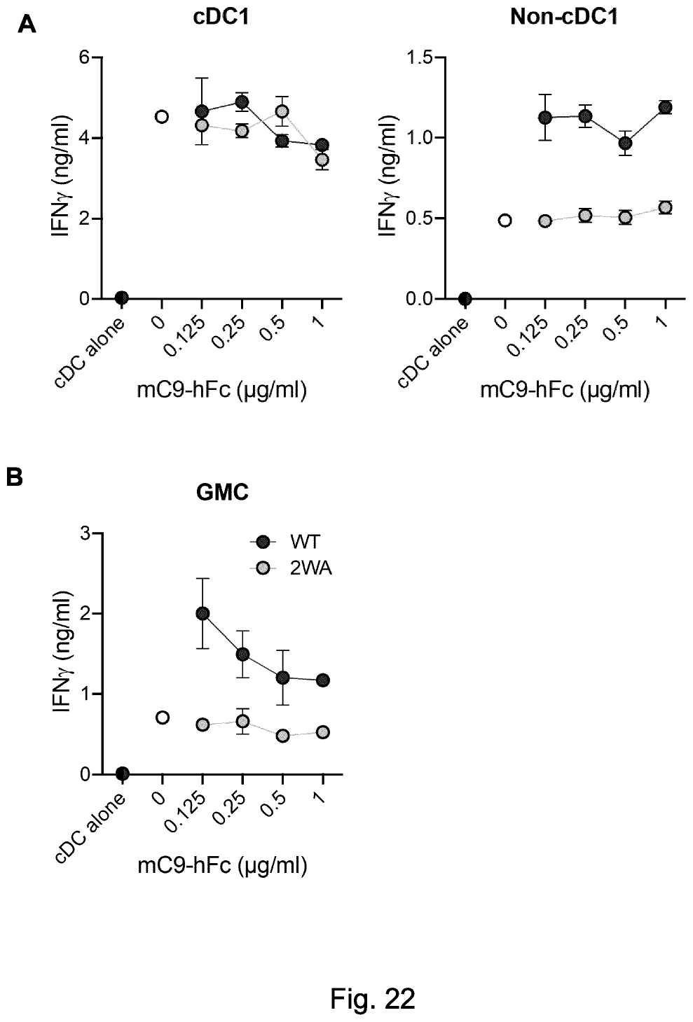

WT mC9-hFc boosts XP by hFcγR-expressing non-cDC1 (which is largely due to cDC2) and GMC, compared to 2WA mC9-hFc (FIG. 22).

The findings of FIGS. 21 and 22 support the hypothesis presented in Example 10 above.

### Example 17—Anti-Actin IgG2a Promotes Dead Cell Cross-Presentation

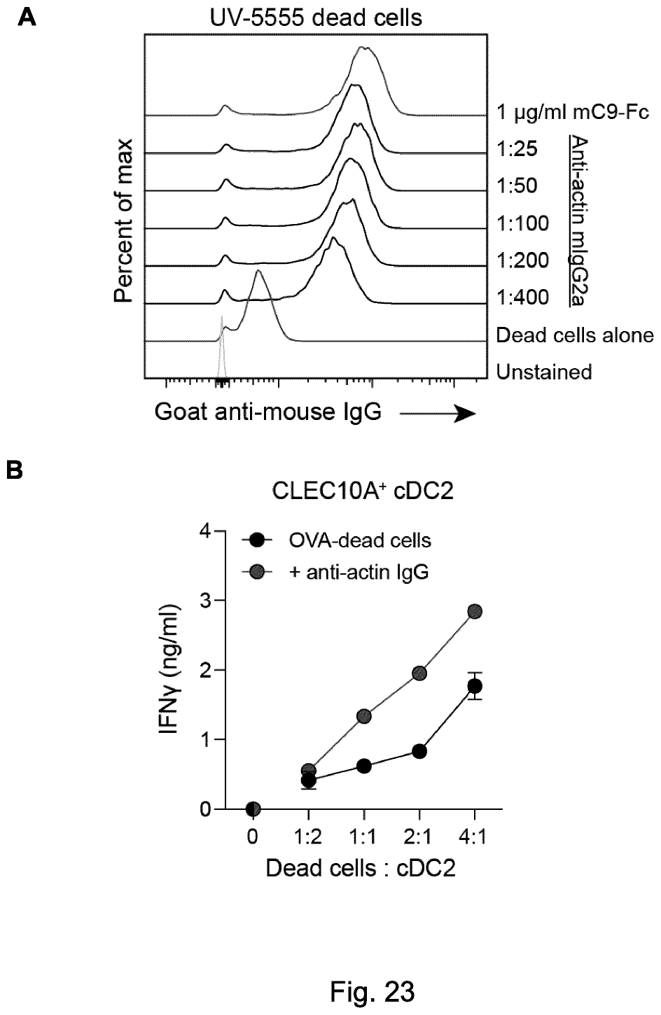

To establish that actin-targeting agents besides the CTLD of DNGR-1 are able to bind dead cells and enhance XP, a commercially available antibody mouse anti-actin IgG2a (AC-40, Abcam) was incubated with UV-irradiated 5555 dead cells and binding was detected. FIG. 23A shows that the antibody is able to bind to the dead cells. FIG. 23B shows that the antibody enhances XP by primary murine FLT3L-derived cDC2.

This data shows that actin-targeting agents other than DNGR-1-Fc are able to enhance XP in cDC2.

### NUMBERED CLAUSES

1. A bispecific binding agent comprising an F-actin binding moiety and an Fc receptor-binding moiety, for use in medicine, wherein the bispecific binding agent is capable of simultaneously binding F-actin via the F-actin binding moiety and binding an Fc receptor via the Fc receptor-binding moiety.
2. The bispecific binding agent for use according to clause 1, wherein the Fc receptor-binding moiety preferentially binds an activatory Fc receptor relative to an inhibitory Fc receptor.
3. The bispecific binding agent for use according to clause 1 or 2, wherein the F-actin binding moiety comprises the C-type lectin-like domain (CTLD) of DNGR-1.
4. The bispecific binding agent for use according to clause 3, wherein the CTLD of DNGR-1 is the CTLD of human DNGR-1.
5. The bispecific binding agent for use according to any preceding clause, wherein the F-actin binding moiety is linked to the Fc receptor-binding moiety via a linker that allows the Fc receptor-binding moiety to bind an Fc receptor and allows the F-actin binding moiety to bind F-actin at the same time.
6. The bispecific binding agent for use according to any preceding clause, wherein the Fc receptor-binding moiety comprises an Fc domain.
7. The bispecific binding agent for use according to clause 6, wherein the Fc domain comprises an Fc domain of an immunoglobulin that is capable of binding and triggering activatory Fc receptors.
8. The bispecific binding agent for use according to clause 6 or 7, wherein the Fc domain is a human Fc domain.
9. The bispecific binding agent for use according to clause 8, wherein the Fc domain is the Fc domain of the human pro-inflammatory immunoglobulin IgG1 or human IgG3.
10. The bispecific binding agent for use according to any one of clauses 5 to 7, wherein the Fc domain is the Fc domain of the murine pro-inflammatory immunoglobulin IgG2a.
11. The bispecific binding agent for use according to any one of clauses 5 to 10, wherein the Fc domain comprises an amino acid sequence that has been mutated from that of a wild type Fc domain such that an Fc receptor binds to the mutated Fc domain with higher affinity and/or avidity than the affinity and/or avidity of the Fc receptor for the wild type Fc domain.
12. The bispecific binding agent for use according to any one of clauses 5 to 9, wherein the mutant Fc domain is a mutant human IgG1 GASDALIE Fc domain, comprising mutations corresponding to G236A, S239D, A330L and I332E.
13. The bispecific binding agent for use according to any one of clauses 5 to 12, wherein the Fc domain binds to an Fcγ receptor.
14. The bispecific binding agent for use according to clause 13, wherein the Fc domain binds to a human Fcγ receptor.
15. The bispecific binding agent for use according to any one of clauses 5 to 14, wherein the linker is a peptide linker.
16. The bispecific binding agent for use according to clause 15, wherein the linker comprises the amino acid sequence GGGGSGGGGS.
17. The bispecific binding agent for use according to clause 16, wherein the linker comprises ARTGGGGSGGGGSDI.
18. The bispecific binding agent for use according to clause 15, wherein the linker is not IEGR.
19. The bispecific binding agent for use according to any one of clauses 15 to 18, wherein the linker does not comprise a helical domain that extends unbroken across more than 60% of the length of the peptide linker.
20. The bispecific binding agent for use according to any one of clauses 15 to 19, wherein the linker comprises at least 10, at least 12, at least 14, at least 16, at least 18 or at least 20 amino acids that do not form part of a helical domain.
21. The bispecific binding agent for use according to any one of clauses 15 to 20, wherein the linker does not comprise the neck region of human DNGR-1.
22. The bispecific binding agent for use according to any one of clauses 15 to 21, wherein the linker comprises the neck region of a mouse DNGR-1.
23. The bispecific binding agent for use according to any one of clauses 5 to 14, wherein the linker is a non-peptide linker.
24. The bispecific binding agent for use according to any preceding clause, wherein the bispecific binding agent is an antibody that binds actin and binds an Fc receptor, wherein the antibody does not block interaction of F-actin with DNGR-1.
25. The bispecific binding agent according to clause 24, wherein the antibody only binds F-actin and does not bind G-actin.
26. The bispecific binding agent for use according to any preceding clause, wherein the use in medicine comprises cancer therapy.
27. The bispecific binding agent for use according to clause 26, wherein the bispecific binding agent is administered in combination with an immunogenic cancer treatment.
28. The bispecific binding agent for use according to clause 27, wherein the immunogenic cancer treatment is radiotherapy or immunogenic chemotherapy.
29. The bispecific binding agent for use according to clause 27 or clause 28, wherein the immunogenic cancer treatment comprises a checkpoint inhibitor.
30. The bispecific binding agent for use according to any one of clauses 26 to 29, wherein the bispecific binding agent is administered to the patient intratumorally (IT), intramuscularly (IM) or intravenously (IV).
31. The bispecific binding agent for use according to any one of clauses 26 to 30, wherein the cancer is characterised by F-actin presentation on necrotic cancer cells.
32. The bispecific binding agent for use according to any preceding clause, wherein the use in medicine comprises treating a viral infection characterised by F-actin presentation on necrotic cells.
33. The bispecific binding agent for use according to clause 32, wherein the antigen is a viral antigen.
34. The bispecific binding agent for use according to clause 1, wherein
   i) the F-actin binding moiety is selected from the group consisting of an aptamer, an affimer, a Fab fragment, a single chain Fc fragment (ScFv), a single domain antibody (sdAb), a variable domain (Fv) and the C-type lectin-like domain (CTLD) of DNGR-1; and
   ii) the Fc receptor-binding moiety is selected from the group consisting of an aptamer, an affimer, a Fab fragment, a single chain Fc fragment (ScFv), a single domain antibody (sdAb), a variable domain (Fv) and an Fc domain.
35. A bispecific binding agent comprising an F-actin binding moiety and an Fc receptor-binding moiety, wherein the bispecific binding agent is capable of simultaneously binding F-actin via the F-actin binding moiety and binding an Fc receptor via the Fc receptor-binding moiety, and wherein:
a) the F-actin binding moiety is the CTLD of human DNGR-1; or
b) the F-actin binding moiety is an affimer specific for F-actin;
and/or
c) the Fc receptor-binding moiety preferentially binds an activatory Fc receptor relative to an inhibitory Fc receptor and the F-actin binding moiety has greater specificity for F-actin relative to G-actin.
36. The bispecific binding agent according to clause 35, wherein the F-actin binding moiety is linked to the Fc receptor-binding moiety via a linker that allows the Fc receptor-binding moiety to bind an Fc receptor and allows the F-actin binding moiety to bind F-actin at the same time.
37. The bispecific binding agent according to clause 35 or clause 36, wherein the Fc receptor-binding moiety comprises an Fc domain.
38. The bispecific binding agent according to clause 37, wherein the Fc domain is a human Fc domain.
39. The bispecific binding agent according to clause 38, wherein the Fc domain is the Fc domain of the human pro-inflammatory immunoglobulin IgG1 or human IgG3.
40. The bispecific binding agent according to clause 36 or 37, wherein the Fc domain is the Fc domain of the murine pro-inflammatory immunoglobulin IgG2a.
41. The bispecific binding agent according to any one of clauses 37 to 40, wherein the Fc domain comprises an amino acid sequence that has been mutated from that of a wild type Fc domain such that an Fc receptor binds to the mutated Fc domain with higher affinity and/or avidity than the affinity and/or avidity of the Fc receptor for the wild type Fc domain.
42. The bispecific binding agent according to clause 41, wherein the mutant Fc domain is a mutant human IgG1 GASDALIE Fc domain, comprising mutations corresponding to G236A, S239D, A330L and I332E.
43. The bispecific binding agent according to any one of clauses 35 to 42, wherein the Fc receptor-binding moiety binds to an Fcγ receptor.
44. The bispecific binding agent according to clause 43, wherein the Fc receptor-binding moiety binds to a human Fcγ receptor.
45. The bispecific binding agent according to any one of clauses 36 to 44, wherein the linker is a peptide linker.
46. The bispecific binding agent according to clause 45, wherein the linker comprises the amino acid sequence GGGGSGGGGS.
47. The bispecific binding agent according to clause 46, wherein the linker comprises ARTGGGGSGGGGSDI.
48. The bispecific binding agent according to any one of clauses 45 to 47, wherein the linker is not IEGR.
49. The bispecific binding agent according to any one of clauses 45 to 48, wherein the linker does not comprise a helical domain that extends unbroken across more than 60% of the length of the peptide linker.
50. The bispecific binding agent according to any one of clauses 45 to 49, wherein the linker comprises at least 10, at least 12, at least 14, at least 16, at least 18 or at least 20 amino acids that do not form part of a helical domain.
51. The bispecific binding agent according to any one of clauses 45 to 50, wherein the linker does not comprise the neck region of human DNGR-1.
52. The bispecific binding agent according to any one of clauses 45 to 51, wherein the linker comprises the neck region of a mouse DNGR-1.
53. The bispecific binding agent according to any one of clauses 36 to 44, wherein the linker is a non-peptide linker.
54. The bispecific binding agent according to any one of clauses 35 to 53, wherein the bispecific binding agent is an antibody that binds actin and binds an Fc receptor, wherein the antibody does not block interaction of F-actin with DNGR-1.
55. The bispecific binding agent according to clause 54, wherein the antibody only binds F-actin and does not bind G-actin.
56. The bispecific binding agent according to clause 35, wherein the Fc receptor-binding moiety is selected from the group consisting of an aptamer, an affimer, a Fab fragment, a single chain Fc fragment (ScFv), a single domain antibody (sdAb), a variable domain (Fv) and an Fc domain.
57. A method for identifying a target tumour antigen, the method comprising contacting the bispecific binding agent according to any one of clauses 35 to 56 with a tumour biopsy sample, and analysing peptide epitopes that are cross presented on MHC class I molecules on antigen presenting cells present in the sample.
58. The method according to clause 57, comprising identifying the immunodominant epitope of the target tumour antigen.
59. A nucleic acid encoding the bispecific binding agent according to any one of clauses 35 to 56.
60. A cell comprising the nucleic acid according to clause 59.
61. A plasmid for making the bispecific binding agent of any one of clauses 35 to 56.

### REFERENCES

A number of publications are cited above in order to more fully describe and disclose the invention and the state of the art to which the invention pertains. Full citations for these references are provided below. The entirety of each of these references is incorporated herein.
Ahrens, S. et al. F-actin is an evolutionarily conserved damage-associated molecular pattern recognized by DNGR-1, a receptor for dead cells. Immunity 36, 635-645 (2012)
Alloatti, A., et al., Critical role for Sec22b-dependent antigen crosspresentation in antitumor immunity. Journal of Experimental Medicine, 2017. 214 (8): p. 2231-2241.
Bruhns, P and Jonsson, F. Mouse and human FcR effector functions. Immunological Reviews, 268:25-51 (2015)
Canton, J., et al., The receptor DNGR-1 signals for phagosomal rupture to promote cross-presentation of dead-cell-associated antigens. Nat Immunol, 2021. 22 (2): p. 140-153.
Carmi, Y., et al., Allogeneic IgG combined with dendritic cell stimuli induce antitumour T-cell immunity. Nature, 2015: p. 1-6.
Carmi, Y. et al. Tumor-binding antibodies induce potent dendritic cell-mediated tumor immunity. Oncoimmunology 8 (10), e1078063 (2019)
Castro-Dopico, T. & Clatworthy, M. IgG and Fcγ Receptors in Intestinal Immunity and Inflammation. Frontiers in Immunology 10:805 (2019)
Cheng et al. A pan-cancer single-cell transcriptional atlas of tumour infiltrating myeloid cells. Cell, 2021. 184 (3): p. 792-809.
Galluzzi, L., et al., Immunogenic cell death in cancer and infectious disease. Nature Reviews Immunology, 2017. 17 (2): p. 97-111.
Freund, E. C., et al., Efficient gene knockout in primary human and murine myeloid cells by non-viral delivery of crispr-cas9. Journal of Experimental Medicine, 2020. 217 (7).
Giampazolias, E., et al., Secreted gelsolin inhibits DNGR-1-dependent cross-presentation and cancer immunity. Cell, 2021. 184 (15): p. 4016-4031.e22.
Guilliams, M., et al., The function of Fcγ receptors in dendritic cells and macrophages. Nature reviews. Immunology, 2014. 14 (2): p. 94-108.
Hanč, P., et al., Structure of the Complex of F-Actin and DNGR-1, a C-Type Lectin Receptor Involved in Dendritic Cell Cross-Presentation of Dead Cell-Associated Antigens. Immunity, 2015. 42 (5): p. 839-849. Helft, J., et al., GM-CSF Mouse Bone Marrow Cultures Comprise a Heterogeneous Population of CD11c+ MHCII+ Macrophages and Dendritic Cells. Immunity, 2015. 42 (6): p. 1197-1211.
Huysamen, C., et al., CLEC9A is a novel activation C-type lectin-like receptor expressed on BDCA3+ dendritic cells and a subset of monocytes. Journal of Biological Chemistry, 2008. 283 (24): p. 16693-16701.
Iborra, S., et al., The DC receptor DNGR-1 mediates cross-priming of CTLs during vaccinia virus infection in mice. Journal of Clinical Investigation, 2012. 122 (5): p. 1628-1643.
Kirkling, M. E., et al., Notch Signaling Facilitates In Vitro Generation of Cross-Presenting Classical Dendritic Cells. Cell Reports, 2018. 23 (12): p. 3658-3672.e6.
Mayer, S., et al., C-Type Lectin Receptor (CLR)-Fc Fusion Proteins As Tools to Screen for Novel CLR/Bacteria Interactions: An Exemplary Study on Preselected Campylobacter jejuni Isolates. Front. Immunol. (2018) 9:213.doi: 10.3389/fimmu.2018.00213
Poulin, L. F., et al., DNGR-1 is a specific and universal marker of mouse and human Batf3-dependent dendritic cells in lymphoid and nonlymphoid tissues. Blood, 2012. 119 (25): p. 6052-6062.
Regnault, A., et al., Fcgamma receptor-mediated induction of dendritic cell maturation and major histocompatibility complex class I-restricted antigen presentation after immune complex internalization. The Journal of experimental medicine, 1999. 189 (2): p. 371-80.
Salmon, H., et al., Expansion and Activation of CD103+ Dendritic Cell Progenitors at the Tumor Site Enhances Tumor Responses to Therapeutic PD-L1 and BRAF Inhibition. Immunity, 2016. 44 (4): p. 924-938.
Sancho, D., et al., Tumor therapy in mice via antigen targeting to a novel, DC-restricted C-type lectin. Journal of Clinical Investigation, 2008. 118 (6): p. 2098-2110.
Sancho, D. et al. Identification of a dendritic cell receptor that couples sensing of necrosis to immunity. Nature 458, 899-903 (2009)
Saunders, K. O. Conceptual Approaches to Modulating Antibody Effector Functions and Circulation Half-Life. Front. Immunol. (2019) 10:1296. doi: 10.3389/fimmu.2019.01296
Theisen, D. J., et al., WDFY4 is required for cross-presentation in response to viral and tumor antigens. Science, 2018. 362 (6415): p. 694-699
Uto, T., et al., Clec4A4 is a regulatory receptor for dendritic cells that impairs inflammation and T-cell immunity. Nature Communications, 2016. 7
Wculek, S. K., et al., Dendritic cells in cancer immunology and immunotherapy. Nature Reviews Immunology, 2020. 20 (1): p. 7-24.
WO 2009/013484A1
WO 2013/088136
WO 2022/163809
Zhang, J.-G. et al. The dendritic cell receptor Clec9A binds damaged cells via exposed actin filaments. Immunity 36, 646-657 (2012).

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A bispecific binding agent comprising an F-actin binding moiety and an Fc domain for use in medicine,
wherein the Fc domain comprises an amino acid sequence that has been mutated from that of a wild type Fc domain such that the mutated Fc domain binds to an Fc receptor with higher affinity and/or avidity than the affinity and/or avidity of the wild type Fc domain for the Fc receptor;
wherein the bispecific binding agent is capable of simultaneously binding F-actin via the F-actin binding moiety and binding an Fc receptor via the Fc domain.

2. The bispecific binding agent for use according to claim 1, wherein the Fc receptor is an activatory Fc receptor.

3. The bispecific binding agent for use according to claim 1 or 2, wherein the Fc domain preferentially binds an activatory Fc receptor relative to an inhibitory Fc receptor.

4. The bispecific binding agent for use according to any preceding claim, wherein the F-actin binding moiety comprises the C-type lectin-like domain (CTLD) of DNGR-1.

5. The bispecific binding agent for use according to claim 4, wherein the CTLD of DNGR-1 is the CTLD of human DNGR-1.

6. The bispecific binding agent for use according to any preceding claim, wherein the F-actin binding moiety is linked to the Fc domain via a linker that allows the Fc domain to bind an Fc receptor and allows the F-actin binding moiety to bind F-actin at the same time.

7. The bispecific binding agent for use according to any preceding claim, wherein the Fc domain comprises an Fc domain of an immunoglobulin that is capable of binding and triggering activatory Fc receptors.

8. The bispecific binding agent for use according to any preceding claim, wherein the Fc domain is a human Fc domain.

9. The bispecific binding agent for use according to claim 8, wherein the Fc domain is the Fc domain of the human pro-inflammatory immunoglobulin IgG1 or human IgG3.

10. The bispecific binding agent for use according to any one of claims 1 to 7, wherein the Fc domain is the Fc domain of the murine pro-inflammatory immunoglobulin IgG2a.

11. The bispecific binding agent for use according to any one of claims 1 to 9, wherein the mutant Fc domain is a mutant human IgG1 GASDALIE Fc domain, comprising mutations corresponding to G236A, S239D, A330L and I332E.

12. The bispecific binding agent for use according to any preceding claim, wherein the Fc domain binds to an Fcγ receptor.

13. The bispecific binding agent for use according to claim 12, wherein the Fc domain binds to a human Fcγ receptor.

14. The bispecific binding agent for use according to any one of claims 6 to 13, wherein the linker is a peptide linker.

15. The bispecific binding agent for use according to claim 14, wherein the linker comprises the amino acid sequence GGGGSGGGGS.

16. The bispecific binding agent for use according to claim 14, wherein the linker comprises ARTGGGGSGGGGSDI.

17. The bispecific binding agent for use according to claim 14, wherein the linker comprises the amino acid sequence SGAGSNTSTSTGTSTSSSGPSSG.

18. The bispecific binding agent for use according to claim 14, wherein the linker comprises the amino acid sequence AEAAARAEAAARAEAAARAPPS.

19. The bispecific binding agent for use according to claim 14, wherein the linker comprises the amino acid sequence AEAAARAEAAARAEAAARAEAAARAAEAAARAEAAARAEAAARAEAAARAPPS.

20. The bispecific binding agent for use according to claim 14, wherein the linker is not IEGR.

21. The bispecific binding agent for use according to any one of claims 14 to 20, wherein the linker does not comprise a helical domain that extends unbroken across more than 60% of the length of the peptide linker.

22. The bispecific binding agent for use according to any one of claims 14 to 21, wherein the linker comprises at least 10, at least 12, at least 14, at least 16, at least 18 or at least 20 amino acids that do not form part of a helical domain.

23. The bispecific binding agent for use according to any one of claims 14 to 22, wherein the linker does not comprise the neck region of human DNGR-1.

24. The bispecific binding agent for use according to any one of claims 14 to 23, wherein the linker comprises the neck region of a mouse DNGR-1.

25. The bispecific binding agent for use according to any one of claims 6 to 13, wherein the linker is a non-peptide linker.

26. The bispecific binding agent for use according to any preceding claim, wherein the bispecific binding agent is an antibody that binds actin and binds an Fc receptor, wherein the antibody does not block interaction of F-actin with DNGR-1.

27. The bispecific binding agent according to claim 26, wherein the antibody only binds F-actin and does not bind G-actin.

28. The bispecific binding agent for use according to any preceding claim, wherein the use in medicine comprises cancer therapy.

29. The bispecific binding agent for use according to claim 28, wherein the bispecific binding agent is administered in combination with an immunogenic cancer treatment.

30. The bispecific binding agent for use according to claim 29, wherein the immunogenic cancer treatment is radiotherapy or immunogenic chemotherapy.

31. The bispecific binding agent for use according to claim 29 or claim 30, wherein the immunogenic cancer treatment comprises a checkpoint inhibitor.

32. The bispecific binding agent for use according to any one of claims 28 to 31, wherein the bispecific binding agent is administered to the patient intratumorally (IT), intramuscularly (IM) or intravenously (IV).

33. The bispecific binding agent for use according to any one of claims 28 to 32, wherein the cancer is characterised by F-actin presentation on necrotic cancer cells.

34. The bispecific binding agent for use according to any preceding claim, wherein the use in medicine comprises treating a viral infection characterised by F-actin presentation on necrotic cells.

35. The bispecific binding agent for use according to claim 1, wherein the F-actin binding moiety is selected from the group consisting of an aptamer, an affimer, a Fab fragment, a single chain Fc fragment (ScFv), a single domain antibody (sdAb), a variable domain (Fv) and the C-type lectin-like domain (CTLD) of DNGR-1.

36. A bispecific binding agent comprising an F-actin binding moiety and an Fc domain that comprises an amino acid sequence that has been mutated from that of a wild type Fc domain such that the mutated Fc domain binds to an Fc receptor with higher affinity and/or avidity than the affinity and/or avidity of the wild type Fc domain for the Fc receptor, wherein the bispecific binding agent is capable of simultaneously binding F-actin via the F-actin binding moiety and binding an Fc receptor via the Fc domain, and wherein:
a) the F-actin binding moiety is the CTLD of human DNGR-1; or
b) the F-actin binding moiety is an affimer specific for F-actin;
and/or
c) the Fc domain preferentially binds an activatory Fc receptor relative to an inhibitory Fc receptor and the F-actin binding moiety has greater specificity for F-actin relative to G-actin.

37. The bispecific binding agent according to claim 36, wherein the Fc receptor that the mutated Fc domain binds to with higher affinity and/or avidity than the affinity and/or avidity of the wild type Fc domain for the Fc receptor is an activatory Fc receptor.

38. The bispecific binding agent according to claim 36 or 37, wherein the F-actin binding moiety is linked to the Fc domain via a linker that allows the Fc domain to bind an Fc receptor and allows the F-actin binding moiety to bind F-actin at the same time.

39. The bispecific binding agent according to any one of claims 36 to 38, wherein the Fc domain is a human Fc domain.

40. The bispecific binding agent according to claim 39, wherein the Fc domain is the Fc domain of the human pro-inflammatory immunoglobulin IgG1 or human IgG3.

41. The bispecific binding agent according to any one of claims 36 to 38, wherein the Fc domain is the Fc domain of the murine pro-inflammatory immunoglobulin IgG2a.

42. The bispecific binding agent according to any one of claims 39 to 41, wherein the mutant Fc domain is a mutant human IgG1 GASDALIE Fc domain, comprising mutations corresponding to G236A, S239D, A330L and I332E.

43. The bispecific binding agent according to any one of claims 36 to 42, wherein the Fc domain binds to an Fcγ receptor.

44. The bispecific binding agent according to claim 43, wherein the Fc domain binds to a human Fcγ receptor.

45. The bispecific binding agent according to any one of claims 38 to 44, wherein the linker is a peptide linker.

46. The bispecific binding agent according to claim 45, wherein the linker comprises the amino acid sequence GGGGSGGGGS.

47. The bispecific binding agent according to claim 46, wherein the linker comprises ARTGGGGSGGGGSDI.

48. The bispecific binding agent according to claim 45, wherein the linker comprises the amino acid sequence SGAGSNTSTSTGTSTSSSGPSSG.

49. The bispecific binding agent according to claim 45, wherein the linker comprises the amino acid sequence AEAAARAEAAARAEAAARAPPS.

50. The bispecific binding agent according to claim 45, wherein the linker comprises the amino acid sequence AEAAARAEAAARAEAAARAEAAARAAEAAARAEAAARAEAAARAEAAARAPPS.

51. The bispecific binding agent according to any one of claims 45 to 50, wherein the linker is not IEGR.

52. The bispecific binding agent according to any one of claims 45 to 51, wherein the linker does not comprise a helical domain that extends unbroken across more than 60% of the length of the peptide linker.

53. The bispecific binding agent according to any one of claims 45 to 52, wherein the linker comprises at least 10, at least 12, at least 14, at least 16, at least 18 or at least 20 amino acids that do not form part of a helical domain.

54. The bispecific binding agent according to any one of claims 45 to 53, wherein the linker does not comprise the neck region of human DNGR-1.

55. The bispecific binding agent according to any one of claims 45 to 54, wherein the linker comprises the neck region of a mouse DNGR-1.

56. The bispecific binding agent according to any one of claims 38 to 44, wherein the linker is a non-peptide linker.

57. The bispecific binding agent according to any one of claims 36 to 56, wherein the bispecific binding agent is an antibody that binds actin and binds an Fc receptor, wherein the antibody does not block interaction of F-actin with DNGR-1.

58. The bispecific binding agent according to claim 57, wherein the antibody only binds F-actin and does not bind G-actin.

59. A method for identifying a target tumour antigen, the method comprising contacting the bispecific binding agent according to any one of claims 36 to 58 with a tumour biopsy sample, and analysing peptide epitopes that are cross presented on MHC class I molecules on antigen presenting cells present in the sample.

60. The method according to claim 59, comprising identifying the immunodominant epitope of the target tumour antigen.

61. A nucleic acid encoding the bispecific binding agent according to any one of claims 36 to 58.

62. A cell comprising the nucleic acid according to claim 61.

63. A plasmid for making the bispecific binding agent of any one of claims 36 to 58.

64. A bispecific binding agent comprising an F-actin binding moiety and an Fc receptor-binding moiety, for use in medicine, wherein the bispecific binding agent is capable of simultaneously binding F-actin via the F-actin binding moiety and binding an Fc receptor via the Fc receptor-binding moiety, wherein
i) the F-actin binding moiety is selected from the group consisting of an aptamer, an affimer, a Fab fragment, a single chain Fc fragment (ScFv), a single domain antibody (sdAb), a variable domain (Fv) and the C-type lectin-like domain (CTLD) of DNGR-1; and
ii) the Fc receptor-binding moiety is selected from the group consisting of an aptamer, an affimer, a Fab fragment, a single chain Fc fragment (ScFv), a single domain antibody (sdAb) and a variable domain (Fv).

65. A bispecific binding agent comprising an F-actin binding moiety and an Fc receptor-binding moiety, wherein the bispecific binding agent is capable of simultaneously binding F-actin via the F-actin binding moiety and binding an Fc receptor via the Fc receptor-binding moiety, and wherein:
a) the F-actin binding moiety is the CTLD of human DNGR-1; or
b) the F-actin binding moiety is an affimer specific for F-actin;
and/or
c) the Fc receptor-binding moiety preferentially binds an activatory Fc receptor relative to an inhibitory Fc receptor and the F-actin binding moiety has greater specificity for F-actin relative to G-actin;
and wherein the Fc receptor-binding moiety is selected from the group consisting of an aptamer, an affimer, a Fab fragment, a single chain Fc fragment (ScFv), a single domain antibody (sdAb) and a variable domain (Fv).

66. The bispecific binding agent for use according to claim 64, or the bispecific binding agent according to claim 65, wherein the Fc receptor-binding moiety preferentially binds an activatory Fc receptor relative to an inhibitory Fc receptor.

67. The bispecific binding agent for use or the bispecific binding agent according to any of claims 64 to 66, wherein the F-actin binding moiety comprises the C-type lectin-like domain (CTLD) of DNGR-1.

68. The bispecific binding agent for use or the bispecific binding agent according to claim 67, wherein the CTLD of DNGR-1 is the CTLD of human DNGR-1.

69. The bispecific binding agent for use or the bispecific binding agent according to any of claims 64 to 68, wherein the F-actin binding moiety is linked to the Fc receptor-binding moiety via a linker that allows the Fc receptor-binding moiety to bind an Fc receptor and allows the F-actin binding moiety to bind F-actin at the same time.

70. The bispecific binding agent for use or the bispecific binding agent according to claim 69, wherein the linker is a peptide linker.

71. The bispecific binding agent for use or the bispecific binding agent according to claim 70, wherein the linker comprises the amino acid sequence GGGGSGGGGS.

72. The bispecific binding agent for use or the bispecific binding agent according to claim 70, wherein the linker comprises ARTGGGGSGGGGSDI.

73. The bispecific binding agent for use or the bispecific binding agent according to claim 70, wherein the linker comprises the amino acid sequence SGAGSNTSTSTGTSTSSSGPSSG.

74. The bispecific binding agent for use or the bispecific binding agent according to claim 70, wherein the linker comprises the amino acid sequence AEAAARAEAAARAEAAARAPPS.

75. The bispecific binding agent for use or the bispecific binding agent according to claim 70, wherein the linker comprises the amino acid sequence AEAAARAEAAARAEAAARAEAAARAAEAAARAEAAARAEAAARAEAAARAPPS.

76. The bispecific binding agent for use or the bispecific binding agent according to claim 70, wherein the linker is not IEGR.

77. The bispecific binding agent for use or the bispecific binding agent according to any one of claims 69 to 76, wherein the linker does not comprise a helical domain that extends unbroken across more than 60% of the length of the peptide linker.

78. The bispecific binding agent for use or the bispecific binding agent according to any one of claims 69 to 77, wherein the linker comprises at least 10, at least 12, at least 14, at least 16, at least 18 or at least 20 amino acids that do not form part of a helical domain.

79. The bispecific binding agent for use or the bispecific binding agent according to any one of claims 69 to 78, wherein the linker does not comprise the neck region of human DNGR-1.

80. The bispecific binding agent for use or the bispecific binding agent according to any one of claims 69 to 79, wherein the linker comprises the neck region of a mouse DNGR-1.

81. The bispecific binding agent for use or the bispecific binding agent according to claim 69, wherein the linker is a non-peptide linker.

82. The bispecific binding agent for use or the bispecific binding agent according to any of claims 64 to 81, wherein the bispecific binding agent is an antibody that binds actin and binds an Fc receptor, wherein the antibody does not block interaction of F-actin with DNGR-1.

83. The bispecific binding agent for use or the bispecific binding agent according to claim 82, wherein the antibody only binds F-actin and does not bind G-actin.

84. The bispecific binding agent for use according to any of claims 64 and 66 to 83, wherein the use in medicine comprises cancer therapy.

85. The bispecific binding agent for use according to claim 84, wherein the bispecific binding agent is administered in combination with an immunogenic cancer treatment.

86. The bispecific binding agent for use according to claim 85, wherein the immunogenic cancer treatment is radiotherapy or immunogenic chemotherapy.

87. The bispecific binding agent for use according to claim 85 or claim 86, wherein the immunogenic cancer treatment comprises a checkpoint inhibitor.

88. The bispecific binding agent for use according to any one of claims 84 to 87, wherein the bispecific binding agent is administered to the patient intratumorally (IT), intramuscularly (IM) or intravenously (IV).

89. The bispecific binding agent for use according to any one of claims 84 to 88, wherein the cancer is characterised by F-actin presentation on necrotic cancer cells.

90. The bispecific binding agent for use according to any one of claims 64 and 66 to 89, wherein the use in medicine comprises treating a viral infection characterised by F-actin presentation on necrotic cells.


SEQUENCE LISTING (25 sequences)

SEQ ID NO: 1 - protein, 238 aa, Mus musculus
MHAEEIYTSLQWDIPTSEASQKCQSPSKCSGAWCVVTMISCVVCMGLLATSIFLGIKFFQVSSLVLEQQERLIQQDTALVNLTQWQRKYTLEYCQALLQRSLHSGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 2 - protein, 53 aa, Mus musculus
KFFQVSSLVLEQQERLIQQDTALVNLTQWQRKYTLEYCQALLQRSLHSGSDCS

SEQ ID NO: 3 - protein, 264 aa, Mus musculus
MHAEEIYTSLQWDIPTSEASQKCQSPSKCSGAWCVVTMISCVVCMGLLATSIFLGIKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 4 - protein, 79 aa, Mus musculus
KFFQVSSLVLEQQERLIQQDTALVNLTQWQRKYTLEYCQALLQRSLHSGTDASTGPVLLTSPQMVPQTLDSKETGSDCS

SEQ ID NO: 5 - protein, 124 aa, Mus musculus
PCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKA

SEQ ID NO: 6 - protein, 241 aa, Homo sapiens
MHEEEIYTSLQWDSPAPDTYQKCLSSNKCSGACCLVMVISCVFCMGLLTASIFLGVKLLQVSTIAMQQQEKLIQQERALLNFTEWKRSCALQMKYCQAFMQNSLSSAHNSSPCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYALRSSV

SEQ ID NO: 7 - protein, 55 aa, Homo sapiens
KLLQVSTIAMQQQEKLIQQERALLNFTEWKRSCALQMKYCQAFMQNSLSSAHNSS

SEQ ID NO: 8 - protein, 125 aa, Homo sapiens
PCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYA

SEQ ID NO: 9 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 226 aa, Homo sapiens
DKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 276 aa, Homo sapiens
TPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 12 - protein, 227 aa, Mus musculus
TIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 13 - protein, 478 aa, synthetic construct
MYRMQLLSCIALSLALVTNSAPLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGARTGGGGSGGGGSDIKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 14 - protein, 478 aa, synthetic construct
MYRMQLLSCIALSLALVTNSAPLEPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGARTGGGGSGGGGSDIKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 15 - protein, 478 aa, synthetic construct
MYRMQLLSCIALSLALVTNSAPLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGARTGGGGSGGGGSDIKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERAEMWNISKKSCLKEGASLFQIDSKEEMEFISSIGKLKGGNKYWVGVFQDGISGSWFWEDGSSPLSDLLPAERQRSAGQICGYLKDSTLISDKCDSAKYFICEKKAFGSCI

SEQ ID NO: 16 - protein, 368 aa, synthetic construct
MYRMQLLSCIALSLALVTNSTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGARTGGGGSGGGGSGGGGSDIASNSLEIEELARFAVDEHNKKENALLEFVRVVKAKEQSSVPHWWWTTMYYLTLEAKDGGKKKLYEAKVWVKRDPNMIFKINFKELQEFKPVGDAAGSTGSR

SEQ ID NO: 17 - protein, 350 aa, synthetic construct
MYRMQLLSCIALSLALVTNSTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGARTGGGGSGGGGSGGGGSDIASNSLEIEELARFAVDEHNKKENALLEFVRVVKAKEQVVAGTMYYLTLEAKDGGKKKLYEAKVWVKPWENFKELQEFKPVGDA

SEQ ID NO: 18 - protein, 368 aa, synthetic construct
MYRMQLLSCIALSLALVTNSTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGARTGGGGSGGGGSGGGGSDIASNSLEIEELARFAVDEHNKKENALLEFVRVVKAKEQSDTPHWWWTTMYYLTLEAKDGGKKKLYEAKVWVKESPVHPKRLNFKDLQEFKPVGDAAGSTGSR

SEQ ID NO: 19 - protein, 368 aa, synthetic construct
MYRMQLLSCIALSLALVTNSTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGARTGGGGSGGGGSGGGGSDIASNSLEIEELARFAVDEHNKKENALLEFVRVVKAKEQMDMIGEYVSTMYYLTLEAKDGGKKKLYEAKVWVKGWMPLYSRQNFKELQEFKPVGDAAGSTGSR

SEQ ID NO: 20 - protein, 10 aa, synthetic construct
GGGGSGGGGS

SEQ ID NO: 21 - protein, 15 aa, synthetic construct
ARTGGGGSGGGGSDI

SEQ ID NO: 22 - protein, 23 aa, synthetic construct
SGAGSNTSTSTGTSTSSSGPSSG

SEQ ID NO: 23 - protein, 22 aa, synthetic construct
AEAAARAEAAARAEAAARAPPS

SEQ ID NO: 24 - protein, 53 aa, synthetic construct
AEAAARAEAAARAEAAARAEAAARAAEAAARAEAAARAEAAARAEAAARAPPS

SEQ ID NO: 25 - protein, 4 aa, synthetic construct
IEGR